(19) United States
(12) Patent Application Publication
(10) Pub. No.: US 2024/0238274 A1
(43) Pub. Date: Jul. 18, 2024

(54) INHIBITORS OF UBIQUITIN SPECIFIC PEPTIDASE 22 (USP22) AND USES THEREOF FOR TREATING DISEASE AND DISORDERS

(71) Applicant: Northwestern University, Evanston, IL (US)

(72) Inventors: Deyu Fang, Evanston, IL (US); Elena Montauti, Evanston, IL (US); Ming Yan, Evanston, IL (US); Beixue Gao, Evanston, IL (US); Amy Tang, Evanston, IL (US); Huiping Liu, Evanston, IL (US)

(21) Appl. No.: 18/556,534

(22) PCT Filed: Apr. 25, 2022
(86) PCT No.: PCT/US22/26159

§ 371 Date: Oct. 20, 2023

Related U.S. Application Data
(60) Provisional application No. 63/201,330, filed on Apr. 23, 2021.

(51) Int. Cl. A61K 31/4745; A61K 31/255; A61K 31/277; A61K 31/343; A61K 31/353; A61K 31/366; A61K 31/37; A61K 31/381; A61K 31/435; A61K 31/4418; A61K 31/4436; A61K 31/4965; A61K 31/517; A61K 31/519; A61P 35/00

(52) U.S. Cl. CPC A61K 31/4745; A61K 31/255; A61K 31/277; A61K 31/343; A61K 31/353; A61K 31/366; A61K 31/37; A61K 31/381; A61K 31/435; A61K 31/4418; A61K 31/4436; A61K 31/4965; A61K 31/517; A61K 31/519; A61P 35/00

(57) ABSTRACT

Disclosed are methods of treating diseases or disorders associated with the expression of Ubiquitin Specific Peptidase 22 (USP22). The disclosed methods may be utilized to treat diseases or disorders associated with cell proliferation, including cancer. Also disclosed are inhibitors of USP22 that specifically inhibit the EC:3.4.19.12 activity, or the thiol-dependent hydrolysis of ester, thioester, amide, peptide and isopeptide bonds formed by the C-terminal glycine of ubiquitin. The disclosed compounds may also be used in pharmaceutical compositions and methods for treatment of cell proliferative diseases or disorders associated with USP22 activity.

### CROSS-REFERENCE TO RELATED APPLICATIONS

This application claims benefit of priority to U.S. Patent Application Ser. No. 63/201,330, filed Apr. 23, 2021, the contents of which are incorporated by reference in its entirety.

### STATEMENT REGARDING FEDERALLY SPONSORED RESEARCH OR DEVELOPMENT

This invention was made with government support under CA232347 and CA220801 awarded by the National Institutes of Health. The government has certain rights in the invention.

### REFERENCE TO A SEQUENCE LISTING

This application is being filed electronically via EFS-Web and includes an electronically submitted Sequence Listing in .txt format. The .txt file contains a sequence listing entitled “70258102132ST25.txt” created on Apr. 25, 2022 and is 18,669 bytes in size. The Sequence Listing contained in this .txt file is part of the specification and is hereby incorporated by reference herein in its entirety.

### BACKGROUND

The field of the invention relates to small molecule inhibitors of ubiquitin specific peptidase 22 (USP22) and the use thereof in treating diseases and disorders associated with USP22 biological activity. In particular, the field of the invention relates to small molecule inhibitors of the peptidase activity of USP22 which may be formulated as pharmaceutical compositions for treatment of cell proliferative diseases and disorders such as cancer.

The expression of ubiquitin specific peptidase 22 (USP22) is often increased in many, if not all types of human cancers. USP22 functions as a potential oncogene in tumorigenesis and progression in lung and colon cancer in part through diminishing the tumor suppressor p53 transcriptional activity and promoting cell cycle progression. Mice with genetic USP22 suppression in immune cells have better tumor rejection using multiple syngeneic tumor models including lung cancer, lymphoma, melanoma, and colon cancers. These results indicate that USP22 is an ideal therapeutic target in antitumor therapy because that, on one hand, inhibition of USP22 in tumor cells can directly induces their apoptosis and blocks cell cycle progression, on the other hand, USP22 suppression in immune cells enhances antitumor immunity.

### SUMMARY OF THE INVENTION

Disclosed herein are inhibitors of ubiquitin specific peptidase 22 (USP22) and uses for treating diseases and disorders thereof. One aspect of the technology provides for a method of treating a subject in need of treatment for a disease or disorder associated with ubiquitin specific peptidase 22 (USP22) activity, the method comprising administering to the subject an effective amount of a therapeutic agent that inhibits the biological activity of USP22. In some embodiments, the disease or disorder is a cell proliferative disease or disorder. In some embodiments, the disease or disorder is a cancer. In some embodiments, the cancer may be selected from the group consisting of lung cancer, gastric carcinoma, pancreatic cancer, melanoma, lymphoma, colon cancer, breast cancer, ovarian cancer, bladder cancer, prostate cancer, glioma, mesothelioma, neuroblastoma, mantle cell lymphoma, and acute myeloid leukemia.

Another aspect of the technology provides for a method of suppressing Treg cell activity in a subject in need thereof, the method comprising administering to the subject an effective amount of a therapeutic agent that inhibits the activity of USP22. In some embodiments, the subject has an infectious disease. In some embodiments, the subject has sudden acute respiratory syndrome coronavirus 2 (SARS-CoV2) infection.

Another aspect of the technology provides for a method for inhibiting ubiquitin specific peptidase activity (E. C. 3.4.19.12) of USP22 in a subject in need thereof, the method comprising administering to the subject an effective amount of a therapeutic agent that inhibits the biological activity of USP22.

For the disclosed methods, the therapeutic agent is an inhibitor of ubiquitin specific peptidase 22 (USP22). In some embodiments, the therapeutic agent comprises one or more compounds selected from Table Si. In some embodiments, the therapeutic agent is ii-anilino-7,8,9,10-tetrahydrobenzimidazo[1,2-b]isoquinoline-6-carbonitrile.

Pharmaceutical compositions comprising the therapeutic agents described herein and a suitable pharmaceutical carrier. In some embodiments, the therapeutic agent is ii-anilino-7,8,9,10-tetrahydrobenzimidazo[1,2-b]isoquinoline-6-carbonitrile. In some embodiments, the composition comprises an effective amount of the compound for inhibiting biological activity of USP22 when administered to a subject in need thereof. In some embodiments, the composition comprises an effective amount of the compound for suppressing Treg cell activity in a subject in need thereof. In some embodiments, the composition comprises an effective amount of the compound for inhibiting ubiquitin specific peptidase activity (E.C. 3.4.19.12) of USP22 in a subject in need thereof.

### BRIEF DESCRIPTION OF THE FIGURES

FIG. 1. Intratumoral T_{reg} cells have increased mRNA expression of Usp22 and Usp21. A-C, mRNA level of YFP+ sorted T_{reg} cells from control mice spleens, and tumor-challenged mice spleens and tumor cells. All mRNA values calculated relative to WT T_{reg} cell levels of unchallenged mice. spleens B16) Usp22: n=5-6, Usp21: n=3-5, Usp7: n=3-5. LLC1) Usp22: n=5-6, Usp21: n=3-6, Usp7: n=3-6. EG7) Usp22: n=4-5, Usp21 n=3-4, Usp7: n=3-7. D, mRNA level of Usp22, Usp21 and Foxp3 in CD4⁺CD25⁺CD127⁻T_{reg} cells isolated from human lung cancer tissues from patients relative to T_{reg} cells recovered from the cancer-adjacent healthy lung tissue isolated from the same patient. AHL: adjacent healthy lung; LTu: lung tumor. Usp22: n=8, Usp21: n=3, FoxP3: n=11. E, mRNA level of Usp21 and FoxP3 in T_{reg} cells isolated from human lung cancer patients. AHL: adjacent healthy lung; LTu: lung tumor n=9. A-C, Two-tailed unpaired t-test was done to determine statistical significance. D, Two-tailed paired t-test was performed to determine statistical significance of FoxP3 and Usp22 in Ltu vs. AHL. E, Linear regression was calculated for the correlation between Usp22 and FoxP3 within Ltu. All data are presented as mean±stdev. NS, not significant. *P<0.05, **P<0.01, ***P<0.001, ****P<0.0001.

FIG. 2: Tumor cell secreted TGF-β increases Usp22 and Usp21 level in iT_{reg} cells. A, USP mRNA level in iT_{reg} cells in control T cell media compared to addition of tumor cell treated media at 50/50 with T cell media for 24 hours. Usp22) Control: n=14, B16: n=10, LLC1: n=5, EG7: n=4. Usp21) Control: n=12, B16: n=8, LLC1: n=3, EG7: n=3. Usp7) Control: n=10, B16: n=7, LLC1: n=4, EG7: n=5 B, USP protein level in iT_{reg} cells in control T cell media compared to addition of tumor cell treated media at 50/50 with T cell media for 24 hours. C, USP mRNA level in iT_{reg} cells with the addition of a TGF-β inhibitor in tumor cell media (Usp22) Control: n=22, B16: n=15, B16+Inh: n=5, LLC1: n=10, LLC1+inh: n=5, EG7: n=7, EG7+inh: n=5. Usp21) Control: n=20, B16: n=13, B16+Inh: n=5, LLC1: n=8, LLC1+inh: n=4, EG7: n=7, EG7+inh: n=5. Usp7) Control: n=14, B16: n=10, B16+Inh: n=5, LLC1: n=8, LLC1+inh: n=3, EG7: n=8, EG7+inh: n=6. D, SMAD2, SMAD3, and SMAD4 binding capacity along the Usp22 promoter under TGFb inhibition. SMAD2: n=4-5; SMAD3 n=3; SMAD4: n=3. A-C, All mRNA values calculated relative to untreated WT iT_{reg} cells. A-D, Ordinary one-way ANOVA with multiple comparisons was performed to determine significance. All data are presented as mean±stdev. NS, not significant. *P<0.05, **P<0.01, ***P<0.001, ****P<0.0001.

FIG. 3: Usp22 and Usp21 are required for FOXP3 stability in nT_{reg} cells under environmental and metabolic stress found in the TME. All mRNA values calculated relative to unchallenged WT T_{reg} cells. A, nT_{reg} USP mRNA level in normoxic (21% O₂) verses hypoxic (1% O₂) conditions after 24 hours (n=6-13). B, FOXP3 MFI change in 22KO nT_{reg} cells relative to WT nT_{reg} cells after 72 hours in normoxic (21% O₂) verses hypoxic (1% O₂) conditions (n=5). C, nT_{reg} USP mRNA level after treatment with dMOG for 24 hours (n=6). D, USP mRNA level in nT_{reg} cells after exposure to glucose-restricted (0.5 mM) conditions after 24 hours relative to normal media (11 mM glucose) (n=7-18). E, Relative FOXP3 MFI change in nT_{reg} cells from control and cells cultured under low glucose conditions after 48 hours (n=3). F, nT_{reg} USP mRNA level under amino acid starvation for 24 hours (n=5-9). G, FOXP3 MFI stability in Usp22- or Usp21-null nT_{reg} cells cultured in normal media conditions verses amino acid starvation after 48 hours in (n=3). H, nT_{reg} USP mRNA level after treatment with 1 μM oligomycin A for 24 hours (n=5-7). I, nT_{reg} USP mRNA level after treatment with 250 nM Torin1 for 24 hours (n=5-7). A, C-D, F and H-I, Ordinary one-way ANOVA with multiple comparisons was performed to determine significance. B, E and G, Two-tailed unpaired t-test was performed to determine statistical significance. All data are presented as mean±stdev. NS, not significant. *P<0.05, **P<0.01, ***P<0.001, ****P<0.0001

FIG. 4: Loss of Usp22 and Usp21 in T_{reg} cells differentially impairs FoxP3 expression and cell function. A, Usp22 and Usp21 levels in WT, 21KO, 22KO and dKO mice (n=5-8). All mRNA values calculated relative to WT T_{reg} cells. B, Mice weights over a 2-month period (n=2-9). C, Peripheral activation of CD4+ and CD8+ T cells as measured by CD44^{hi}CD62L^{lo} expression (n=7-9). D, Representative histogram (left) and quantification (right) of FOXP3 MFI in splenic T_{reg} cells of WT and KO animals (n=6-8). E, Heat map of T_{reg} cell signature genes (*significance is adjusted P<0.01) in 21KO (n=2), 22KO (n=3), and dKO (n=3) versus WT (n=3) mice. F, Venn Diagram of DEGs (adj. p<0.01) between 22KO, 21KO and dKO (n=2-3). G, Normalized enrichment scores from gene set enrichment analysis (False Discovery Rate, FDR<25%) from the hallmark gene set in the molecular signatures database comparing the gene set generated from RNA sequencing of Wt, 21KO, 22KO, and dKO mice (n=2-3). A-C, Two-way ANOVA with multiple comparisons between rows was performed to determine statistical significance. D, One-way ANOVA with multiple comparisons between rows was performed to determine statistical significance. All data are presented as mean±stdev. NS, not significant. *P<0.05, **P<0.01, ***P<0.001, ****P<0.0001.

FIG. 5: Deletion of Usp21 and Usp22 in T_{reg} cells synergize to enhance antitumor immunity A, Tumor growth curve of B16 cells subcutaneously injected in the flank of WT, 21KO, 22KO and dKO mice (n=13-14). B, Percent activation as defined by CD44^{hi}CD62L^{lo} of CD4⁺ and CD8⁺ T cells in the spleens of B16 challenged mice (n=5-6). C, Percent IFN-γ and Granzyme B (GZMB) production of peripheral CD8+ T cells (n=3). D, FOXP3 MFI of peripheral T_{reg} cells relative to WT (n=7-9). E, PD-1 MFI of peripheral T_{reg} cells relative to WT (n=3). F, GITR MFI of peripheral T_{reg} cells relative to WT (n=6-8). G, LAG3 MFI of peripheral T_{reg} cells relative to WT (n=3). H, Representative flow cytometry plot and graphical representation of % infiltration of CD4+ and CD8+ T cells within the tumor (n=5-6). I-J, Percentage IFN-γ and GZMB production of intratumoral CD8⁺ and CD4⁺ cells (n=5-6). K, Representative FOXP3+ percentage of CD4+ cells relative to WT in itT_{reg} cells (n=6). L, Representative flow plot (left) and quantitative representation of FOXP3 MFI within tumor T_{reg} cells relative to WT (n=6-9). M, Tumor growth curve of B16 cells treated with TGFβ shRNA or scramble control shRNA subcutaneously injected in the flank of WT and dKO mice (n=3-4). A-C, H-J, and M Two-way ANOVA with multiple comparisons between rows was performed to determine statistical significance. All data are presented as mean±stdev. NS, not significant. *P<0.05, **P<0.01, ***P<0.001, ****P<0.0001. D-G and K and L, One-way ANOVA with multiple comparisons between rows was performed to determine statistical significance. All data are presented as mean±stdev. NS, not significant. *P<0.05, **P<0.01, ***P<0.001, ****P<0.0001.

FIG. 6: Usp22 inhibitor administration enhances antitumor immunity. A, Structure of compound CS30 (Usp22i-S02). B, FOXP3 MFI in WT and 22KO of T_{reg} cells after treatment with 20 mg/kg of Usp22i-S02 in vivo (n=3). C, Representative flow cytometry plot of FOXP3+CD25+ MFI of CD4+ peripheral cells of mice treated with 20 mg/kg of Usp22i-S02 relative to control (n=5). D, Graphical representation of Foxp3 MFI upon Usp22i-S02 administration (n=5). E, Tumor growth curve of LLC1 cells subcutaneously injected in the flank of WT mice with or without the addition of 20 mg/kg/time of the Usp22 inhibitor starting at day 15, in 100 μL of oil (n=4). F-G, Representative flow cytometry plot and graphical representation of % infiltration of CD4+ and CD8+ T cells within the tumor (n=4). H, Representative histogram plot and graphical representation of itT_{reg} Foxp3 MFI (n=4). I, MFI of itT_{reg} suppressive markers (n=3-4). J, Percent Foxp3+IFNg+ itT_{reg} cells in control and Usp22-S02 treated mice (n=3-4). B, D-E, G, H-I Two-way ANOVA with multiple comparisons between rows was performed to determine statistical significance. J, Unpaired two tailed T test was performed to determine significance. All data are presented as mean±stdev. NS, not significant. *P<0.05, **P<0.01, ***P<0.001, ****P<0.0001.

FIG. 7: Intratumoral T_{reg} cells have increased Foxp3 and activation markers. A, Representative CD4+ FOXP3+ percentage by flow cytometry of cells from non-tumor challenged controls and B16−, LLC1−, and EG7− challenged mice. B16: n=3-4, EG7: n=2-6, and LLC1: n=5-6. B, Representative overlay of FOXP3 MFI in tumor and spleen of CD45+CD4+ FOXP3+(T_{reg}) cells of control and tumor-challenged mice. C, Quantification of FOXP3 MFI in control spleen and tumor-challenged spleen and tumor T_{reg} cells (n=4-11). D-G, MFI of T_{reg} cell-associated markers under control T_{reg} cells isolated from the spleen and splenic and tumor T_{reg} cells from B16, EG7, or LLC1 challenged animals. CD25: n=4-11; GITR: n=3-11; CTLA-4: n=4-11; PD-1: n=4-11. All MFI values calculated relative to WT T_{reg} cell levels of non-challenged mice spleens. C-G, Two-tailed unpaired t-test was performed to determine statistical significance. All data are presented as mean±stdev. NS, not significant. *P<0.05, **P<0.01, ***P<0.001, ****P<0.0001.

FIG. 8: TGF-β induces expression of Usp22 and Usp21 in T_{reg} cells. A, Visual representation of Tumor Conditioned Media (TCM) experiments. B, iT_{reg} USP mRNA level under TGF-β induction post-polarization. Usp22: n=18-19; Usp21: n=7-8; Usp7: 4-5. C, iT_{reg} USP mRNA level under TGF-β with and without a TGF-β inhibitor. Usp22: n=3-11; Usp21: n=8-18; Usp7: 3-8. D, T_{reg} FoxP3 mRNA level with TGF-β induction (n=10). B-D, All mRNA values calculated relative to WT untreated iT_{reg} cells. E, TGF-β level in B16, LLC1 and EG7 tumor conditioned media (n=3). F-H, Correlation between USP induction and TGF-β level in the tumor conditioned media. Usp22: n=3-10; Usp21: n=3-8; Usp7: n=3-6. I, TGF-β mRNA level of control or TGF-β shRNA treated B16 cells (n=3). J, mRNA level of Usp22 in sorted itT_{reg} cells from mice injected with shRNA treated B 16 cells (n=4). B, D, and I, Two-tailed unpaired t-test was performed to determine statistical significance. C and E Ordinary one-way Anova with multiple comparisons between groups was performed to determine statistical significance. All data are presented as mean±stdev. NS, not significant. *P<0.05, **P<0.01, ***P<0.001, ****P<0.0001.

FIG. 9: SMAD3 and SMAD4 bind to conserved SBE on the Usp22 promoter while Usp21 is upregulated by non-canonical TGF-β signaling. A, Usp22 promoter region overlaid with plausible SMAD binding elements (SBE) and placement of primers created for ChIP. B and C, Binding of SMAD2, SMAD3 and SMAD4 along the Usp22 and Usp21 promoter regions using ChIP-qPCR Usp22: n=2-7; Usp21: n=1-2. D-F, Representative flow plot and graphical representation of Foxp3 MFI and percentage in WT and Usp21-null iT_{reg} cells polarized in 5 ng/μl of TGF-β (n=3). G, iT_{reg} Usp21 mRNA level under TGF-β with and without a TGF-β non-canonical pathway p38 kinase inhibitor (p38i) (n=4). E and F, Two-tailed unpaired t-test was performed to determine statistical significance. G, Ordinary one-way Anova with multiple comparisons between groups was performed to determine statistical significance. All data are presented as mean±stdev. NS, not significant. *P<0.05, **P<0.01, ***P<0.001, ****P<0.0001.

FIG. 10: USP22 reciprocally enhances TGF-β signaling through SMAD protein stabilization in positive feedback loop. A, Representative protein level of SMAD2, SMAD3, and SMAD4 in USP22 WT and KO iT_{reg} cells. B, mRNA level of SMAD2, SMAD3, and SMAD4 in USP22 WT and KO iT_{reg} cells (n=3). All mRNA values calculated relative to unchallenged WT iT_{reg} cells. Two-tailed unpaired t-test was performed to determine statistical significance. All data are presented as mean±stdev. NS, not significant. *P<0.05, **P<0.01, ***P<0.001, ****P<0.0001. C, USP22 endogenous IP with SMAD 2, SMAD 3, and SMAD 4 proteins within iT_{reg} cells. D-F, Overexpression DUB assay IP in 293T cells of USP22 with SMAD2, SMAD3, and SMAD4. G, SMAD2 and SMAD4 protein degradation in WT and KO iT_{reg} cells under cycloheximide treatment for 2, 4 and 6 hours. H, SMAD2 and SMAD4 protein degradation in WT and KO iT_{reg} cells under cycloheximide treatment with or without MG132 protease inhibitor at 4 hours.

FIG. 11: HIF-α and the AMPK/mTOR balance modulates T_{reg} cell FoxP3 stability through USP22 and USP21. All mRNA values calculated relative to unchallenged WT T_{reg} cells. A, iT_{reg} USP mRNA level in normoxic and hypoxic conditions after 4 hours (n=4-5). B, iT_{reg} USP protein level in normoxic and hypoxic conditions after 24 hours. C, Visual representation of stability assay calculations of Foxp3 MFI level. %02 is the percentage of oxygen, Glu is glucose, and AA is amino acids. One variable was changed at a time, the others kept at baseline control. D, iT_{reg} cell USP mRNA level after treatment with DMOG for 24 hours (n=6). E, nT_{reg} cell Foxp3 MFI change treated with DMOG for 48 hours relative to untreated nT_{reg} (n=9-10). F, Foxp3 MFI change of iT_{reg} after 72 h in hypoxia compared to untreated cells (n=3). G, Foxp3 MFI change of iT_{reg} treated with DMOG for 72 hours relative to untreated iT_{reg} cells (n=4) H, iT_{reg} cell USP mRNA level under low glucose conditions after 24 hours (n=3-8). I, iT_{reg} cell USP protein level under low glucose conditions after 24 hours. J, iT_{reg} Foxp3 MFI change low glucose conditions after 72 hours relative to complete media (n=7). K, iT_{reg} cell USP mRNA level in amino acid starvation relative to complete media after 24 hours (n=6). L, Foxp3 MFI change of iT_{reg} cells under amino acid starvation for 72 hours relative to untreated iT_{reg} cells (n=6). M, iT_{reg} cell USP mRNA level after treatment with 1 μM oligomycin for 24 hours (n=6). N, iT_{reg} USP mRNA level after treatment with 250 nM Torin1 for 24 hours (n=5-9). A, D, H, and K-M, Ordinary one-way Anova with multiple comparisons between groups was performed to determine statistical significance. E-G and J, Two-tailed unpaired t-test was performed to determine statistical significance. All data are presented as mean±stdev. NS, not significant. *P<0.05, **P<0.01, ***P<0001 ****P<0.0001.

FIG. 12: Loss of Usp22 and Usp21 in T_{reg} cells differentially alter T_{reg} metabolic pathways. A, T and B cell percentages in peripheral organs of KO and WT animals (n=5-9). B, Percent of CD4 and CD8 T cells in peripheral organs of CD45+ cells?(n=4-10). C E, Heat map of metabolic pathways (significance is noted by * adjusted P<0.01) in U21KO (n=2), U22KO (n=3), and dKO (n=3) versus WT (n=3) mice. Genes chosen based on differential expression (adj. p<0.01) in the dKO mice. F, Basal mitochondrial OCR and G, Basal ECAR of 21KO (n=5), 22KO (n=5) and dKO (n=4-5) relative to WT (n=5) nT_{reg} cells. A-B, Two-way ANOVA with Sidak's multiple comparisons between rows was performed to determine statistical significance. F-G, One-way ANOVA with Tukey's multiple comparisons between rows was performed to determine statistical significance. All data are presented as mean±stdev. NS, not significant. *P<0.05, **P<0.01, ***P<0.001, ****P<0.0001.

FIG. 13: Usp21-deletion alters cell. A, Normalized enrichment scores from gene set enrichment analysis (False Discovery Rate, FDR<1%) from the hallmark gene set in the molecular signatures database comparing the gene set generated from RNA sequencing of 22KO and dKO mice (n=3). B, Representative graph of percent Ki67 positive cells within the CD4+ Foxp3+ T_{reg} compartment (n=7). One-way ANOVA with Tukey's multiple comparisons between rows was performed to determine statistical significance. All data are presented as mean±stdev. NS, not significant. *P<0.05, **P<0.01, ***P<0.001, ****P<0.0001.

FIG. 14. Development and validation of Usp22-specific inhibitor through structure-based hierarchical virtual screening. A, Flowchart of structure-based virtual screening. B, The overall conformation of USP22-m (USP22 model generated using SWISS MODEL) is represented by cartoon which are colored by conservation using the color-code bar. Catalytic centre of USP22 was defined as docking position. C, Ramachandran plot statistics of USP22-m generated by PROCHEK progress (left). The Displacement of the catalytic centre loop in USP22-md (the MD optimized model) compared to UBP8 (PDB: 3MHS) and USP22-m (right). D, S02 displayed in green stick binding in the pocket of USP22-md structure (left). Ligplot showing hydrogen bonding and hydrophobic contacts of S02 with USP22-md (middle). The best ranked position of S02 (shown in green) in the binding pocket of USP22-md is presented, generated by docking. E, Individual energy contributions of amino acid residues after MD simulations and PBSA calculations. F-G, Binding free energies of compound S02 to USP22 Model.

FIG. 15: Usp22i-S02 halts Usp22-mediated Foxp3 deubiquitination. A, Graphical representation of Foxp3 MFI change in WT versus 22KO iT_{reg} cells treated with various doses of Usp22i-S02 (n=3). B, Representative histogram of Foxp3 MFI level in iT_{reg} cells as Usp22 inhibitor concentration increases from 0-20 μg/mL. C, Cell survival of iT_{reg} cells treated with various doses of Usp22i-S02 (n=3). D, FOXP3 and USP22 protein level in WT and 22KO mice treated with 10 μg/mL Usp22i-S02. E-F, Graphical and representative data of Foxp3 MFI of human T_{reg} cells treated with various doses of Usp22i-S02 (n=3). G, Graphical representation of Foxp3 MFI in WT, Usp21-null, and Usp22-null T_{reg} cells treated with Usp22i-S02 at 10 μg/mL (n=3). H, FOXP3 and USP22 protein degradation of cycloheximide (10 μg/mL) treated iT_{reg} cells with or without the addition of 10 μg/mL of Usp22i-S02. I-J, Endogenous DUB assay IP in iT_{reg} cells of USP22 with FOXP3 under increasing concentrations of Usp22i-S02. K, Foxp3 mRNA level in iT_{reg} cells as Usp22 inhibitor concentration increases from 0-20 μg/mL (n=3). L, FOXP3 and USP22 level in WT iT_{reg} cells with or without 20 μg/mL Usp22 inhibitor treated with 20 μM MG132. M, Graphical representation of the percentage of decrease of Foxp3 MFI in either WT or Usp22-null nT_{reg} cells placed in low glucose conditions with or without the addition of 10 μg/mL of Usp22i-S02 (n=7-8). N, Graphical representation of the percentage of decrease of Foxp3 MFI in WT nT_{reg} cells placed in hypoxic or low amino acid conditions with or without the addition of 10 μg/mL of Usp22i-S02 (n=3-5). G, Two-tailed unpaired t-test comparing within groups was performed to determine statistical significance. K, One-way ANOVA with Dunnet's multiple comparisons between rows relative to control was performed to determine statistical significance. M-N, Two-way ANOVA with Sidak's multiple comparisons between rows was performed to determine statistical significance. All data are presented as mean±stdev. NS, not significant. *P<0.05, **P<0.01, ***P<0.001, ****P<0.0001.

FIG. 16: Usp22i-S02 has little effect on naïve mice, yet enhances anti-tumor immunity in LLC1-challenged mice. A, Body weight of Usp22i-S02 treated mice verses DMSO treated controls over the course of treatment (n=4). A-F. Injections were twice a day for 3 consecutive days at 10 mg/kg (n=4). B, Percent of cell populations in naïve mice treated with Usp22i-S02 relative to DMSO control (n=3-4). C, Percent of KI-67+ cells in various compartments in naïve mice treated with Usp22i-S02 relative to DMSO control (n=3-4). D, Percent CD44^{hi}CD62L¹⁰ in T cell populations gated on CD45+ cells in naïve mice treated with Usp22i-S02 relative to DMSO control (n=3). E, Percent Annexin+PI+ T cells gates on CD45+ cells in naïve mice treated with Usp22i-S02 relative to DMSO control (n=4). F-H, Organ toxicity panel (VetScan VS2 Comprehensive Diagnostic Rotor lot 1061AA2) of naïve mice treated with Usp22i-S02 relative to DMSO control (n=2-3). I, Growth curve of subcutaneously injected LLC1 in WT mice treated with Usp22i-S02 at 10 mg/kg (n=5-10). I-Q, Injections were twice a day for 5 consecutive days at 10 mg/kg. J, Weights of resected tumors at day 16 from I (n=10). K-L, Representative flow plot and graphical representation of infiltrating T cells gated on CD45+ cells (n=5). M-P, Characterization of intratumoral CD8+ T cells from mice treated with Usp22i-S02 (n=3-5). Q, Percent of intratumoral T_{reg} cells from mice treated with Usp22i-S02 verses DMSO control, gated on CD4+Foxp3+ from mice (n=5). A-E, I and L, Two-way ANOVA with Sidaks's multiple comparisons between rows relative to control was performed to determine statistical significance. F-H, J, and M-Q, Two-tailed unpaired t-test was performed to determine statistical significance. All data are presented as mean±stdev. NS, not significant. *P<0.05, **P<0.01, ***P<0.001, ****P<0.0001.

FIG. 17: Usp22i-S02 inhibits tumor growth in vitro and in vivo. A, In vitro counts of LLC1 after treatment with Usp22i-S02 under various concentrations for 24 hours (n=2). B, In vitro viability of LLC1 after treatment with Usp22i-S02 under various concentrations for 24 hours (n=2). C, In vitro relative growth of LLC1 cells treated with 10 ug/ml of Usp22i-S02 relative to DMSO treated control for 7 days via OD600 (n=4). D, Growth curve of 1 million subcutaneously injected LLC1 cells into RAG−/− mice treated for 3 days Usp22i-S02 relative to DMSO control injections at day 15 of tumor growth (n=4). C-D, Two-way ANOVA with Sidaks's multiple comparisons between rows relative to control was performed to determine statistical significance. All data are presented as mean±stdev. NS, not significant. *P<0.05, **P<0.01, ***P<0.001, ****P<0.0001.

FIG. 18: TME-specific factors can drive increased levels of Usp22 and Usp21 potentially through modulation of TGF-β signaling, HIF1a, AMPK, and mTOR activity to render T_{reg} cells more stable in the tumor microenvironment.

### DETAILED DESCRIPTION

Disclosed herein are inhibitors of ubiquitin specific peptidase 22 (USP22) and uses for treating diseases and disorders thereof. Computer-based and biological approaches were used to identify small molecule specific inhibitors. As demonstrated in the Examples, treatment of regulatory T cells (Tregs), both mouse and human, with inhibitors of USP22 significantly reduced the protein expression of FoxP3, a substrate of USP22. In contrast, treatment did not further inhibit FoxP3 expression in USP22-null Tregs, indicating that the inhibitors of USP22 may be a highly specific inhibitor of USP22. In addition, treatment inhibited USP22 activity in lung cancer cells and consequently suppressed lung cancer cell growth. More importantly, treatment of lung cancer-bearing mice largely diminished the tumor mass. These results indicate that inhibitors of USP22 can be used as a potent drug in antitumor therapy. In addition, the fact that suppression of USP22 diminishes T_{reg} suppressive functions, also allows for these inhibitors to be used to treat diseases associated to immune deficiency as well as to boost the immune response to combat infectious diseases such as SARS-CoV2 infection.

The present invention is described herein using several definitions, as set forth below and throughout the application.

### Definitions

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.).

It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

A “subject in need thereof” as utilized herein may refer to a subject in need of treatment for a disease or disorder associated with ubiquitin specific peptidase 22 (USP22) activity and/or expression. A subject in need thereof may include a subject having a cancer that is characterized by the activity and/or expression of USP22. The disclosed compounds, pharmaceutical compositions, and methods may be utilized to treat diseases and disorders associated with USP22 activity and/or expression.

In some embodiments, a subject in need thereof may include a subject having a cancer that is treated by administering a therapeutic agent that inhibits the biological activity of USP22, and/or that inhibits dissemination of cancer cells.

The disclosed compounds, pharmaceutical compositions, and methods may be utilized to treat diseases and disorders associated with USP22 activity and/or expression which may include cell proliferative diseases and diseases and disorders such as cancers. Suitable cancers for treatment by the disclosed compounds, pharmaceutical compositions, and methods may include, but are not limited to lung cancer, gastric carcinoma, pancreatic cancer, melanoma, lymphoma, colon cancer, breast cancer, ovarian cancer, bladder cancer, prostate cancer, glioma, mesothelioma, neuroblastoma, mantle cell lymphoma, and acute myeloid leukemia.

In some embodiments, a subject in need thereof may include a subject in need of treatment of infection. In some embodiments, the infection is a viral infection, such as an infection by a corona virus. In some embodiments, the subject in need thereof is in need of a treatment for infection by sudden acute respiratory syndrome coronavirus 2 (SARS-CoV2) and COVID. In some embodiments, a subject in need thereof may refer to a subject in need of augmenting the immune response to an infection. In some embodiments, a subject in need thereof may refer to a subject in need of augmenting the immune response to sudden acute respiratory syndrome coronavirus 2 (SARS-CoV2) infection.

The disclosed compounds, pharmaceutical compositions, and methods may be utilized to treat diseases and disorders associated with USP22 activity and/or expression which may include infections and diseases and disorders such as respiratory infections, including sudden acute respiratory syndrome coronavirus 2 (SARS-CoV2) infection.

The term “subject” may be used interchangeably with the terms “individual” and “patient” and includes human and non-human mammalian subjects.

The disclosed compounds may be utilized to modulate the biological activity of USP22, including modulating the peptidase activity of USP22. The term “modulate” should be interpreted broadly to include “inhibiting” USP22 biological activity including peptidase activity.

Ubiquitin specific peptidase (USP22) refers to the protein also referred to by the name ubiquitin carboxyl-terminal hydrolase 22. USP22 has been shown to have enzyme activities that include catalyzing the thiol-dependent hydrolysis of ester, thioester, amide, peptide and isopeptide bonds formed by the C-terminal glycine of ubiquitin. USP22 has ENZYME entry: EC 3.4.19.12. The compounds disclosed herein may inhibit one or more of the activities of USP22 accordingly.

Human USP22 is known to have two isoforms and the disclosed compounds may inhibit one or more activities of isoform 1 and/or isoform 2.

Human USP22 Isoform 1 has the following amino acid sequence:

```
(SEQ ID NO: 1)
                10 20 30 40 50
MVSRPEPEGE AMDAELAVAP PGCSHLGSFK VDNWKQNLRA IYQCFVWSGT
                60 70 80 90 100
AEARKRKAKS CICHVCGVHL NRLHSCLYCV FFGCFTKKHI HEHAKAKRHN
              110 120 130 140 150
LAIDLMYGGI YCFLCQDYIY DKDMEIIAKE EQRKAWKMQG VGEKFSTWEP
              160 170 180 190 200
TKRELELLKH NPKRRKITSN CTIGLRGLIN LGNTCFMNCI VQALTHTPLL
              210 220 230 240 250
RDFFLSDRHR CEMQSPSSCL VCEMSSLFQE FYSGHRSPHI PYKLLHLVWT
              260 270 280 290 300
HARHLAGYEQ QDAHEFLIAA LDVLHRHCKG DDNGKKANNP NHCNCIIDQI
              310 320 330 340 350
FTGGLQSDVT CQVCHGVSTT IDPFWDISLD LPGSSTPFWP LSPGSEGNVV
              360 370 380 390 400
NGESHVSGTT TLTDCLRRFT RPEHLGSSAK IKCSGCHSYQ ESTKQLTMKK
              410 420 430 440 450
LPIVACFHLK RFEHSAKLRR KITTYVSFPL ELDMTPFMAS SKESRMNGQY
              460 470 480 490 500
QQPTDSLNND NKYSLFAVVN HQGTLESGHY TSFIRQHKDQ WFKCDDAIIT
              510 520
KASIKDVLDS EGYLLFYHKQ FLEYE
```

Isoform 2 has the following sequence:

```
(SEQ ID NO: 2)
                10 20 30 40 50
MAPGWPSLSA GSRQEAPQLA AGGSAYQAVG RQFQPRATAL QGPSQAKSCI
                60 70 80 90 100
CHVCGVHLNR LHSCLYCVFF GCFTKKHIHE HAKAKRHNLA IDLMYGGIYC
              110 120 130 140 150
FLCQDYIYDK DMEIIAKEEQ RKAWKMQGVG EKFSTWEPTK RELELLKHNP
              160 170 180 190 200
KRRKITSNCT IGLRGLINLG NTCFMNCIVQ ALTHTPLLRD FFLSDRHRCE
              210 220 230 240 250
MQSPSSCLVC EMSSLFQEFY SGHRSPHIPY KLLHLVWTHA RHLAGYEQQD
              260 270 280 290 300
AHEFLIAALD VLHRHCKGDD NGKKANNPNH CNCIIDQIFT GGLQSDVTCQ
              310 320 330 340 350
VCHGVSTTID PFWDISLDLP GSSTPFWPLS PGSEGNVVNG ESHVSGTTTL
              360 370 380 390 400
TDCLRRFTRP EHLGSSAKIK CSGCHSYQES TKQLTMKKLP IVACFHLKRF
              410 420 430 440 450
EHSAKLRRKI TTYVSFPLEL DMTPFMASSK ESRMNGQYQQ PTDSLNNDNK
              460 470 480 490 500
YSLFAVVNHQ GTLESGHYTS FIRQHKDQWF KCDDAIITKA SIKDVLDSEG
              510
YLLFYHKQFL EYE
```

### Pharmaceutical Compositions

The compounds employed in the compositions and methods disclosed herein may be administered as pharmaceutical compositions and, therefore, pharmaceutical compositions incorporating the compounds are considered to be embodiments of the compositions disclosed herein. Such compositions may take any physical form which is pharmaceutically acceptable; illustratively, they can be orally administered pharmaceutical compositions. Such pharmaceutical compositions contain an effective amount of a disclosed compound, which effective amount is related to the daily dose of the compound to be administered. Each dosage unit may contain the daily dose of a given compound or each dosage unit may contain a fraction of the daily dose, such as one-half or one-third of the dose. The amount of each compound to be contained in each dosage unit can depend, in part, on the identity of the particular compound chosen for the therapy and other factors, such as the indication for which it is given. The pharmaceutical compositions disclosed herein may be formulated so as to provide quick, sustained, or delayed release of the active ingredient after administration to the patient by employing well known procedures.

The compounds for use according to the methods of disclosed herein may be administered as a single compound or a combination of compounds. For example, a compound that inhibits the biological activity of ubiquitin specific peptidase 22 (USP22) may be administered as a single compound or in combination with another compound inhibits the biological activity of USP22 or that has a different pharmacological activity.

As indicated above, pharmaceutically acceptable salts of the compounds are contemplated and also may be utilized in the disclosed methods. The term “pharmaceutically acceptable salt” as used herein, refers to salts of the compounds, which are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by the skilled reader that most or all of the compounds as disclosed herein are capable of forming salts and that the salt forms of pharmaceuticals are commonly used, often because they are more readily crystallized and purified than are the free acids or bases.

Acids commonly employed to form acid addition salts may include inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of suitable pharmaceutically acceptable salts may include the sulfate, pyrosulfate, bisulfate, sulfite, bisulfate, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleat-, butyne-.1,4-dioate, hexyne-1,6-dioate, benzoate, chlorobenzoate, methylbenzoate, hydroxybenzoate, methoxybenzoate, phthalate, xylenesulfonate, phenylacetate, phenylpropionate, phenylbutyrate, citrate, lactate, α-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Bases useful in preparing such salts include sodium hydroxide, potassium hydroxide, ammonium hydroxide, potassium carbonate, sodium carbonate, sodium bicarbonate, potassium bicarbonate, calcium hydroxide, calcium carbonate, and the like.

The particular counter-ion forming a part of any salt of a compound disclosed herein is may not be critical to the activity of the compound, so long as the salt as a whole is pharmacologically acceptable and as long as the counter-ion does not contribute undesired qualities to the salt as a whole. Undesired qualities may include undesirably solubility or toxicity.

Pharmaceutically acceptable esters and amides of the compounds can also be employed in the compositions and methods disclosed herein. Examples of suitable esters include alkyl, aryl, and aralkyl esters, such as methyl esters, ethyl esters, propyl esters, dodecyl esters, benzyl esters, and the like. Examples of suitable amides include unsubstituted amides, monosubstituted amides, and disubstituted amides, such as methyl amide, dimethyl amide, methyl ethyl amide, and the like.

In addition, the methods disclosed herein may be practiced using solvate forms of the compounds or salts, esters, and/or amides, thereof. Solvate forms may include ethanol solvates, hydrates, and the like.

The pharmaceutical compositions may be utilized in methods of treating a disease or disorder associated with the biological activity of ubiquitin specific peptidase 22 (USP22). As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration.

As used herein the term “effective amount” refers to the amount or dose of the compound, upon single or multiple dose administration to the subject, which provides the desired effect in the subject under diagnosis or treatment. The disclosed methods may include administering an effective amount of the disclosed compounds (e.g., as present in a pharmaceutical composition) for treating a disease or disorder associated with biological activity of ubiquitin specific peptidase 22 (USP22).

An effective amount can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

A typical daily dose may contain from about 0.01 mg/kg to about 100 mg/kg (such as from about 0.05 mg/kg to about 50 mg/kg and/or from about 0.1 mg/kg to about 25 mg/kg) of each compound used in the present method of treatment.

Compositions can be formulated in a unit dosage form, each dosage containing from about 1 to about 500 mg of each compound individually or in a single unit dosage form, such as from about 5 to about 300 mg, from about 10 to about 100 mg, and/or about 25 mg. The term “unit dosage form” refers to a physically discrete unit suitable as unitary dosages for a patient, each unit containing a predetermined quantity of active material calculated to produce the desired therapeutic effect, in association with a suitable pharmaceutical carrier, diluent, or excipient.

Oral administration is an illustrative route of administering the compounds employed in the compositions and methods disclosed herein. Other illustrative routes of administration include transdermal, percutaneous, intravenous, intramuscular, intranasal, buccal, intrathecal, intracerebral, or intrarectal routes. The route of administration may be varied in any way, limited by the physical properties of the compounds being employed and the convenience of the subject and the caregiver.

As one skilled in the art will appreciate, suitable formulations include those that are suitable for more than one route of administration. For example, the formulation can be one that is suitable for both intrathecal and intracerebral administration. Alternatively, suitable formulations include those that are suitable for only one route of administration as well as those that are suitable for one or more routes of administration, but not suitable for one or more other routes of administration. For example, the formulation can be one that is suitable for oral, transdermal, percutaneous, intravenous, intramuscular, intranasal, buccal, and/or intrathecal administration but not suitable for intracerebral administration.

The inert ingredients and manner of formulation of the pharmaceutical compositions are conventional. The usual methods of formulation used in pharmaceutical science may be used here. All of the usual types of compositions may be used, including tablets, chewable tablets, capsules, solutions, parenteral solutions, intranasal sprays or powders, troches, suppositories, transdermal patches, and suspensions. In general, compositions contain from about 0.5% to about 50% of the compound in total, depending on the desired doses and the type of composition to be used. The amount of the compound, however, is best defined as the “effective amount”, that is, the amount of the compound which provides the desired dose to the patient in need of such treatment. The activity of the compounds employed in the compositions and methods disclosed herein are not believed to depend greatly on the nature of the composition, and, therefore, the compositions can be chosen and formulated primarily or solely for convenience and economy.

Capsules are prepared by mixing the compound with a suitable diluent and filling the proper amount of the mixture in capsules. The usual diluents include inert powdered substances (such as starches), powdered cellulose (especially crystalline and microcrystalline cellulose), sugars (such as fructose, mannitol and sucrose), grain flours, and similar edible powders.

Tablets are prepared by direct compression, by wet granulation, or by dry granulation. Their formulations usually incorporate diluents, binders, lubricants, and disintegrators (in addition to the compounds). Typical diluents include, for example, various types of starch, lactose, mannitol, kaolin, calcium phosphate or sulfate, inorganic salts (such as sodium chloride), and powdered sugar. Powdered cellulose derivatives can also be used. Typical tablet binders include substances such as starch, gelatin, and sugars (e.g., lactose, fructose, glucose, and the like). Natural and synthetic gums can also be used, including acacia, alginates, methylcellulose, polyvinylpyrrolidine, and the like. Polyethylene glycol, ethylcellulose, and waxes can also serve as binders.

Tablets can be coated with sugar, e.g., as a flavor enhancer and sealant. The compounds also may be formulated as chewable tablets, by using large amounts of pleasant-tasting substances, such as mannitol, in the formulation. Instantly dissolving tablet-like formulations can also be employed, for example, to assure that the patient consumes the dosage form and to avoid the difficulty that some patients experience in swallowing solid objects.

A lubricant can be used in the tablet formulation to prevent the tablet and punches from sticking in the die. The lubricant can be chosen from such slippery solids as talc, magnesium and calcium stearate, stearic acid, and hydrogenated vegetable oils.

Tablets can also contain disintegrators. Disintegrators are substances that swell when wetted to break up the tablet and release the compound. They include starches, clays, celluloses, algins, and gums. As further illustration, corn and potato starches, methylcellulose, agar, bentonite, wood cellulose, powdered natural sponge, cation-exchange resins, alginic acid, guar gum, citrus pulp, sodium lauryl sulfate, and carboxymethylcellulose can be used.

Compositions can be formulated as enteric formulations, for example, to protect the active ingredient from the strongly acid contents of the stomach. Such formulations can be created by coating a solid dosage form with a film of a polymer which is insoluble in acid environments and soluble in basic environments. Illustrative films include cellulose acetate phthalate, polyvinyl acetate phthalate, hydroxypropyl methylcellulose phthalate, and hydroxypropyl methylcellulose acetate succinate.

Transdermal patches can also be used to deliver the compounds. Transdermal patches can include a resinous composition in which the compound will dissolve or partially dissolve; and a film which protects the composition, and which holds the resinous composition in contact with the skin. Other, more complicated patch compositions can also be used, such as those having a membrane pierced with a plurality of pores through which the drugs are pumped by osmotic action.

As one skilled in the art will also appreciate, the formulation can be prepared with materials (e.g., actives excipients, carriers (such as cyclodextrins), diluents, etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

### Inhibitors of Ubiquitin Specific Peptidase 22 (USP22) Uses Thereof

Disclosed are compounds, pharmaceutical compositions comprising the compounds, and methods of using the compounds and pharmaceutical compositions for treating a subject having or at risk for developing a disease or disorder associated with ubiquitin specific peptidase 22 (USP22) biological activity. The disclosed compounds may inhibit the biological activity of USP22. As such, the disclosed compounds and pharmaceutical compositions may be utilized in methods for treating a subject having or at risk for developing a disease or disorder that is associated with USP22 activity which may be cell proliferative diseases and disorders, such as cancer, or an infection associated disease or disorder, such as sudden acute respiratory syndrome, such as SARS-CoV2.

In some embodiments, the disclosed methods include treating a subject in need of treatment for a disease or disorder associated with ubiquitin specific peptidase 22 (USP22) activity. In the disclosed methods, the subject may be administered an effective amount of a therapeutic agent that inhibits the biological activity of USP22.

The disclosed methods may be performed in order to treat a cell proliferative disease or disorder, which may include cancer. Suitable cancers that may be treated by the disclosed methods may include, but are not limited to, lung cancer, gastric carcinoma, pancreatic cancer, melanoma, lymphoma, colon cancer, breast cancer, ovarian cancer, bladder cancer, prostate cancer, glioma, mesothelioma, neuroblastoma, mantle cell lymphoma, and acute myeloid leukemia.

In some embodiments, the disclosed methods may be performed in order to treat lung cancer, for example, non-small cell lung cancer (NSCLC).

In some embodiments, the disclosed methods may be performed in order to treat skin cancer, for example, melanoma.

In the disclosed methods, a subject in need thereof typically is administered a therapeutic agent that inhibits the biological activity of ubiquitin specific peptidase 22 (USP22). In some embodiments, the therapeutic agent inhibits ubiquitin specific peptidase activity (E.C.: 3.4.19.12) of USP22.

Suitable therapeutic agents for use in the disclosed methods may include, but are not limited to, a compound having a formula selected from the group consisting of:

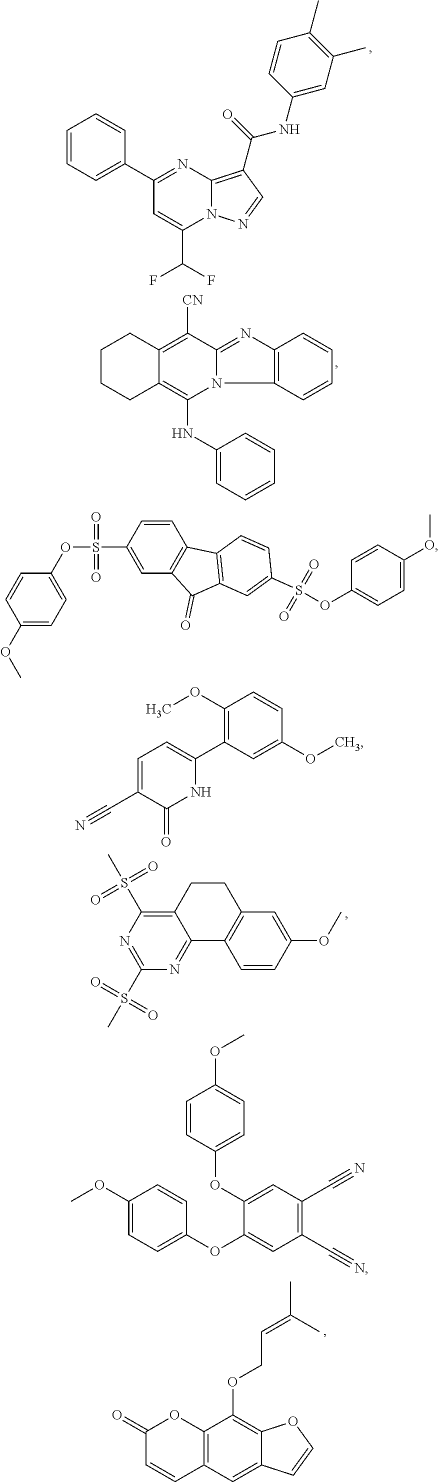

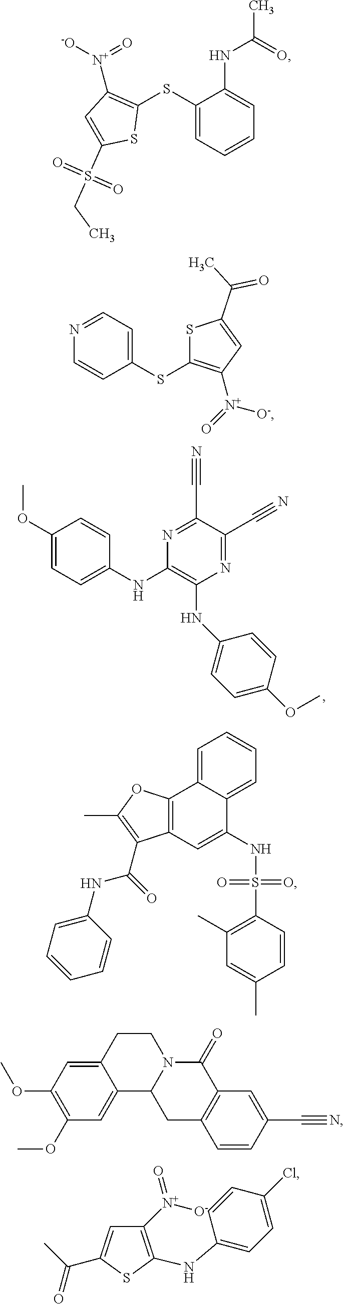

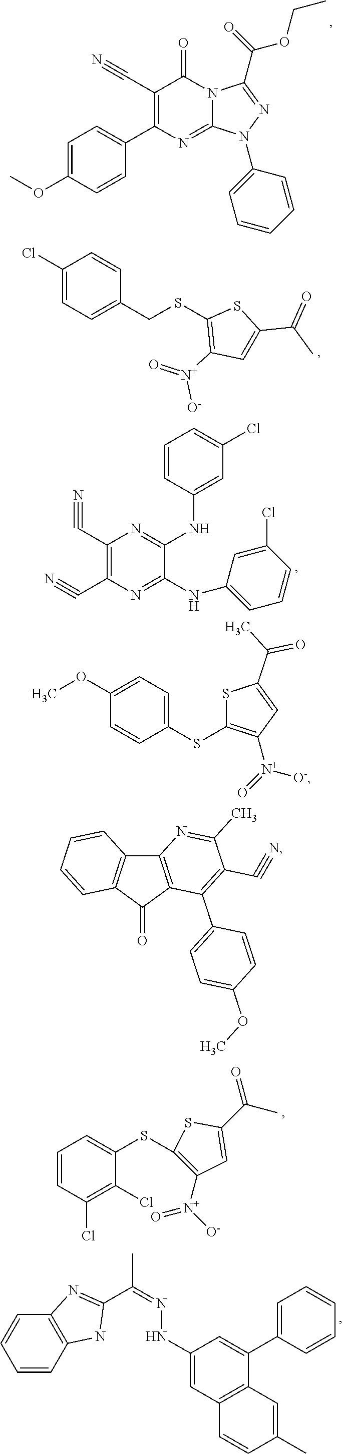

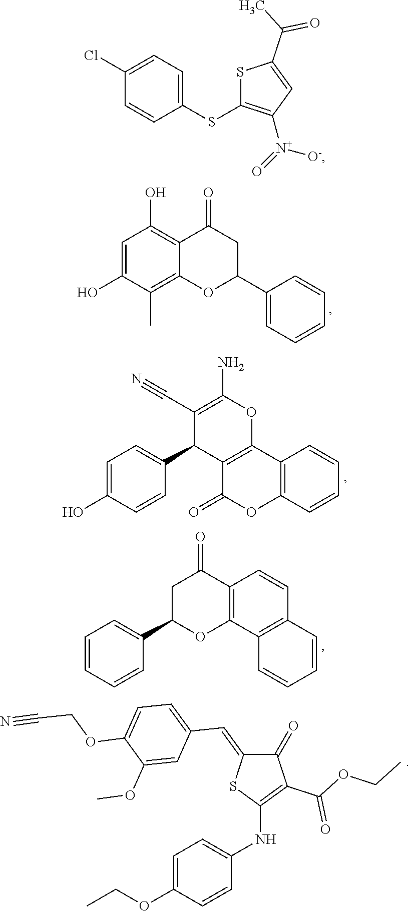

In some embodiments of the disclosed methods, the subject is administered a compound selected from the group consisting of:
7-(difluoromethyl)-N-(3,4-dimethylphenyl)-5-phenylpyrazolo[1,5-a]pyrimidine3-carboxamide,
11-Anilino-7,8,9,10-tetrahydrobenzimidazo[1,2-b]isoquinoline-6-carbonitrile,
2,7-bis(4-methoxyphenyl) 9-oxo9H-fluorene-2,7-disulfonate,
6-(2,5-dimethoxyphenyl)-2-oxo-1,2-dihydropyridine-3-carbonitrile,
2,4-dimethanesulfonyl-8-methoxy5H,6H-benzo[h]quinazoline,
4,5-bis(4-methoxyphenoxy)benzene-1,2-dicarbonitrile,
9-[(3-methylbut-2-en-1-yl)oxy]-7Hfuro[3,2-g]chromen-7-one,
N-(2-{[5-(ethanesulfonyl)-3-nitrothiophen-2-yl]sulfanyl}phenyl)acetamide,
1-[4-nitro-5-(pyridin-4-ylsulfanyl)thiophen-2-yl]ethan-1-one,
bis[(4-methoxyphenyl)amino]pyrazine2,3-dicarbonitrile,
5-{[(2,4-dimethylphenyl)sulfonyl]amino}-2-methyl-N-phenylnaphtho[1,2-b]furan-3-carboxamide,
8-Oxotetrahy dropalmatine,
1-{5-[(4-chlorophenyl)amino]-4-nitrothiophen-2-yl}ethan-1-one,
ethyl 6-cyano-7-(4-methoxyphenyl)-5-oxo-1-phenyl-1,5-dihydro[1,2,4]triazolo[4,3-a]pyrimidine-3-carboxylate,
1-(5-{[(4-chlorophenyl)methyl]sulfanyl}-4-nitrothiophen-2-yl)ethan-1-one,
bis[(3-chlorophenyl)amino]pyrazine-2,3-dicarbonitrile,
1-{5-[(4-methoxyphenyl)sulfanyl]-4-nitrothiophen-2-yl}ethan-1-one,
4-(4-methoxyphenyl)-2-methyl-5-oxo-5H-indeno[1,2-b]pyridine-3-carbonitrile,
1-{5-[(2,3-dichlorophenyl)sulfanyl]-4-nitrothiophen-2-yl}ethan-1-one,
1-(1H-benzimidazol-2-yl)ethanone (6-methyl-4-phenyl-2-quinazolinyl) hydrazone,
1-{5-[(4-chlorophenyl)sulfanyl]-4-nitrothiophen-2-yl}ethan-1-one, Cryptochrysin,
2-amino-4-(4-hydroxyphenyl)-5-oxo-4H,5H-pyrano[3,2-c]chromene-3-carbonitrile,
alpha-naphthoflavanone, and
ethyl 2-(4-ethoxyanilino)-5-[3-methoxy-4-(2-propynyloxy) benzylidene]-4-oxo-4,5-dihydro-3-thiophenecarboxylate.

In some embodiments of the disclosed methods, the therapeutic agent administered to the subject may be the compound having the formula:

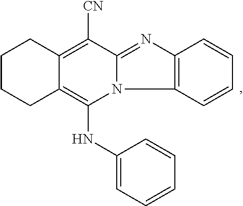 [SMILES: N#Cc1c2c(c(Nc3ccccc3)n3c1nc1ccccc13)CCCC2]

otherwise referred to as 11-Anilino-7,8,9,10-tetrahydrobenzimidazo[1,2-b]isoquinoline-6-carbonitrile.

The disclosed methods also may be performed in order to suppress T_{reg} cell activity in a subject in need thereof. For example, in the disclosed methods the subject may be administered an effective amount of a therapeutic agent that inhibits the activity of USP22, thereby suppressing T_{reg} cell activity in the subject.

In some embodiments, the disclosed methods may also be performed in order to augment the immune response of the subject to an infectious disease in a subject in need thereof.

In some embodiments, the disclosed methods are used to augment the immune response to sudden acute respiratory syndrome coronavirus 2 (SARS-CoV2) infection in a subject in need thereof.

In some embodiments, the disclosed methods are used to augment the immune response of the subject to an infectious disease, in a subject in need thereof. In some embodiments, the therapeutic agent inhibits ubiquitin specific peptidase activity (E.C.: 3.4.19.12) of USP22.

In some embodiments, the disclosed methods of augmenting a subject's immune response to an infectious disease. For example, the therapeutic agent administered to a subject in a need thereof may be a compound having a formula selected from any of the compounds described herein.

Also disclosed are pharmaceutical compositions. In some embodiments, the disclosed pharmaceutical compositions comprise an effective amount of a therapeutic agent having a formula chosen from any of the compounds described herein and a suitable pharmaceutical carrier.

In some embodiments of the disclosed pharmaceutical compositions, the pharmaceutical compositions may comprise an effective amount of a compound is selected from any of the compounds described herein and a suitable pharmaceutical carrier.

In some embodiments, the disclosed pharmaceutical composition may comprise an effective amount of 11-Anilino-7,8,9,10-tetrahydrobenzimidazo[1,2-b]isoquinoline-6-carbonitrile and a suitable pharmaceutical carrier.

In some embodiments, the disclosed pharmaceutical compositions comprise an effective amount of a therapeutic agent that inhibits the biological activity of ubiquitin specific peptidase 22 (USP22).

In some embodiments, the disclosed pharmaceutical compositions comprise an effective amount of the compound for suppressing T_{reg} cell activity.

In some embodiments, the disclosed pharmaceutical compositions comprise an effective amount of the compound for inhibiting ubiquitin specific peptidase activity (E.C. 3.4.19.12) of USP22.

In some embodiments, the disclosed pharmaceutical compositions comprise an effective amount of the compound for inhibiting the biological activity of USP22 when administered to a subject in need thereof.

In some embodiments, the disclosed pharmaceutical compositions comprise an effective amount of the compound for suppressing T_{reg} cell activity when administered to a subject in need thereof.

In some embodiments, the disclosed pharmaceutical compositions comprise an effective amount of the compound for inhibiting ubiquitin specific peptidase activity (E.C. 3.4.19.12) of USP22 when administered to a subject in need thereof.

### Examples

The following Examples are illustrative and should not be interpreted to limit the scope of the claimed subject matter.

### Example 1: Identification of a Deubiquitination Module Essential for T_{reg} Fitness in the Tumor Microenvironment

The highly immunosuppressive tumor microenvironment (TME) favors T regulatory (T_{reg}) cell stability and function, while diminishing the anti-tumor activity of effector T cells. Here, we characterized previously unknown TME-specific cellular and molecular mechanisms that promote intratumoral T_{reg} adaptation. We uncovered the critical role of FOXP3 deubiquitinases, ubiquitin specific peptidase 22 (Usp22) and 21 (Usp21) in T_{reg} stabilization under TME. Specifically, TME stressors including elevated TGF-β, hypoxia, and nutrient deprivation upregulate Usp22 and Usp21 to maintain optimal Foxp3 expression in response to alterations in HIF, AMPK and mTOR activity. The simultaneous loss of both USPs synergizes to alter T_{reg} metabolic signatures and impair suppressive mechanisms, resulting in enhanced anti-tumor activity. Finally, we developed the first Usp22-specific small molecule inhibitor, which significantly reduced intratumoral T_{reg} cells and consequently enhanced anti-tumor immunity. Our findings unveil new mechanisms underlying the functional uniqueness of intratumoral T_{reg} cells and identify Usp22 as an antitumor therapeutic target that inhibits T_{reg} adaptability in the TME.

Tumors have long been recognized as having distinctive properties of growth, invasion, and metastasis, but their ability to evade immune recognition and destruction has recently attracted attention. While neoplastic cells have sufficient antigenicity to promote an anti-tumor immune response, tumors evade the immune system through a variety of mechanisms including the production of immune suppressive mediators and cytokines, defective antigen presentation, and recruitment of immune regulatory cells such as T regulatory (T_{reg}) cells (1, 2). Furthermore, the disorganized vascular system and enhanced rate of proliferation observed in tumors creates a hostile microenvironment depleted of oxygen, glucose, and amino acids while enriched with cytokines and lactic acid (3). Many, if not all, of these alterations in the tumor microenvironment (TME) are known to inhibit anti-tumor immune responses through a variety of mechanisms. Particularly, these TME-derived pressures favorably alter intratumoral (it)T_{reg} cells, resulting in heightened survival and suppressive abilities, while diminishing the anti-tumor effects of effector T (Teff) cells (4-7). Moreover, itT_{reg} cells themselves are known to aid in metastasis, and their increased number correlates with poor clinical outcomes (1,6).

The exact composition of itT_{reg} cells, and whether the majority of this population consists of natural (n)T_{reg} or tumor-induced T_{reg} cells, remains unknown and may differ between tumor types (8). However, it is likely that both populations, although epigenetically distinct, thrive in the TME and further aid in dampening anti-tumor immunity. Interestingly, itT_{reg} cells display upregulated expression of the lineage-defining T_{reg} transcription factor, Forkhead Box P3 (FOXP3) (9, 10), which functions to enhance T_{reg} fitness by augmenting T_{reg} cell stability and suppressive molecular function. Importantly, Foxp3 expression is essential for proper T_{reg} development and function (11). However, the molecular mechanisms underlying how and which TME factors upregulate Foxp3 expression to potentiate itT_{reg} suppressive function remain unknown.

The presence of itT_{reg} cells plays a pivotal role in inhibiting anti-tumor immunity, and is a major hurdle for current tumor-targeting immunotherapies. As T_{reg} depletion through a T_{reg}-specific marker remains challenging (12, 13), the particular pathways that enhance T_{reg} suppressive capabilities within the TME are attractive candidates for new therapeutic targets to diminish itT_{reg} suppressive function. Although Foxp3 is uniquely important for T_{reg} identify and function, it is an intracellular protein whose targeting would require great care as complete inhibition would likely drive significant autoimmunity (11). In addition, specifically targeting a transcription factor like FOXP3 remains technically challenging. Therefore, superior therapeutic candidates will be those that control the expression and stability of Foxp3 specifically in the TME.

Foxp3 expression and stability can be regulated from the transcriptional to the post-translational level, with each layer independently controlling the stability and overall function of T_{reg} cells. Particularly, a newly appreciated layer of Foxp3 regulation and T_{reg} functional modulation is through ubiquitination (14, 15). Ubiquitination of histories on the Foxp3 promoter and conserved non-coding DNA sequence (CNS) regions via E3 ubiquitin ligases results in chromatin condensation and lack of Foxp3 transcription (16). Furthermore, direct ubiquitination of the FOXP3 protein can result in proteasomal degradation. Importantly, ubiquitin may be removed from these sites by deubiquitinating enzymes (DUBs), functioning to both open the chromatin at the transcriptional level, and to stabilize FOXP3 at the protein level (14). The balance between E3 Ligases and DUBs on Foxp3 expression results in an equilibrium state that regulates Foxp3 levels within T_{reg} cells. We and others have discovered three members of the ubiquitin specific peptidase (USP) family as direct modulators of FOXP3 deubiqutination at the transcriptional and/or post-translational level: Usp7, Usp21, and Usp22 (14,17, 18). However, the broad environmental cues and cellular regulation of these deubiquitinases remain unknown. Here, we investigate the role of the TME on the USP-FOXP3 axis, and develop the first Usp22-specific inhibitor capable of antitumor activity. Our study has identified specific TME factors selectively induce FOXP3 deubiquitinases Usp22 and Usp21, but not Usp7 expression to control T_{reg} stability and adaptation.

### Results

Selective Upregulation of FoxP3 Deubiquitinases in itT_{reg} Cells

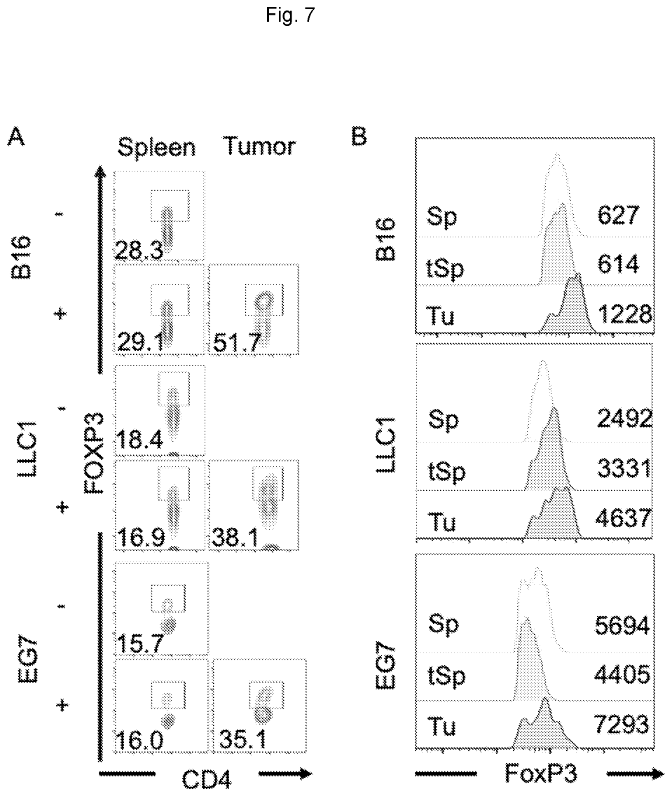

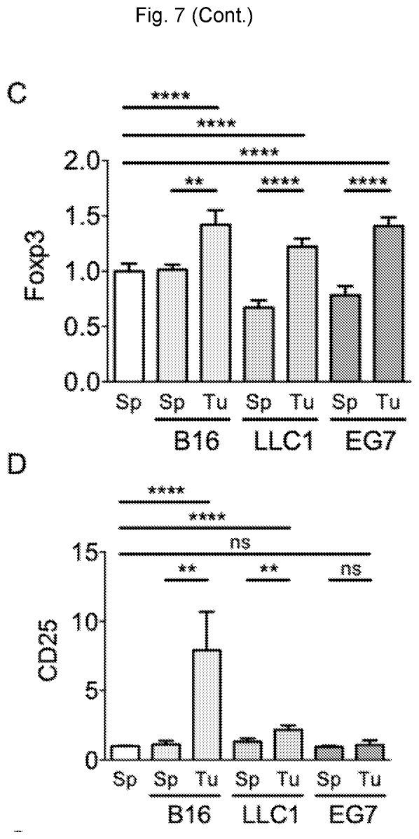

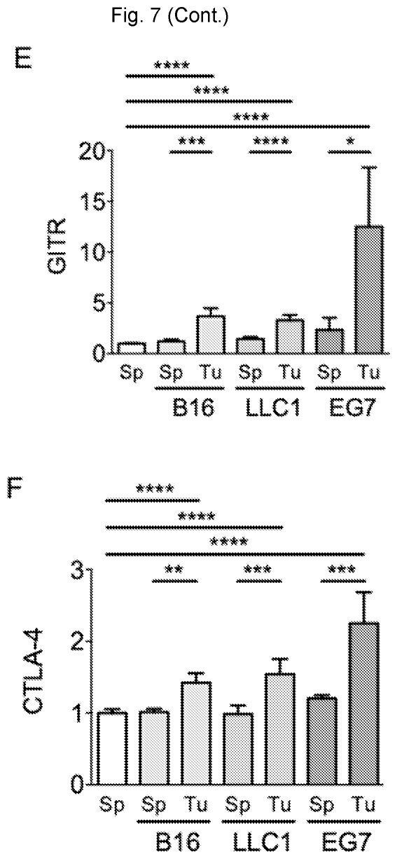

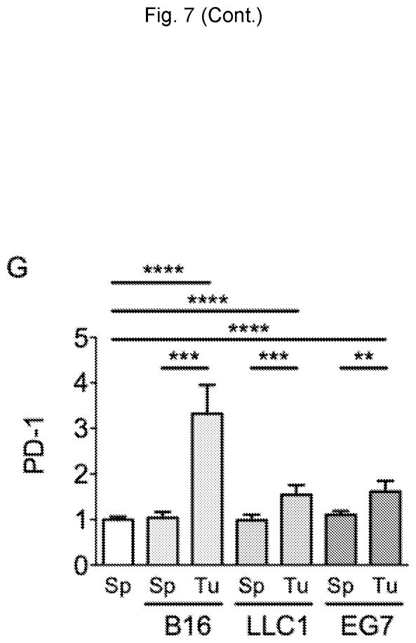

Since tumors create a hostile microenvironment where immune cell function is greatly altered, we began by characterizing the suppressive profiles of murine itT_{reg} cells (FIG. 7). Upon subcutaneous injection of B16 melanoma, LLC1 Lewis Lung Carcinoma, and EG7 Lymphoma into WT Foxp3^{YFP-cre} (WT) mice, itT_{reg} displayed both increased percentages of FOXP3+ cells and FOXP3 protein levels relative to splenic T_{reg} cells within the same mouse as well as against a non-challenged control mouse (FIG. 7A-C). Furthermore, itT_{reg} cells in each tumor type exhibited increased surface expression of multiple known T_{reg} suppressive markers including CTLA-4 and PD-1 (FIG. S1D-G). These data suggest that itT_{reg} cells have elevated immune suppressive functions through the upregulation of FOXP3 and surface inhibitory receptors, which is consistent with previous studies demonstrating that human intratumor T_{reg} cells display enhanced suppressive function (4).

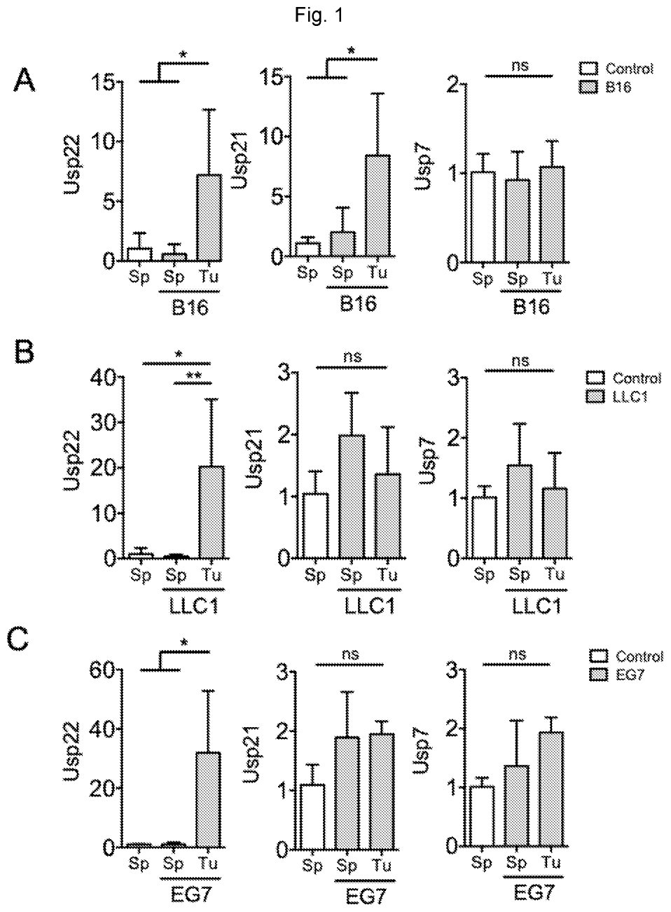

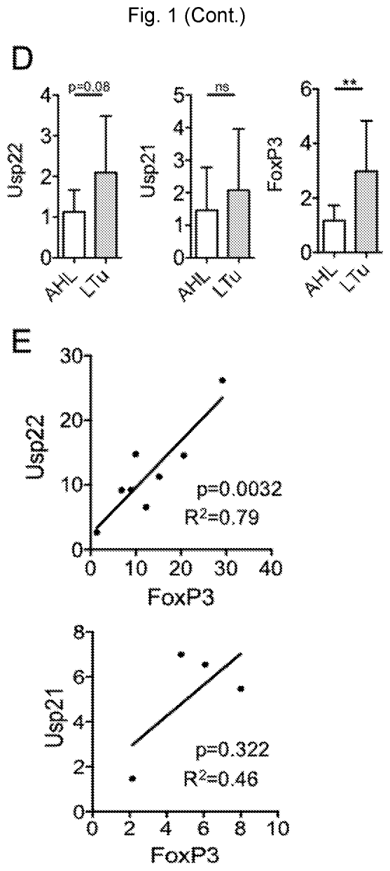

As the three FOXP3-targeting USPs aid in maintaining FOXP3 stability (16-18), we hypothesized that modulation of their expression may drive the FOXP3 upregulation in itT_{reg} cells. Interestingly, the mRNA level of Usp22 was consistently increased within itT_{reg} cells in comparison to the peripheral T_{reg} cells harvested from same mouse or non-challenged controls, but Usp7 mRNA level was unchanged. In contrast, Usp21 mRNA level was only increased under B16 challenge, suggesting that Usp21 upregulation in T_{reg} cells occurs only under certain TME conditions (FIG. 1A-C). These data indicate that one or many factors in the TME upregulate both Usp22 and Usp21 transcription to potentially stabilize FOXP3, leading to stabilized itT_{reg} function. To support this notion, we further confirmed that Usp22 is upregulated in T_{reg} cells isolated from human tumor lung tissue patient samples (LTu) in comparison to adjacent healthy lung tissue (AHL) (FIG. 1D). This upregulation shows a strong positive correlation with FOXP3 upregulation within the LTu patient samples, suggesting Usp22 promotes Foxp3 expression in itT_{reg} cells in human tumors (FIG. 1D-E). Similar to our observation from the syngeneic lung cancer model, Usp21 was not increased in human lung tumor itT_{reg} cells nor did it have a significantly positive correlation with Foxp3 (FIGS. 1D and F), suggesting that Usp22 is the more dominant USP in T_{reg} cells within the tumor at least in lung cancer.

### Tumor-Derived TGF-β Selectively Induces Usp22 and Usp21 in T_{reg} Cells

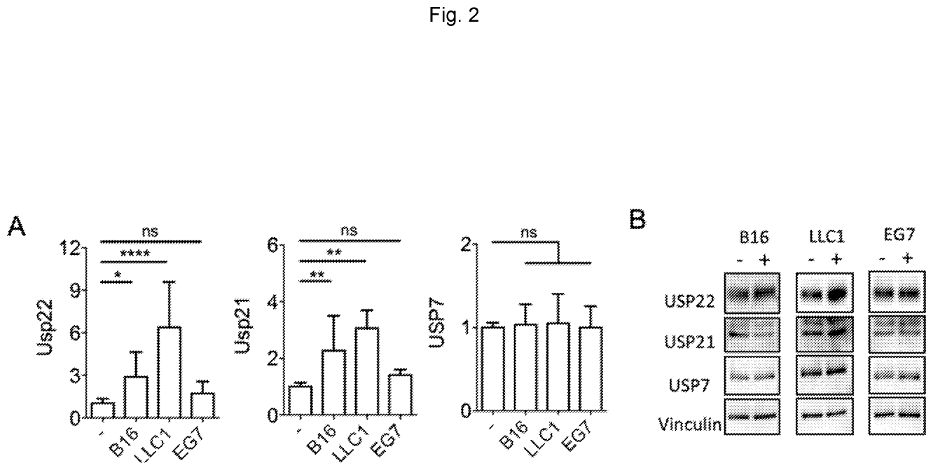

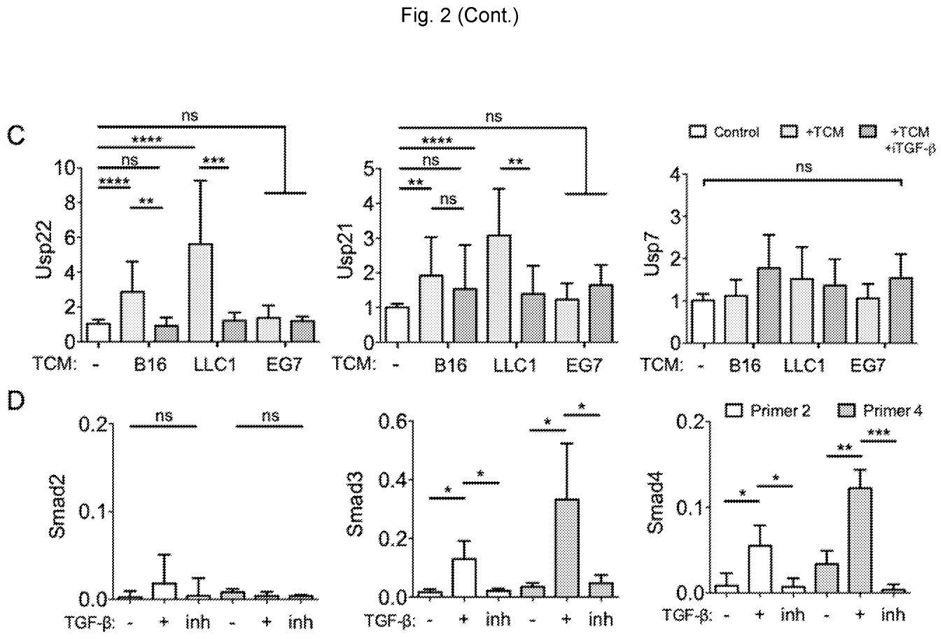

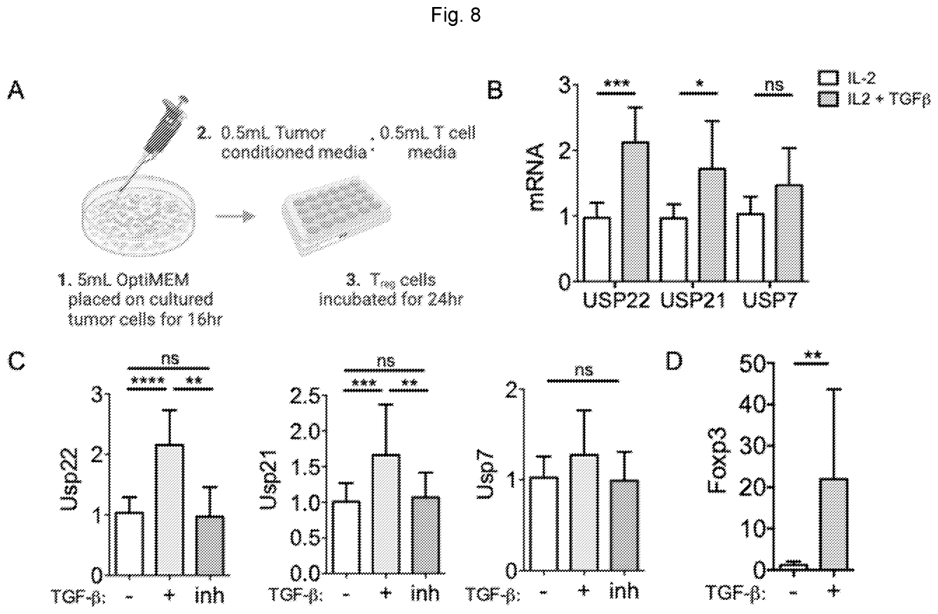

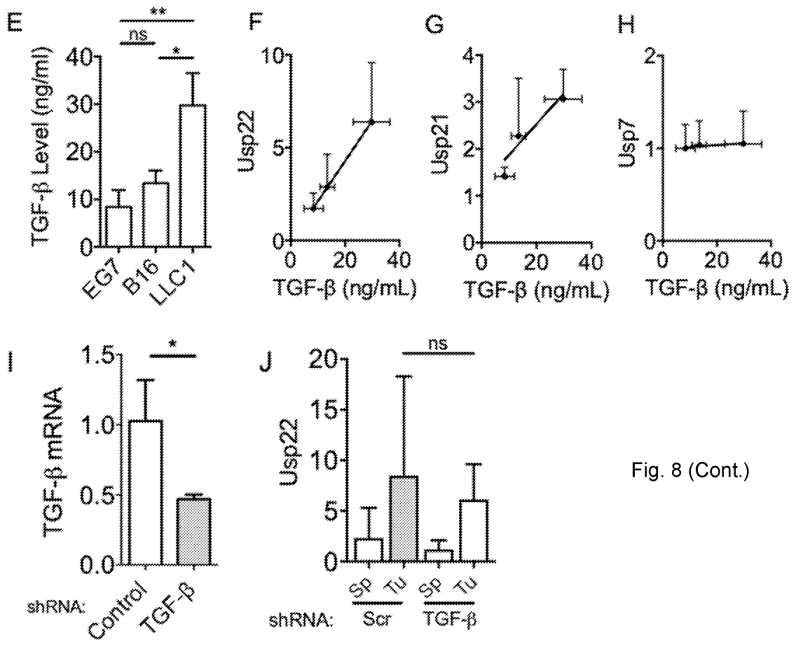

As soluble factors secreted by the tumors are known to alter immune cell function (19, 20), we investigated the role of TME-soluble factors in regulating Usp22 and Usp21 in itT_{reg} cells. We exposed in vitro induced (i)T_{reg} cells to media obtained from cultured tumor cells (tumor conditioned media or TCM) (FIG. 8A). Interestingly, TCM from B 16 and LLC 1, but not EG7 cells, enhanced Usp22 and Usp21 mRNA levels (FIG. 2A). In contrast, the levels of Usp7 remained unchanged, recapitulating the results in FIG. 1. Similar to the mRNA levels, USP22 and USP21 protein levels were increased upon incubation with LLC1 TCM (FIG. 2B). Consistently, the addition of EG7 cultured media was not able to enhance any of the USPs at the protein level (FIG. 2B), indicating that specific tumor types selectively inducing Foxp3 deubiquitinases in T_{reg} cells.

Many types of tumors secrete large amounts of TGF-β, which dampens immune responses and promotes metastasis (21, 22). Together with the fact that TGF-β is particularly important for iT_{reg} generation and stability (23), we speculated that TGF-β could aid in enhancing Foxp3 expression in itT_{reg} cells through induction of Usp22 and Usp21. Indeed, mRNA levels of both Foxp3-targeting USPs were increased when TGF-β was added to the media of iT_{reg} cells, while Usp7 showed no such increase (FIG. 8B). This increase of both Usp22 and Usp21 expression was largely diminished by the addition of a TGF-β inhibitor (LY 3200882) (FIG. 8C). Importantly, the level of Foxp3 mRNA rose concurrently with the levels of Usp22 and Usp21 (FIG. 8D), demonstrating that the TGF-β can further enhance FoxP3 expression through Usp22 and Usp21 induction.

To further determine if TGF-β is implicated in TCM-driven Usp22 and Usp21 upregulation, we added the TGF-β inhibitor to the TCM from each of the aforementioned tumor cell lines. Indeed, the TGF-β inhibitor completely diminished the mRNA enhancement of Usp22 (FIG. 2C), signifying that TGF-β is the primary factor in the B16 and LLC1 TCM that enhances Usp22 expression. Interestingly, the Usp21 level was also diminished when the inhibitor was added to the LLC1 TCM, but was not under B16 TCM condition (FIG. 2C). It is possible that this difference could be due to the quantity of TGF-β secreted by the tumor cell lines into the medias. Indeed, LLC1 cells secreted significantly higher amounts of TGF-β than both B16 and EG7 cells (FIG. 2SE), which positively correlates with observed increase in Usp22 and Usp21 mRNA expression (FIGS. 8F and G).

The levels of Usp7 remain unchanged under all treatment groups and displayed no correlation to the increasing level of TGF-β in the various tumor types (FIG. 2C and FIGS. 8B-C and H). Therefore, our data identify TGF-β as a critical soluble factor to selectively induce Usp22 and Usp21 in T_{reg} cells.

To determine if tumor derived TGF-β is important in upregulating Usp22 and Usp21 within the TME proper, we determined whether the levels of Usp22 and Usp21 in itT_{reg} cells infiltrating B16 melanomas lacking TGF-β are still upregulated. shRNA knockdown largely diminished the TGF-β expression in B16 cells (FIG. 8I). However, Usp22 levels, while with a trend of reduction in itTregs from B 16 melanoma lacking TGF-β comparing to those in tumors treated with scramble shRNA, did not achieve any statistical significance, indicating that although tumor derived TGF-β is sufficient to induce Usp22 and Usp21 both in vitro, other TME factors are also at play which overcome the effect of TGF-β knockdown at the current experimental setting.

### TGF-β Signaling Upregulates Usp22 and Usp21 Through Distinctive Pathways

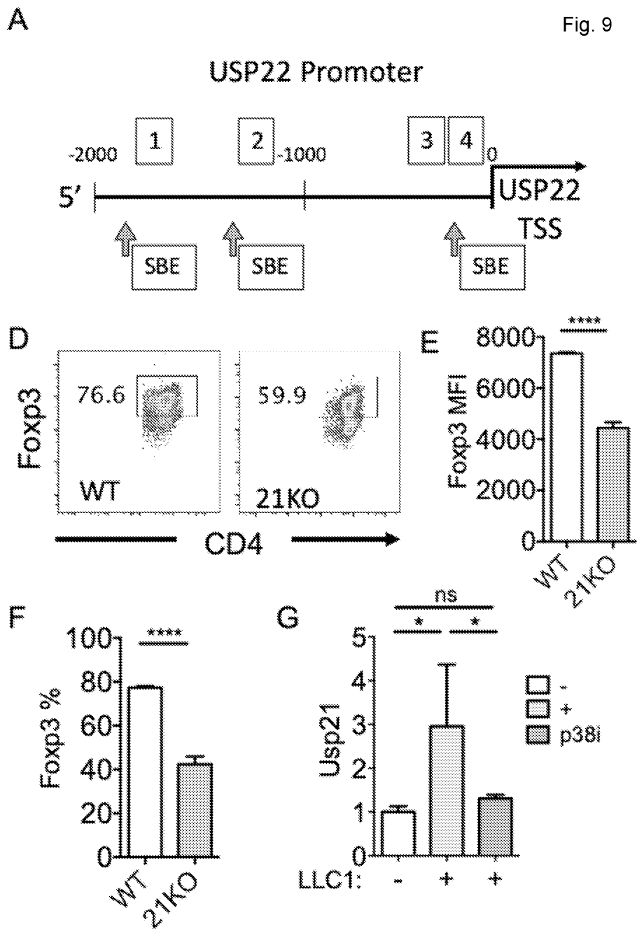

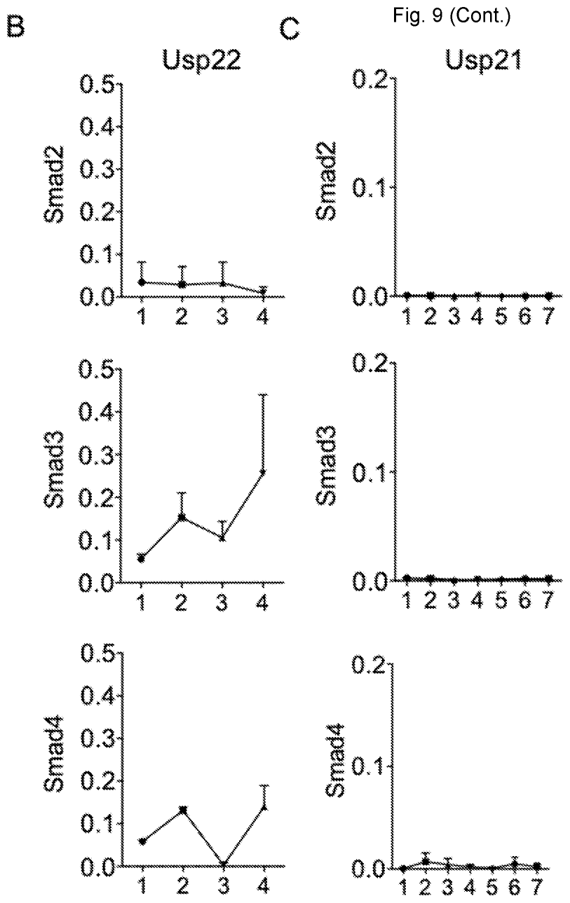

To uncover the mechanism by which TGF-β acts on Usp22 and Usp21 transcription, we first investigated the canonical TGF-β signaling pathway, which works through the co-activating SMAD transcription factors (homologues of the Drosophila protein, mothers against decapentaplegic (Mad) and the Caenorhabditis elegans protein Sma) including SMAD2, SMAD3 and SMAD4 through specifically binding to the SMAD-binding element (SBE) (24, 25). We scanned along the promoter regions of both Usp22 and Usp21 for sequences of conserved SBE. Along the Usp22 promoter, we found three promising regions for which we made primers and assessed the SMAD binding capacity (FIGS. 9A and B). Chromatin immunoprecipitation (ChIP) analysis detected that SMAD3 and SMAD4, but not SMAD2, bind to Usp22 promoter at around 300 and 1200 base pairs upstream of the transcription start site (FIG. 9B). SMAD binding at both sites was ablated upon the addition of the TGF-β inhibitor, demonstrating that SMAD3 and SMAD4 binding to the Usp22 promoter is due directly to TGF-β signaling (FIG. 2D). SMAD2 showed no binding capacity to any regions of the Usp22 promoter (FIG. 2D; FIG. 9B); likely due to steric hinderance blocking its direct DNA interaction (26).

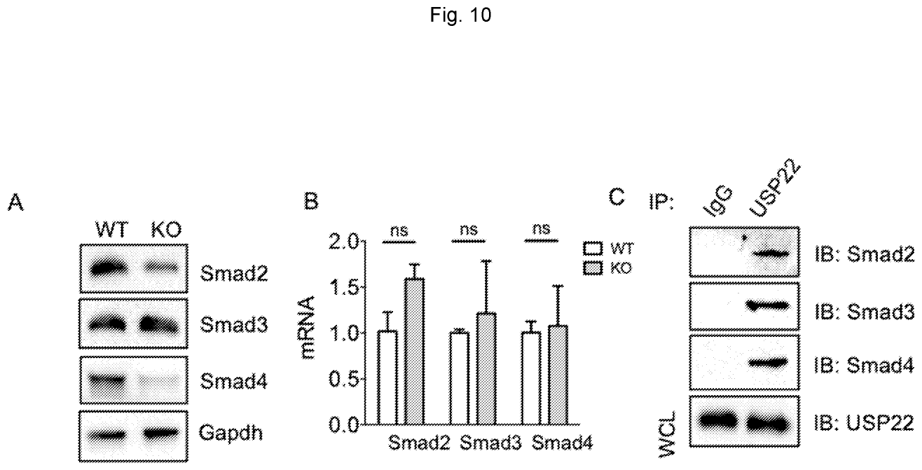

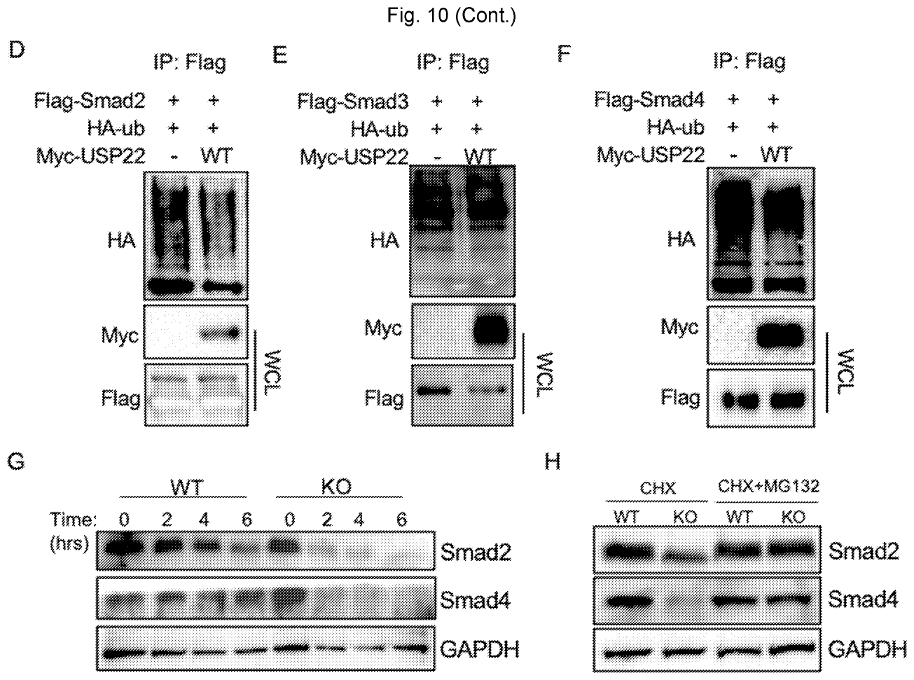

We have recently observed that, although Usp22-null iT_{reg} cells polarize normally with high levels of TGF-β, sub-optimal polarization conditions resulted in a significant decrease in FOXP3 MFI and percentage relative to the WT iT_{reg} cells (16). This suggests an important function of Usp22 in perpetuating TGF-β signaling within iT_{reg} polarization. Indeed, Usp22-null iT_{reg} cells display a significant deficiency in both SMAD2 and SMAD4 protein levels compared to WT iT_{reg} cells, with no difference in their mRNA levels (FIGS. 10A and B), suggests Usp22 functions as a positive regulator for the TGF-β signaling pathway through stabilizing SMADs at the protein level. This decrease is possibly due to enhanced SMAD ubiquitination and proteasomal degradation upon Usp22 deletion, as Usp22 is a DUB. Indeed, Usp22 interacts with and deubiquitinates both SMAD2 and SMAD4 (FIGS. 10C-D and F). Although Usp22 interacts with SMAD3, it does not act as a DUB of SMAD3 (FIGS. 10C and E), suggesting it acts specifically through stabilizing SMAD2 and SMAD4. Particularly, USP22-null iT_{reg} cells displayed enhanced degradation of SMAD2 and SMAD4 upon cycloheximide treatment relative to WT, which is rescued upon proteasomal inhibition with MG132 treatment (FIGS. 10G and H). Therefore, our data suggests that USP22 functions to reciprocally enhance TGF-β signaling through SMAD2 and SMAD4 protein stabilization. This act ensures upregulation of itself through a positive feedback loop, further ensuring Foxp3 expression in itT_{reg} cells.

Unlike with Usp22, no SBEs were found when scanning the Usp21 promoter, implying that TGF-β induces Usp21 expression independent of SMADs. Indeed, none of the regions showed binding capacity of any of the tested SMAD proteins, confirming that Usp21 expression is not induced through canonical TGF-β signaling (FIG. 9C). However, lack of Usp21 in iT_{reg} cells results in diminished Foxp3 expression in vitro, leaving the opportunity for non-canonical TGF-β signaling to drive Usp21 induction and result in stabilization of Foxp3 (FIG. 9D-F). Indeed, inhibition of p38, a Smad-independent TGF-β mediated MAP kinase, restrained TGF-β-mediated Usp21 induction (FIG. 9G). Altogether, these data indicate that Usp22 and Usp21 are mediated through distinct TGF-β signaling pathways.

Hypoxia Selectively Induces T_{reg} Usp22, which Supports Foxp3 Expression

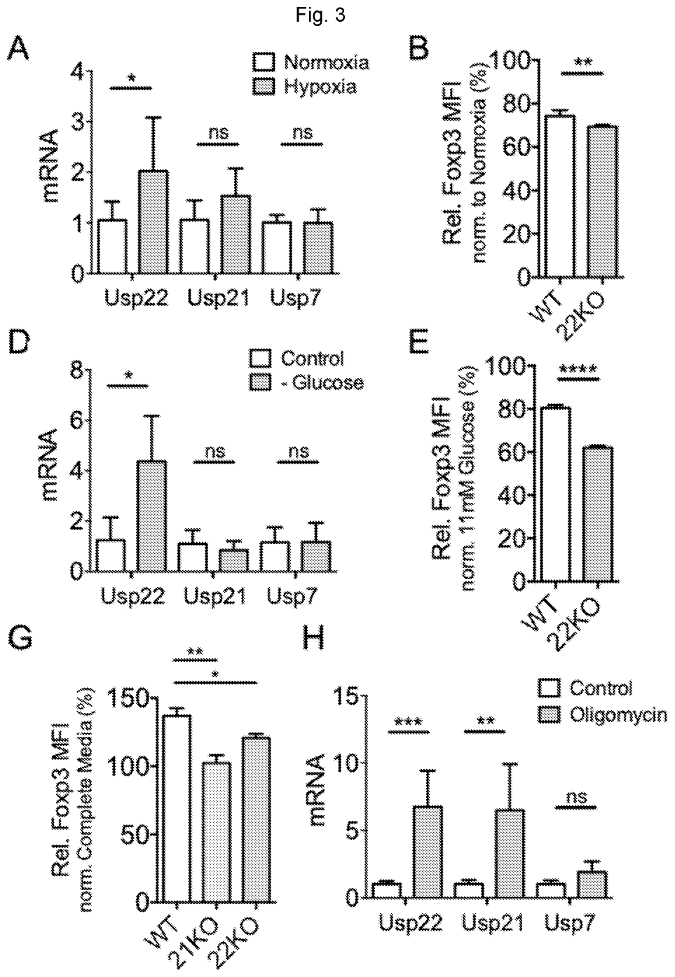

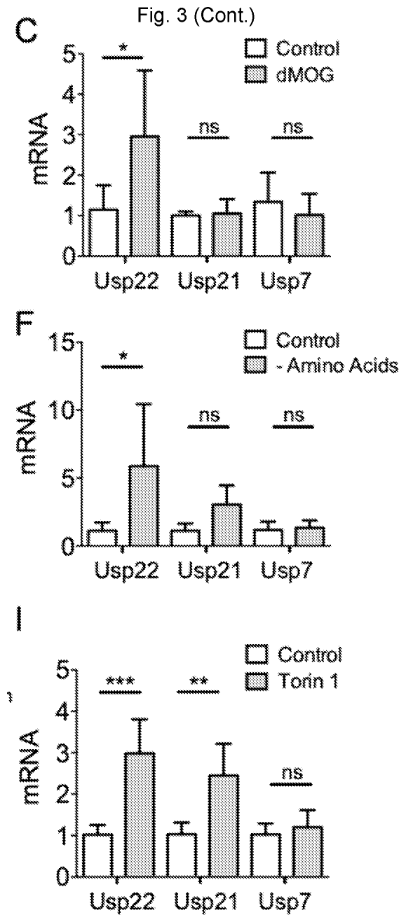

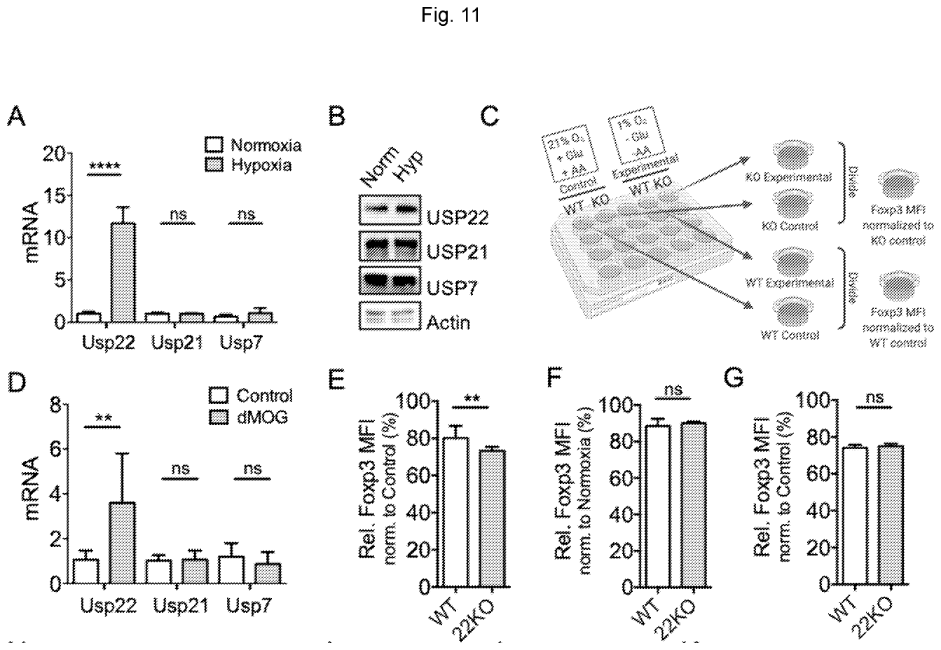

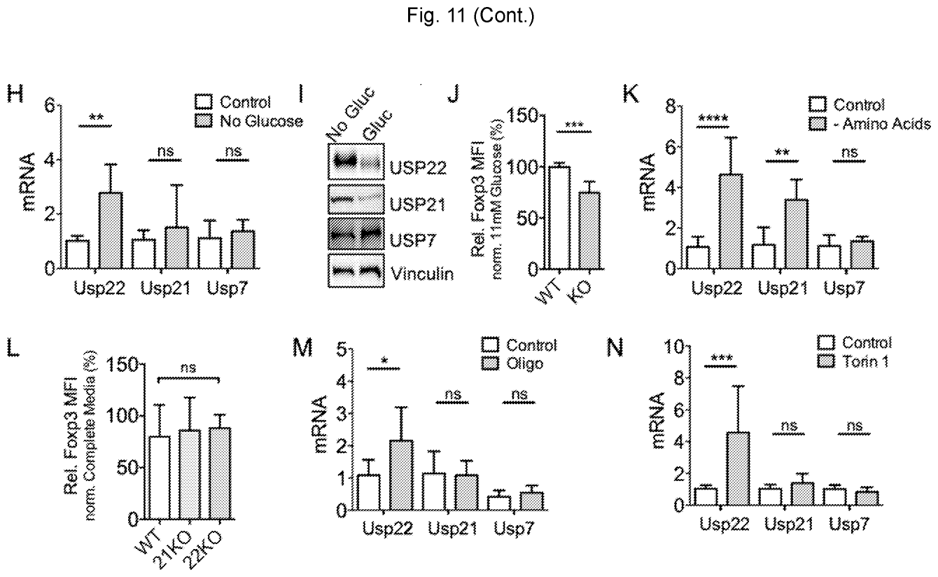

Although tumor derived TGF-β was central to upregulating T_{reg} Usp22 and Usp21 in vitro, TGF-β suppression was insufficient to abolish Usp22 upregulation in itT_{reg} cells (FIG. 8I-K), implying that additional TME factors may influence itT_{reg} stability and function through USPs. In addition to tumor cell secreted factors, tumor-driven hypoxia has been repeatedly implicated in FOXP3 stability and T_{reg} cell function (27, 28). A known negative prognostic factor in solid tumors (3, 29), hypoxia preferentially downregulates T cell proliferation, receptor signal transduction, and effector function while increasing T_{reg} cell suppressive capabilities (27, 30, 31). We, therefore, investigated the effects of hypoxia on USP levels in T_{reg} cells. Surprisingly, only Usp22 expression was enhanced under hypoxic conditions at both the mRNA and protein levels (FIG. 3A and FIGS. 11A and B). Therefore, we speculated that Usp22 could function as a stabilizer of FOXP3 under the hypoxic conditions in the TME and performed a FOXP3 stability assay (FIG. 11C). Indeed, Usp22-deficient nT_{reg} cells show a reduced ability to sustain FOXP3 expression under hypoxic conditions (FIG. 3B), signifying that Usp22 is required for FOXP3 stabilization under the hypoxic conditions found within the TME.

Under hypoxic conditions, hypoxia inducible factors α (HIF-α) are stabilized resulting in the activation of a transcriptional program that promotes cellular adaptation to low oxygen levels (32). HIF-α are known to have two functional binding sites on the Usp22 promoter (33), suggesting that hypoxic induction of Usp22 may be HIF-α-dependent. Indeed, incubation with hypoxia-independent HIF-α activator, dimethyloxalylglycine (dMOG), increased Usp22 mRNA level in both nT_{reg} and iT_{reg} cells (FIG. 3C; FIG. 11D), indicating that hypoxia-induced Usp22 expression is involved in FoxP3 stabilization. To support this, we further showed that Usp22-deficient nT_{reg} cells displayed decreased stability of FOXP3 following treatment with dMOG, confirming that Usp22-dependent FOXP3 stabilization under hypoxic conditions is HIF-α dependent (FIG. 1 IE). In contrast, this FOXP3 stabilization was not observed in iT_{reg} cells under hypoxic conditions or with dMOG treatment (FIGS. 11F and G). This could be due to the lack of TGF-β present in the experimental conditions, which is pivotal for Foxp3 expression stabilization in iT_{reg} cells. Regardless, these results demonstrate the importance of Usp22 in hypoxia-mediated T_{reg} cell FOXP3 expression within the TME.

### Metabolic Alterations in the TME Induce Usp22 and Usp21 to Promote Foxp3 Stability

In addition to oxygen, glucose levels in the TME are often decreased, in part through its enhanced uptake by tumor cells which compete with the glucose necessity of the highly glycolytic Teff cells (34, 35). Conversely, FOXP3 promotes oxidative phosphorylation over glycolysis in T_{reg} cells, potentially giving them a functional advantage within the TME (5, 36, 37). Therefore, we hypothesized the observed T_{reg} cell advantage in nutrient deprived environments could exist partially as a consequence of USPs mediated stabilization of Foxp3 expression. Indeed, Usp22 mRNA and protein levels were increased in T_{reg} cells upon glucose deprivation (FIG. 3D and FIGS. 11H and I). Additionally, Usp22-deficient T_{reg} cells have significantly lower FOXP3 maintenance under glucose deprivation compared to WT T_{reg} cells, demonstrating that Usp22 functions to stabilize FOXP3 under glucose-restricted conditions (FIG. 3E and FIG. 11J).

Along with the competition for glucose, a scarcity of amino acids within tumors may also alter immune cell function (35). Importantly, amino acid starvation is known to enhance T_{reg} cell induction (38). To investigate the role of USPs in amino acid starvation induced Foxp3 expression we cultured T_{reg} cells in media lacking amino acids. Indeed, amino acid starvation led to increased expression of Usp22 and Usp21, but not Usp7, in nT_{reg} and iT_{reg} cells (FIG. 3F; FIG. 11K). Furthermore, the stability of FOXP3 in amino acid starved nT_{reg}, but not iT_{reg}, cells is reduced by the deficiency of Usp22 or Usp21 (FIG. 3G; FIG. 11L). In environments deplete of both glucose and amino acids, activation of adenosine monophosphate-activated protein kinase (AMPK) suppresses anabolic metabolism while upregulating oxidative metabolism to promote cellular survival (39), suggesting AMPK activation is involved in Usp22 or Usp21 upregulation. So, we treated T_{reg} cells with an inhibitor of mitochondrial ATP synthase, Oligomycin A, and measured USP mRNA levels. Indeed, oligomycin treatment increased both Usp22 and Usp21, but not Usp7, mRNA level in nT_{reg} (FIG. 3H), further supporting our observation that glucose deprivation and subsequent energy stress induces Usp22 expression in T_{reg} cells.

It is well known that AMPK functions in balance with mammalian target of rapamycin (mTOR) signaling to regulate the cellular metabolic state (39). Intriguingly, pharmacologic inhibition of mTOR also resulted in increased Usp22 and Usp21, but not Usp7, expression in nT_{reg} cells (FIG. 3I). In iT_{reg} cells, however, Usp21 was not upregulated at the mRNA level upon AMPK activation or mTOR inhibition (FIGS. 11M and N), suggesting cell-type specificity of the response. Collectively, these findings suggest that the global metabolic state as determined by the balance of AMPK and mTOR activity, act to modulate Foxp3 expression and stability through Usp22, and to a lesser extent Usp21, in T_{reg} cells.

It has been proposed that itT_{reg} cells better adapt to the metabolically stressful conditions of the TME, which offers them a functional advantage over Teff cells (5, 19). Combined, our data suggests that alterations in the microenvironment can drive increased levels of Usp22 and Usp21 potentially through modulation of HIFα, AMPK, and mTOR activity to enhance T_{reg} stability in the tumor microenvironment.

### USP22 and USP21 Modulate T_{reg} Fitness Through Distinct Pathways

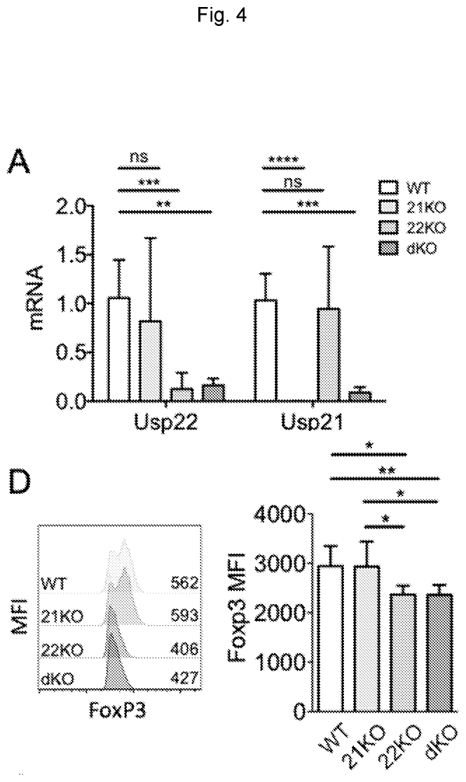

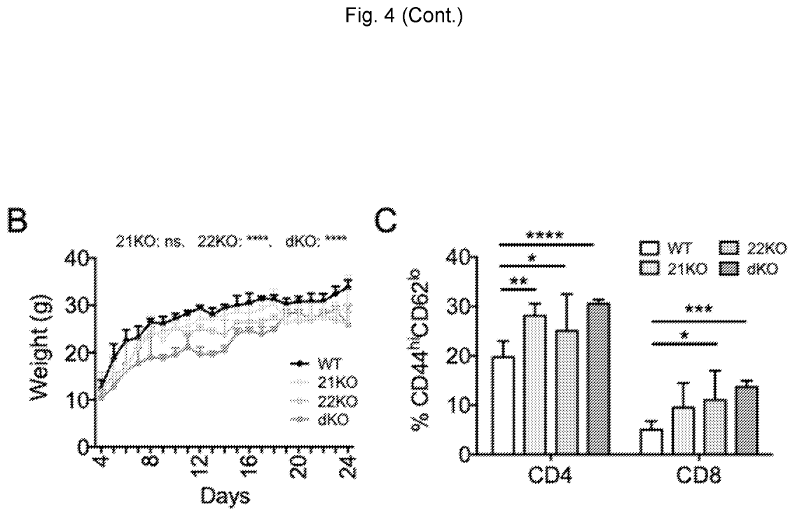

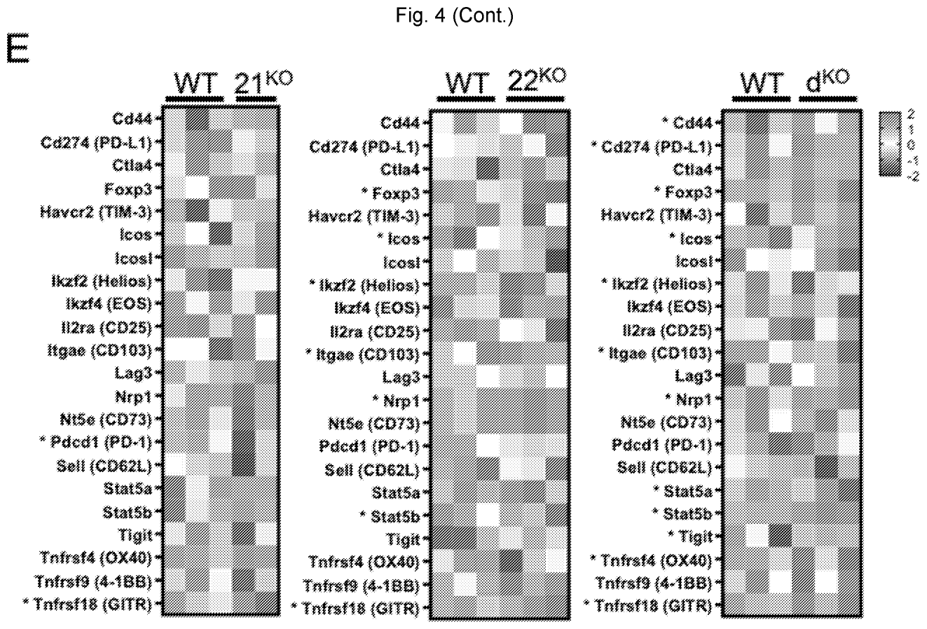

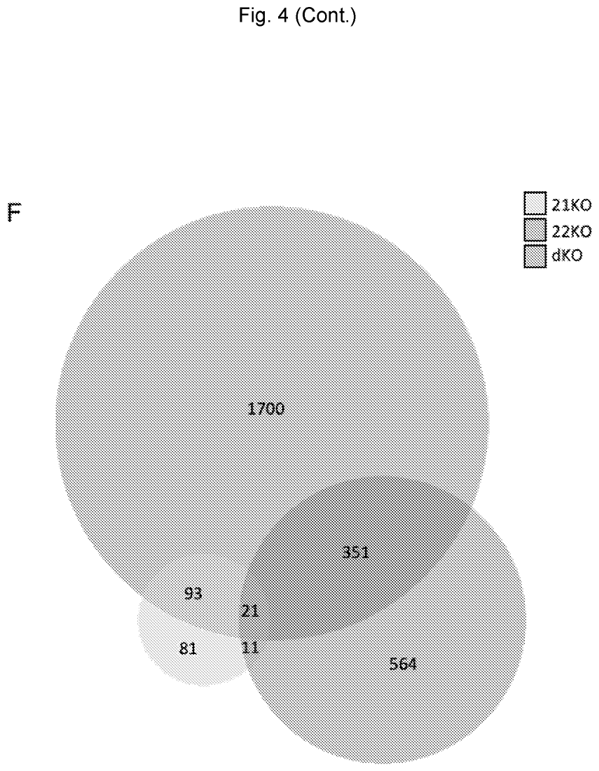

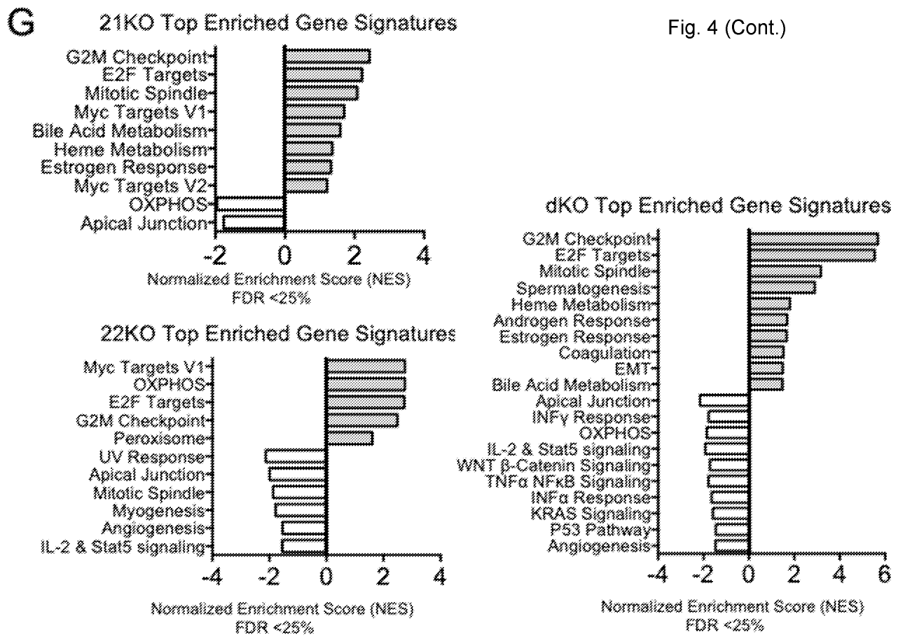

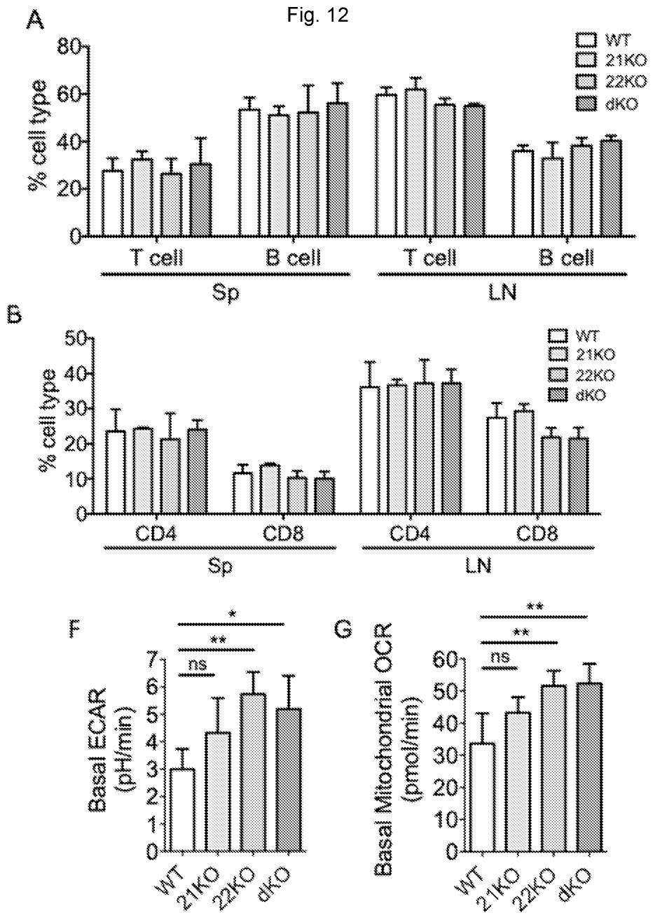

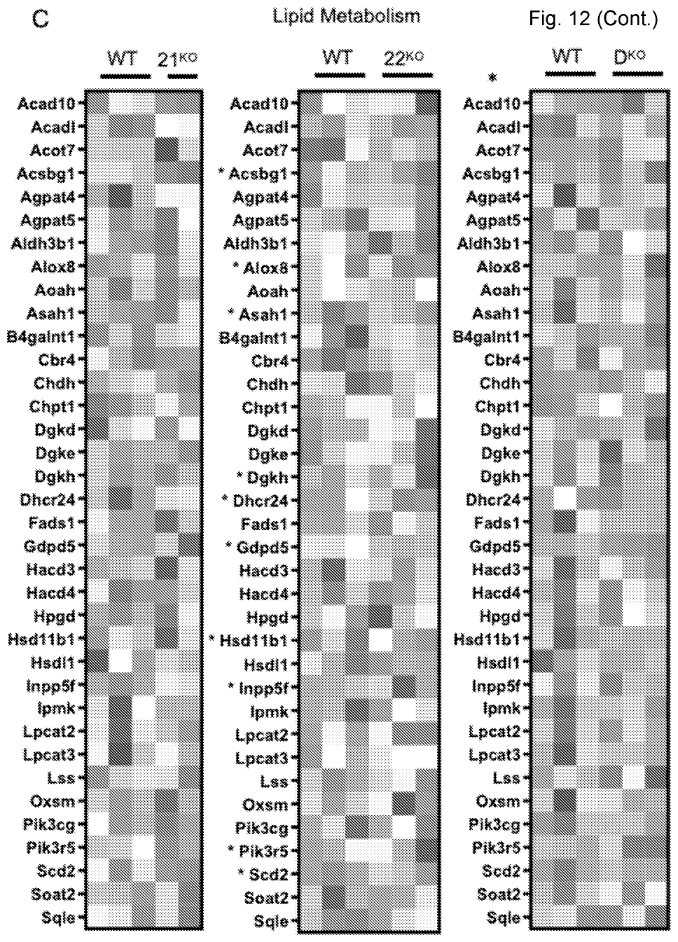

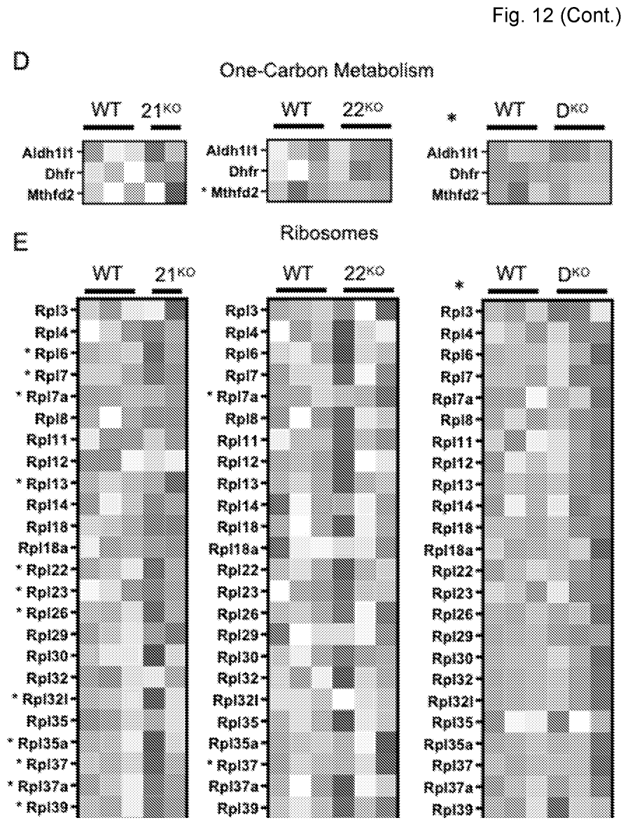

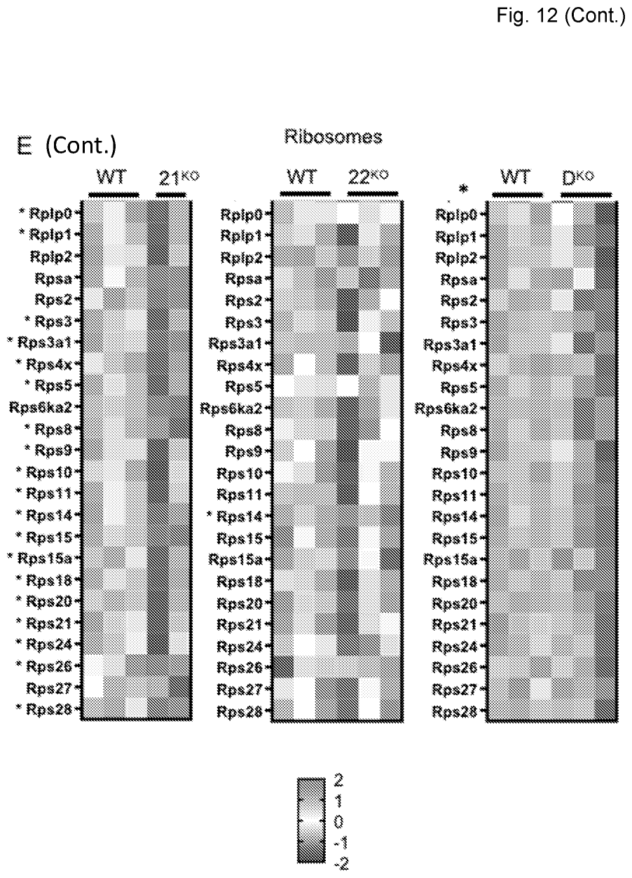

Our discoveries thus far have suggested that Usp22, and to a lesser extent Usp21, are important in maintaining FOXP3 expression and thus T_{reg} fitness in the TME through multiple pathways. To study their combined functionality in vivo, we generated a strain of T_{reg}-specific Usp22 and Usp21 double knockout (dKO) mice by breeding Usp21^{f/f} mice with Usp22^{f/f}FoxP^{3YFP}ere single knockout mice. This breeding strategy gave us the T_{reg}-specific knockout of Usp22 (22KO), Usp21 (21KO), and the dKO, all of which were confirmed via qPCR (FIG. 4A). Deletion of either Usp22, Usp21, or both in T_{reg} cells did not alter the frequency of either B or T cells in the spleens of 6-week-old mice (FIGS. 12A and B). Importantly, while the mice display similar weights early in life, by 24 weeks of age the 22KO and dKO animals are consistently smaller in size compared to WT (FIG. 4B).

Unsurprisingly, all three KO groups showed significant increase in CD44^{hi}CD62^{lo} activated splenic Teff cells in comparison to age matched WT mice, consistent with the development of low level, progressive inflammation with age (FIG. 4C). Importantly, only the 22KO and dKO mice showed decreases in Foxp3 expression and significant reductions in T_{reg} cell-associated suppressive markers (FIGS. 4D and E). Although a previous study reported that the 21KO mice develop age-related impairments in T_{reg} cell function and number secondary to impaired Foxp3 expression (18), our 8-week-old mice showed no changes in Foxp3 expression, suggesting that Usp21-mediated Foxp3 stabilization may be dispensable outside of the TME.

Interestingly, transcriptional profiling revealed more T_{reg} cell suppressive markers were differentially expressed in the dKO mice than in either single KO animal when compared to WT gene expression (FIG. 4E), suggesting a possible synergism between the loss Usp22 and Usp21 on T_{reg} cell stability and function. Furthermore, differentially expressed genes (DEGs) between the 21KO and the 22KO were relatively distinct (FIG. 4F). Although gene set enrichment analysis (GSEA) of both single KO mice showed changes in many cell cycle and proliferative pathways, such as G2M checkpoints and E2F targets, as well as changes in oxidative phosphorylation (FIG. 4G), there were only a total of 32 overlapping differentially expressed genes between the 21KO and the 22KO (FIG. 4F). Importantly, T_{reg} cells from the dKO animals displayed a GSEA and bulk gene expression signature that merged the changes found in each of the single KO mice, suggesting that the loss of both Usp22 and Usp21 synergize to diminish T_{reg} cell function (FIG. 4G).

As we demonstrated that both Usp22 and Usp21 are regulated by metabolic alterations in the TME, it was particularly interesting to identify disruption of multiple metabolic pathways in each of the KO animals. In fact, T_{reg} cells from dKO mice had profound changes in lipid metabolic processes, one carbon metabolism, and ribosomal biogenesis (FIG. 12C-E). Interestingly, Usp22-null T_{reg} cells, but not Usp21 deficient cells, displayed similar alterations in both lipid metabolism and one-carbon metabolism to the dKO T_{reg} cells (FIGS. 12C and D). In contrast, T_{reg} cells from 21KO and dKO mice showed profound decreases in ribosomal gene expression, which was not identified in the Usp22-null T_{reg} cells (FIG. 12E), suggesting distinct pathways by which Usp22 and Usp21 modulate T_{reg} fitness. Our in vitro metabolic flux analysis further demonstrated that, unlike the 21KO, both 22KO and the dKO display enhanced mitochondrial oxygen consumption (OCR) and extracellular acidification rates (ECAR) (FIGS. 12F and G), suggesting that Usp22 may play an essential role in modulating the metabolic state of regulatory T cells.

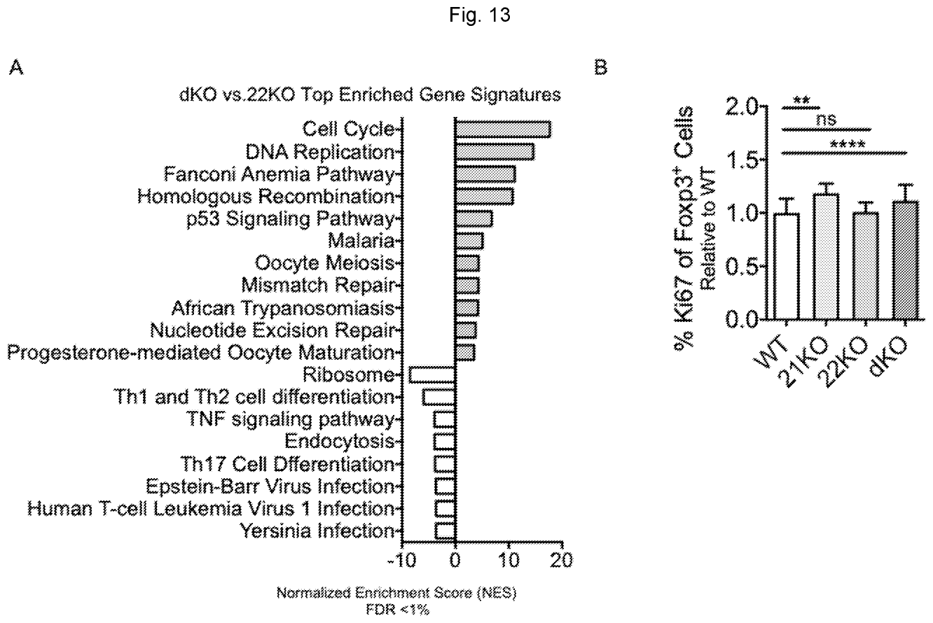

As Usp21 seemed to have a Foxp3-independent role in T_{reg} function, we compared the DEGs from the 22KO and the dKO mice in order to determine the contribution of Usp21 to the dKO phenotype. Interestingly, we noticed significant changes in cell cycle pathways and effector differentiation pathways (FIG. 13A), suggesting a loss of homeostasis in the T_{reg} compartment. Indeed, Ki67 staining revealed an increase in proliferation of the 21KO and dKO T_{reg} cells in comparison to WT T_{reg} cells, but not in the 22KO (FIG. 13B), indicating that the normally highly suppressive effector T_{reg} has an increased proliferative capacity with a downregulation of functional genes.

Collectively, these data imply that both Usp22 and Usp21 modulate T_{reg} cell metabolism although seemingly through unique pathways to maintain T_{reg} stability and function.

### Usp22 and Usp21 Deletion in T_{reg} Cells Synergize to Enhance Anti-Tumor Immunity

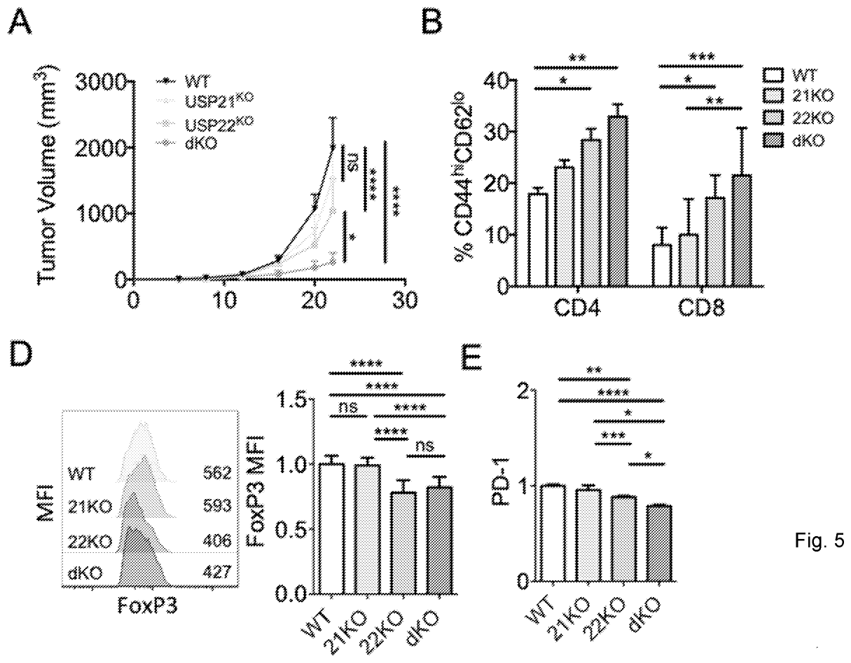

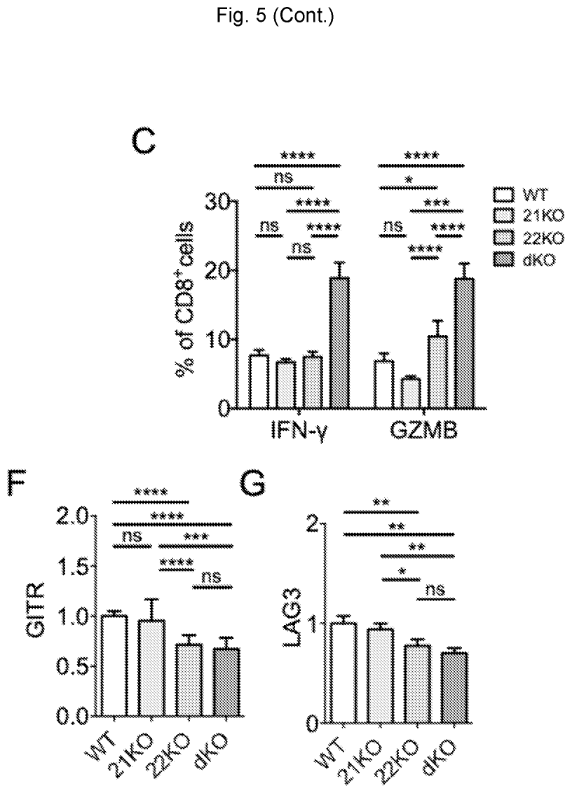

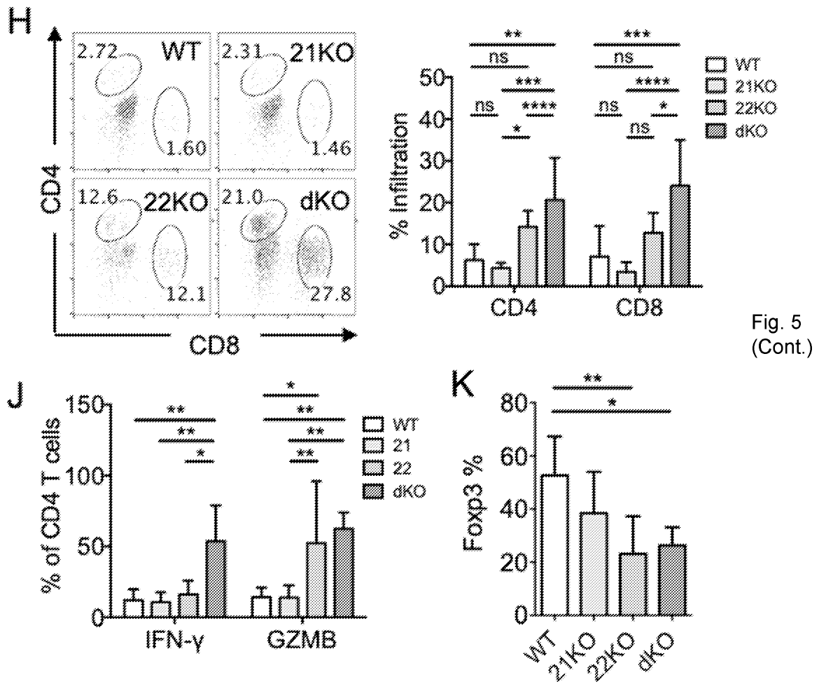

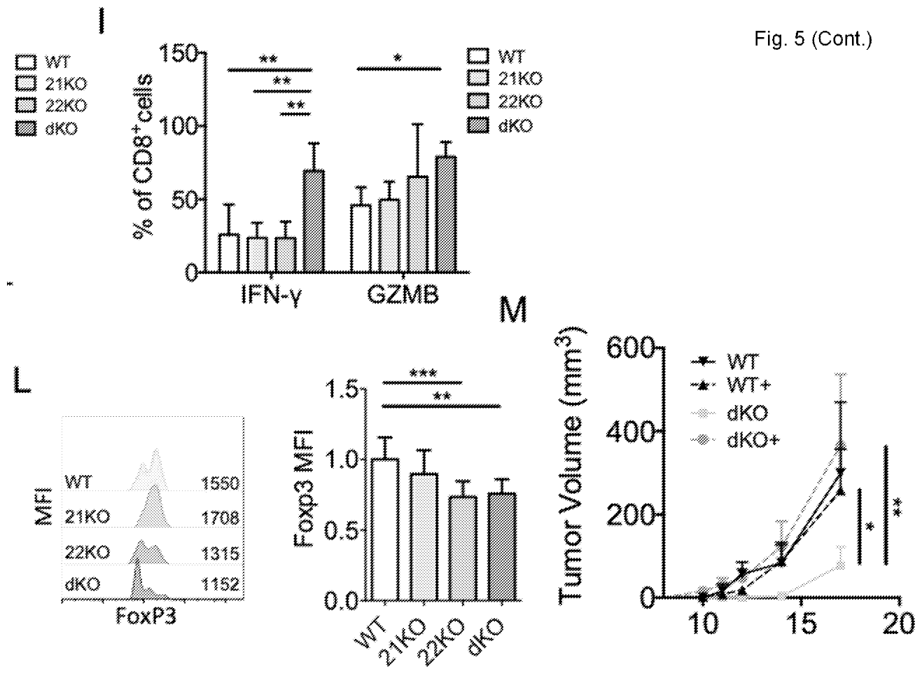

To test the importance of T_{reg} cell Usp22 and Usp21 in tumor conditions in vivo, we used the B16 melanoma syngeneic tumor model. Mice with T_{reg}-specific ablation of Usp22 showed increased tumor rejection compared to the deletion of Usp21. Importantly, though, mice harboring the joint deletion of both Usp22 and Usp21 in T_{reg} cells grew the smallest tumors (FIG. 5A). Additionally, the dKO and 22KO animals showed greater proportions of effector memory CD4⁺ and CD8⁺ T cells in the spleens. In contrast, deletion of Usp21 in T_{reg} cells was insufficient to enhance the B16 tumor rejection (FIG. 5A). Consistently, 21KO mice cytokine levels were on par with WT mice, while the 22KO mice displayed an increase of CD8⁺ Granzyme B (GZMB) production. Notably, the tumor-bearing dKO mice had significant increases of both interferon-γ (IFN-γ) and GZMB producing CD8⁺ T cells in the spleens, and each cytokine was enhanced even in comparison to single KO animals (FIG. 5C). Furthermore, both the 22KO and dKO had significant drops in FOXP3 and T_{reg} suppressive marker MFI in peripheral T_{reg} cells, which was not observed in 21KO T_{reg} cells (FIG. 5D-G). Collectively, these data suggest that the combined loss of Usp21 and Usp22 in T_{reg} cells results in enhanced activation of Teff cells effect compared to individual loss of Usp21 or Usp22 alone.

Further analysis of tumor infiltrating lymphocytes indicated a significant increase in CD4⁺ and CD8⁺ T cell frequencies in the dKO mice, with each compartment in the dKO secreting higher amounts of both IFN-γ and GZMB than WT mice (FIG. 5H-J). Notably, the dKO mice had significantly higher levels of Teff cell infiltration than both the 22KO and the 21KO mice, as well as the having the highest levels of IFN-γ secretion. Consistent with splenic T_{reg} cells, itT_{reg} cells in the 22KO and dKO mice had significantly lower T_{reg} infiltration and FOXP3 MFI than in the WT and 21KO (FIGS. 5K and L). As loss of Usp22 and Usp21 downregulated many T_{reg} suppressive genes, as shown by our RNAseq data (FIG. 4E), we conclude that T_{reg} fragility due to Usp22 and Usp21 loss perpetuates anti-tumor immunity by alleviating T_{reg} suppression on cytotoxic CD8⁺ T cells, shown by the loss of anti-tumor response post-CD8 depletion (FIG. 5N).

Although the loss of USP22 alone displayed significant anti-tumor immunity, the loss of both Usp22 and Usp21 in T_{reg} cells displayed a more vigorous anti-tumor response, as documented by the dKO mice having a dramatically increased cytokine production, the highest infiltrating T cell number, and the smallest tumor sizes. Collectively, this data suggests that Usp21 and Usp22 cooperate to maintain Foxp3 expression and T_{reg} cell function in the TME.

### Identification of a Usp22-Specific Small Molecule Inhibitors

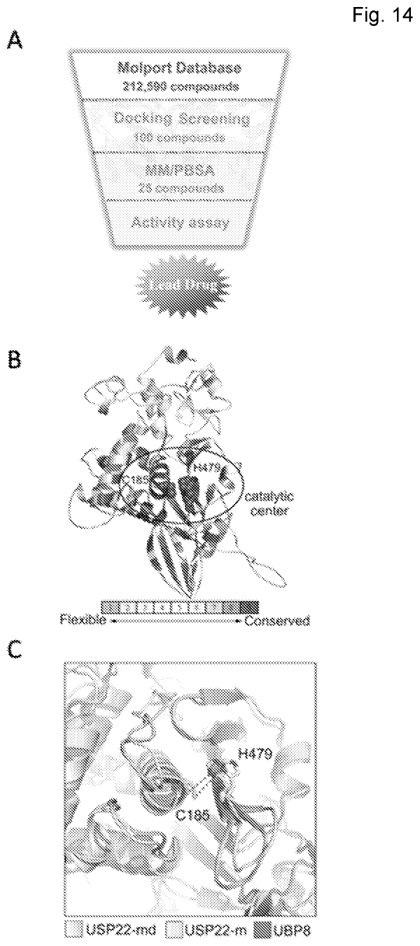

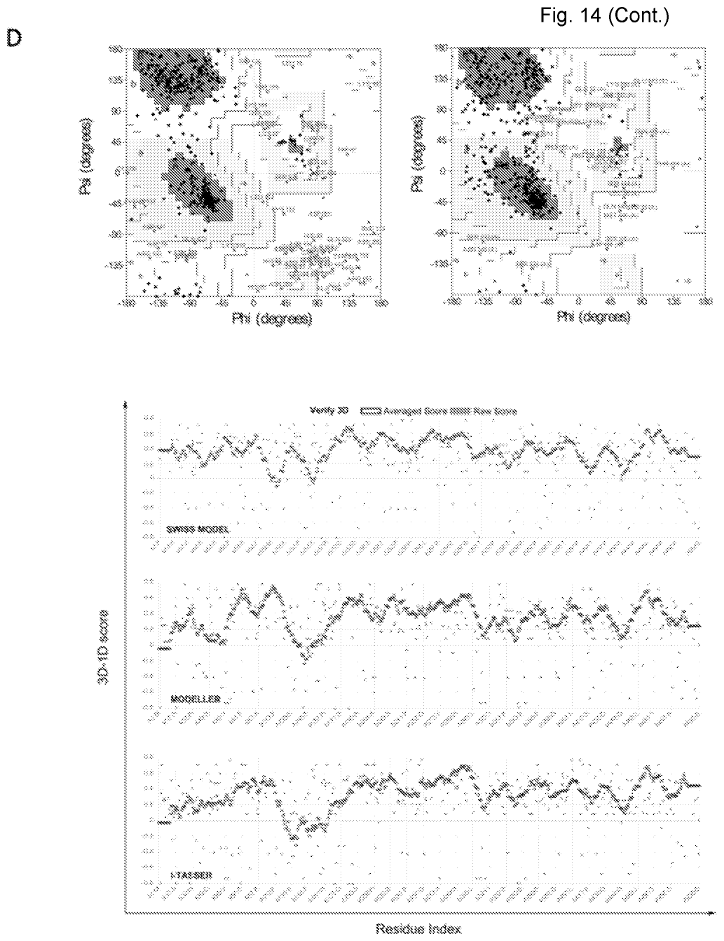

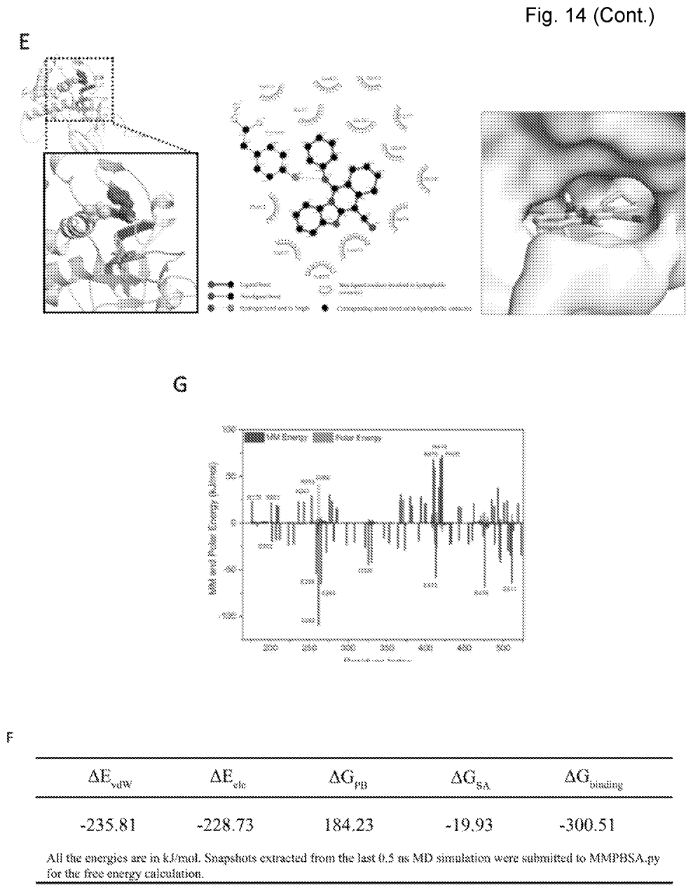

Although deletion of Usp21 in addition to Usp22 in T_{reg} cells enhances antitumor immunity, Usp22 deletion alone is sufficient in diminishing tumor burden. To assess whether pharmacologic inhibition of Usp22 could modulate T_{reg} function, we aimed to identify Usp22-specific inhibitors. It has been suggested that in vitro purified USP22 protein lacks catalytic activity (40, 41), leading to difficulties for high-throughput screening. Therefore, we used the computer-aided drug design (CADD) to develop a Usp22-specific small molecule inhibitor (FIG. 14A). As Usp22 contains a highly conserved putative catalytic domain (Cys, His, and Asp) from yeast to human, a homology modeling study was performed to obtain a model of human Usp22 for use in structure-based virtual screening (FIG. 14B). Of three validated structural models of Usp22, the yeast UBP8 structure (PDB code 3MHS) was chosen as a template protein to construct the Usp22 model by Swiss Model (Usp22-m) (FIGS. 14C and D). In order to obtain conformation at the lowest potential, the structure of Usp22-m was further subjected to molecular dynamics simulation and clustering analysis using Gromacs5.15, and the distance between Cys 185 and His 479 was increased from 3.6 Å to 4.8 Å in the position of catalytic site of USP22 (Usp22-md) (FIG. 14C). We further compared the predicted amino acid sequence of USP22 with 150 homologous full sequences. The conservation grades are mapped onto the structure and show the Cys domain was highly conserved. This study not only provides basis for the accuracy of homology modeling, but also provides favorable conditions for drug selectivity screening.

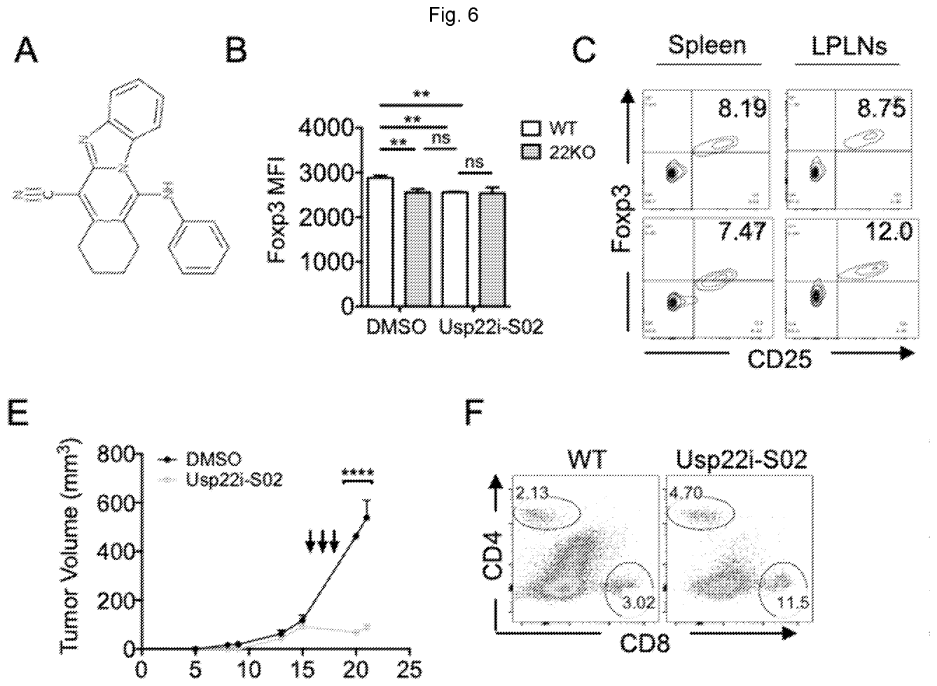

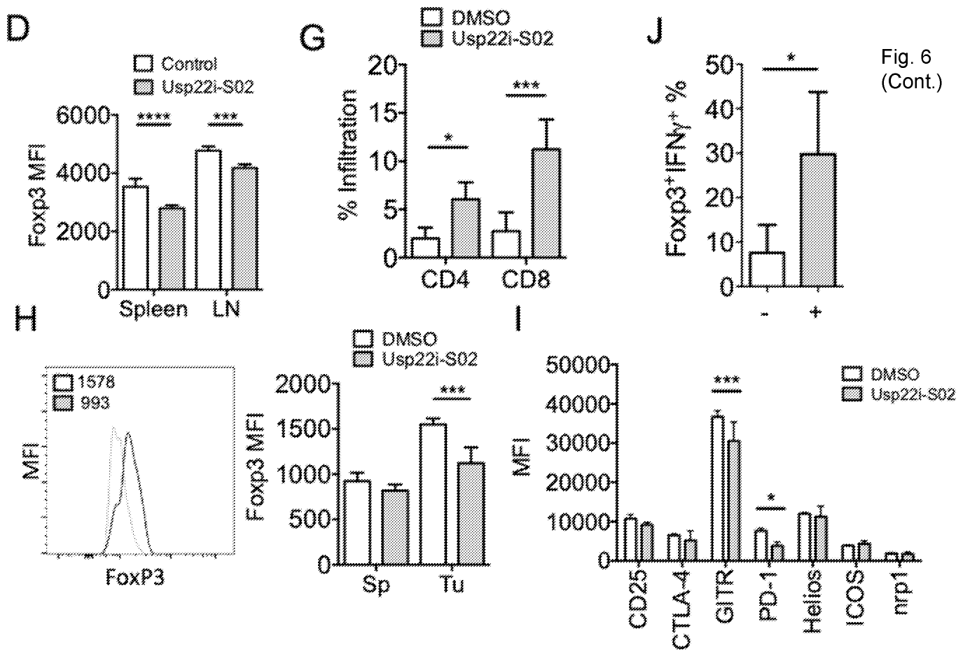

We then used both Lipinski's Rule and Veber's Rule to filter through the Specs database and found a total of 240K compounds binding to the catalytic pocket of our Usp22 model. We then filtered the top 100 compounds ranked by docking affinity by MD and MM/PBSA methods and were left with 25 compounds (Table 1). This limited number of compounds allowed us for further biological screening. As USP22 suppression leads to dramatic reduction in FOXP3 expression levels, we utilized FOXP3 MFI reduction as a readout for the biological validation of USP22 inhibitory efficacy by each of the 25 chemicals. As indicated in Table 1, the chemical S02 (11-anilino-7,8,9,10-tetrahydrobenzimidazo[1,2-b]isoquinoline-6-carbonitrile) showed strong efficacy in downregulating FOXP3 expression. The compound S02, structure shown in FIG. 6A, bound stably in the USP22 catalytic domain pocket shown by the RMSD trajectory (FIG. 14E) with strong binding energies to our USP22-md model (FIG. 14F). Furthermore, analysis of S02 interaction with each residue of Usp22 indicated that the side chain negative residues (Glu, Asp) make a favorable contribution to the binding of the inhibitor and protein, however, the positively charged residues such as Arg and Lys, play a detrimental role (FIG. 14G). Therefore, future generations of Usp22 inhibitors should take into considerations not only the hydrophobic residues, but also the interaction between the inhibitor and the charged and polar residues on the surface of the binding pocket.

### Usp22i-S02 Holds Great Preclinical Efficacy in Enhancing Anti-Tumor Immunity

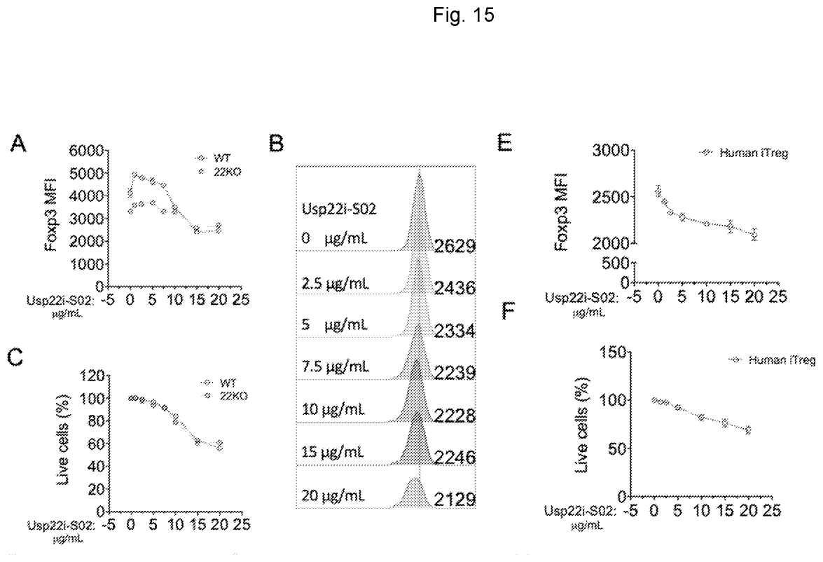

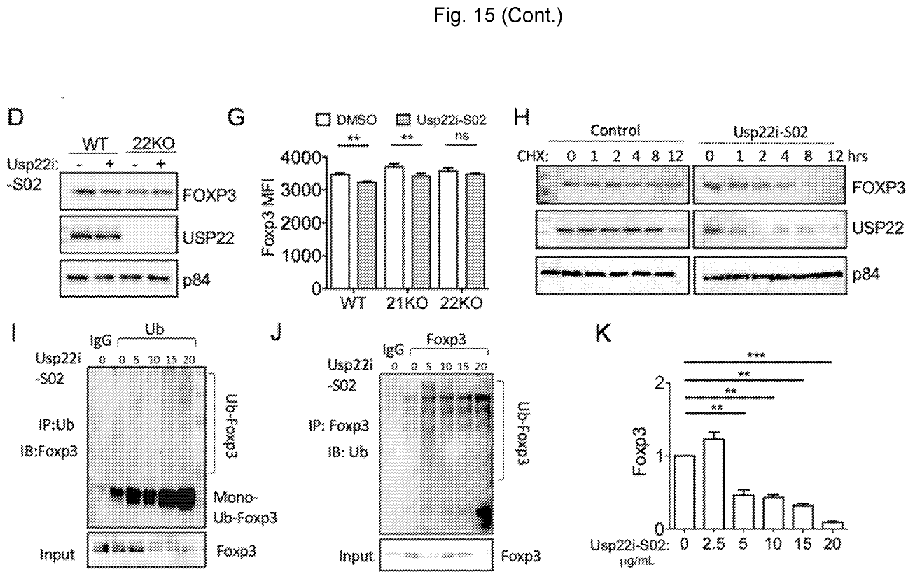

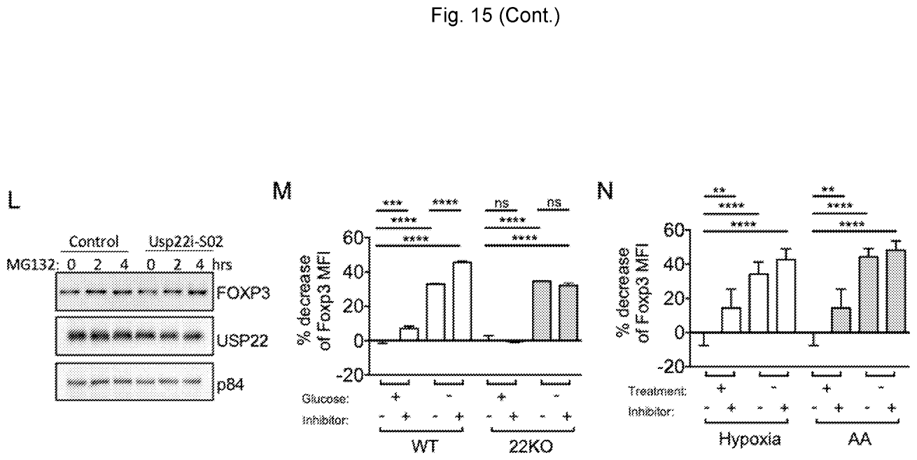

After initial screening, we ran an in vitro dose response study on compound S02, now dubbed Usp22i-S02, in both WT and Usp22-null iT_{reg} cells (FIG. 15A-C). A concentration of 10 μg/mL showed decreases in Foxp3 MFI and protein level comparable to Usp22-null iT_{reg} cells with little effect on viability, indicating a near complete suppression of Usp22 activity in stabilizing Foxp3 (FIG. 15A-D). Importantly, low doses of Usp22i-S02 administration to human T_{reg} cells significantly decreased Foxp3 MFI with little effect to cell viability, showing the relevance of this inhibitor to human cells (FIGS. 15E and F). In contrast, Usp22i-S02 had minimal effect on FOXP3 levels in murine T_{reg} cells already lacking Usp22 both in vivo (FIG. 6B) and in vitro, while having full effect on iT_{reg} cells lacking Usp21 (FIGS. 15A and G). Functionally, Usp22i-S02 administration had similar effects to Usp22 deletion in iT_{reg} cells, resulting in enhanced FOXP3 degradation in cycloheximide (CHX) treated cells, increased FOXP3 ubiquitination, and decreased Foxp3 transcription (FIG. 15H-K). Furthermore, Usp22i-S02-mediated FOXP3 degradation was halted by MG132 protease inhibition, indicating that Usp22i-S02 enhances proteasomal-specific degradation of FOXP3 (FIG. 15L). Importantly, Usp22i-S02 significantly diminished Foxp3 stability in T_{reg} cells under glucose starvation, while having no effect on Foxp3 in T_{reg} cells lacking Usp22 (FIG. 15M). This trend was also seen under hypoxic and amino acid starvation conditions, indicating that Usp22i-S02 reduces Foxp3 stability under THE factors (FIG. 15L). Therefore, these results indicate that Usp22i-S02 is a potent USP22-specific small molecule inhibitor that downregulates Foxp3 expression in T_{reg} cells.

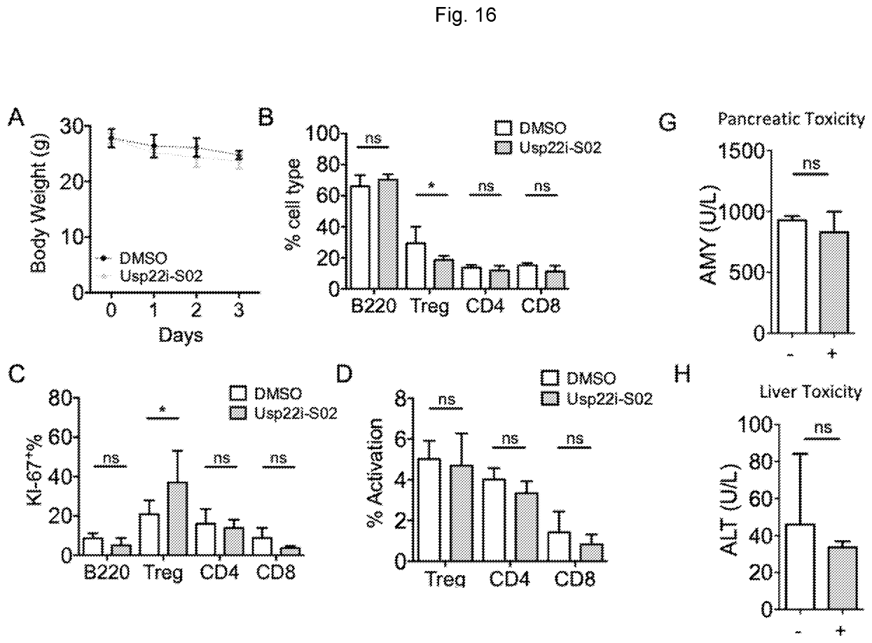

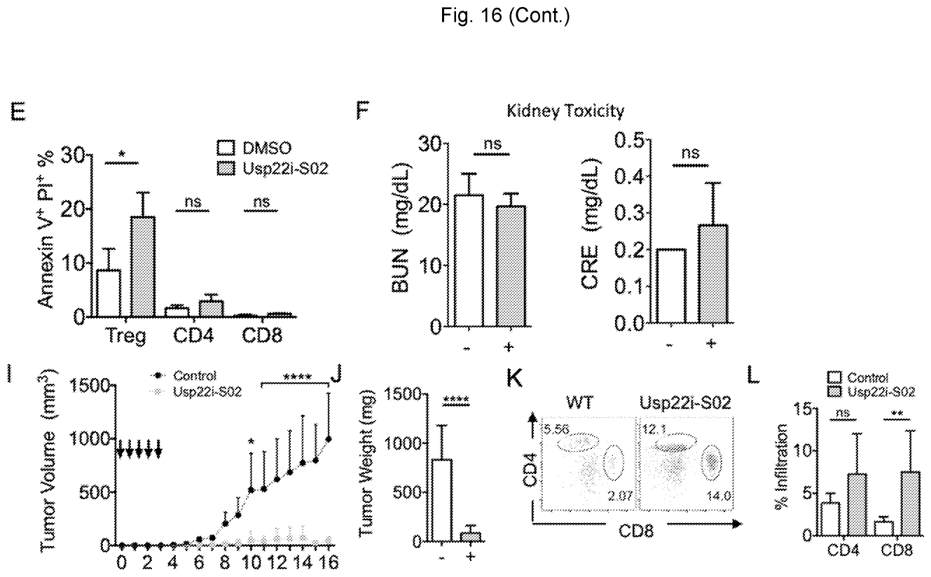

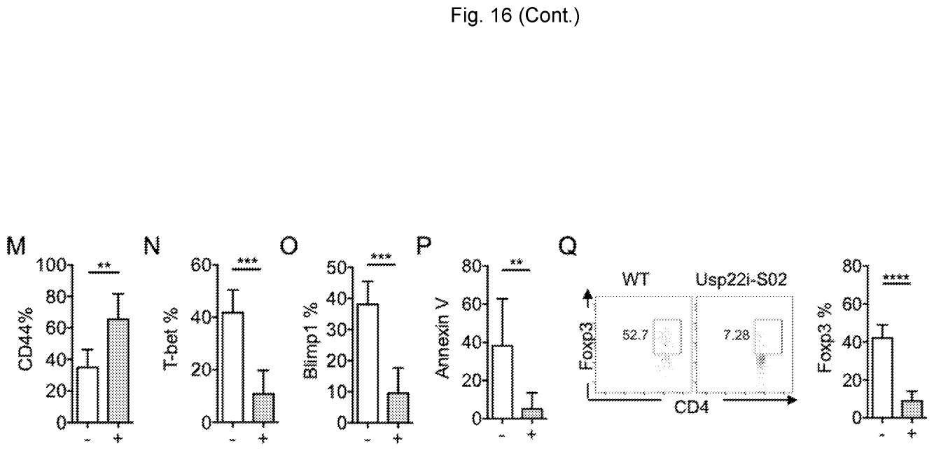

An important aspect of a potential immunotherapeutic is its antitumor functionality paired with low immune toxicity. To determine the toxicity of Usp22i-S02 in vivo, we first determined its effects in naïve mice. We found little alteration in the weights, B cell and Teff cell percentages and proliferation, and Teff cell activation of treated mice compared to DMSO-treated control mice (FIG. 16A-D). Unlike Teff cells, T_{reg} cell death was significantly increased (FIG. 16E), resulting in a decrease in T_{reg} percentage (FIG. 16B). Interestingly, T_{reg} proliferation was also increased (FIG. 16C), potentially indicating a dysfunctional T_{reg} population within the tumor as shown in FIG. 14B. Importantly, administration of Usp22i-S02 to WT mice mimicked a genetic deletion of Usp22 in T_{reg} cells, showing a significant drop in FOXP3 MFI in T_{reg} cells from the spleen and lymph nodes without any alteration in T_{reg} cell frequency, with no additional decrease to FOXP3 MFI in the Usp22-KO mice (FIG. 6B-D). Furthermore, a comprehensive tissue panel showed no organ toxicity differences form control DMSO treated mice (FIG. 16F-H). These data indicate that administration of Usp22i-S02 results in a T_{reg}-specific phenotype in naïve mice with little effects on other immune cell types and tissue toxicity.

To determine the functionality of Usp22i-S02 as a potential therapeutic, we tested the inhibitor on established tumors. Following initial LLC1 tumor establishment, WT mice administered Usp22i-S02 showed striking tumor rejection compared to untreated mice, as well as a significant increase in Teff cell tumor infiltration (FIG. 6E-G). Importantly, intratumoral, but not splenic, Foxp3+ T_{reg} percentage significantly decreased following administration of Usp22i-S02 (FIG. 6H). Furthermore, itT_{reg} cells had lower levels of GITR and PD-1, and also expressed significantly higher levels of IFN-γ, a marker of T_{reg} dysfunction and fragility (42), suggesting the importance of Usp22i-S02 on T_{reg} cells specifically within the tumor (FIG. 6J). Further analysis of tumor infiltrating lymphocytes was done on mice treated immediately following tumor implantation (FIG. 16I). Along with decreased tumor burden and increased Teff cell infiltration, intratumoral CD8⁺ T cells displayed a less exhausted phenotype, with an increase in CD44⁺ cells and a decrease in T-bet⁺, Blimp1⁺, and Annexin V⁺ cells compared to non-treated mice (FIG. 16H-P). Importantly, intratumoral Foxp3+ T_{reg} percentage significantly decreased following administration of Usp22i-S02 (FIG. 16Q).

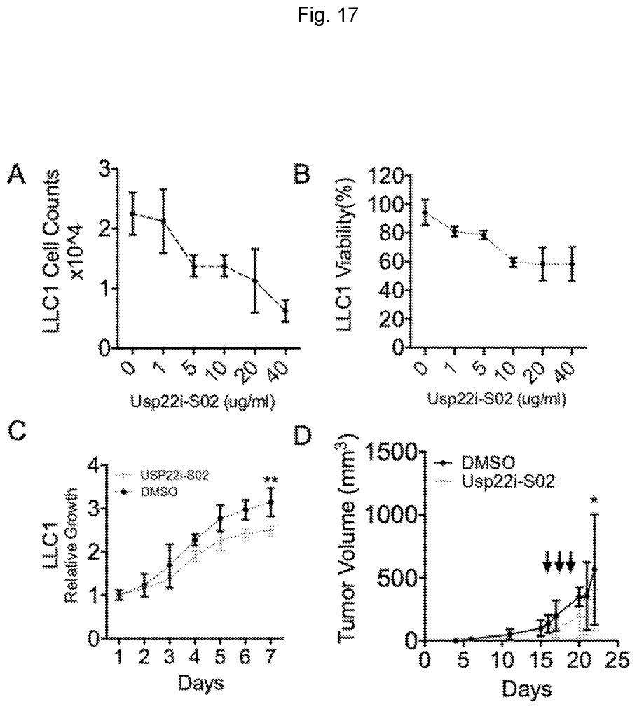

As Usp22 is also an important oncogene (43, 44), we were interested in the potential dual-therapeutic function of Usp22i-S02. Indeed, administration of Usp22i-S02 to LLC1 cells in vitro resulted in decreased tumor cell counts, viability, and growth (FIG. 17A-C). Furthermore, treatment of Rag^{-/-} mice with established tumors resulted in a small but statistically significant decrease in tumor growth, in line with previous observations that tumor cell intrinsic Usp22 is required for tumor growth (FIG. 17D)(45). Together, our data show the critical role of Usp22 in T_{reg} cell stability and adaptation within the TME, and that specifically targeting Usp22 with a small molecule inhibitor enhances anti-tumor immunity through both tumor and immune intrinsic mechanisms.

### Discussion

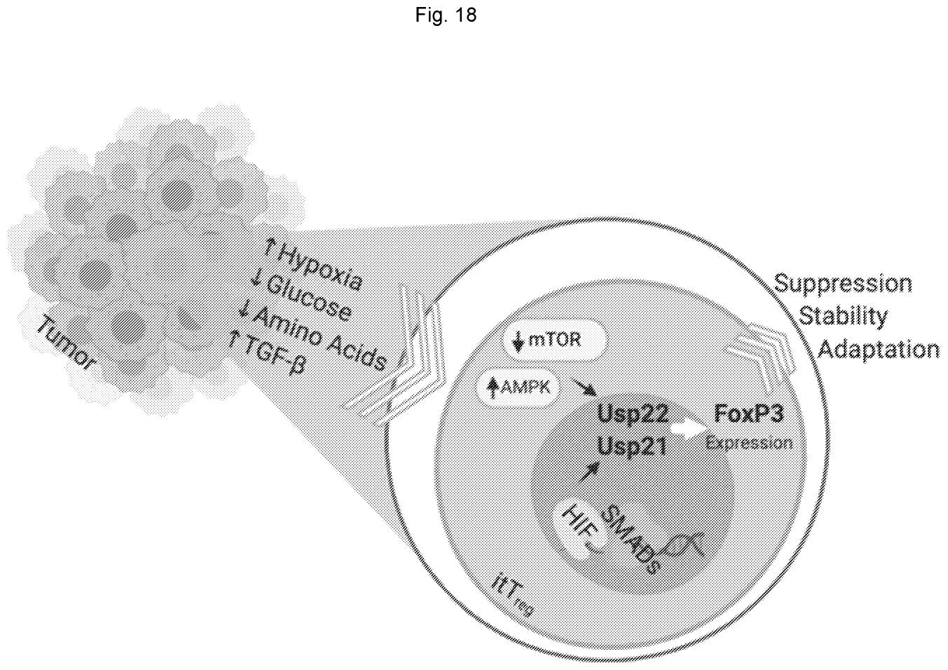

Emerging data suggests that the TME, which is deprived of nutrients and oxygen, likely offers a metabolic advantage to T_{reg} cells over Teff cells to further promote an immunosuppressive microenvironment. However, the TME-specific factors and their cellular targets that potentiate T_{reg} cell suppressive function and adaptation remain largely unidentified. Our study illustrates a previously unappreciated role of Foxp3-specific DUBs, Usp22 and Usp21, as environmentally-sensitive factors that enhance Foxp3 stability in the TME. We identified several TME factors that upregulate Usp22 and Usp21, ultimately stabilizing Foxp3: (1) tumor-secreted TGF-β; (2) hypoxia; (3) glucose-restriction; and (4) amino acid-deprivation (FIG. 18). Our findings unveil new mechanisms behind the metabolic and functional uniqueness of itT_{reg} cells, providing evidence on how these cells adapt in response to environmental cues to support their function.

As it has been well-documented that itT_{reg} cells are more suppressive and often have high Foxp3 expression (9, 10, 46), we first confirmed these findings in various murine tumor models. Interestingly, we found that itT_{reg} cells in these models and in lung cancer patients upregulate Usp22, and sometimes Usp21, when compared to non-tumor-residing T_{reg} cells. Furthermore, Usp22 upregulation is correlated with higher Foxp3 expression in human lung cancer itT_{reg} cells, suggesting that TME factors selectively induce these USPs to protect Foxp3 from ubiquitin-mediated degradation while simultaneously promoting Foxp3 transcription. The fact that we observed increased Usp22 in human itT_{reg} cells broadens the relevance of this pathway to human tumor therapies. Although Usp7 in T_{reg} cells is known to control Foxp3 expression and T_{reg} suppressive function in a model of colitis, we did not observe an increase in Usp7 expression in itTregs, suggesting Usp7 may primarily regulate T_{reg} function during homeostatic conditions.

TGF-β is a major player in iT_{reg} conversion and stability and is broadly secreted by many tumor types. We found that tumor secreted TGF-β is sufficient in upregulating Usp22 through canonical TGF-β signaling. Furthermore, Usp22 partakes in a feedback loop to further upregulate itself and Foxp3 through SMAD protein stabilization. Although Usp21 was not functioning through the canonical TGF-β pathway, it is possible that the non-canonical TGF-β JNK/P38 signaling pathway could be at play (47). As TGF-β is widely implicated in Foxp3 expression and stability, and iT_{reg} function, our data adds a new level of complexity to already known systems (23, 48). These novel mechanisms potentially function to ensure T_{reg} cell stabilization through alternate pathways, strengthening their ability to maintain their suppressive capacity in diverse microenvironments.

However, tumor-secreted TGF-β is not the only factor capable of upregulating USPs, since T_{reg} cells treated with EG7 TCM could not recapitulate the increase of Usp22 seen in itT_{reg} cells isolated from EG7 tumors. Therefore, we hypothesized that other environmental factors within the TME are also implicated in T_{reg} stabilization through USPs. As hypoxia is a major hallmark of solid tumors (3, 29), we investigated how low oxygen conditions influence Usp22 levels in T_{reg} cells. Hypoxia induced Usp22 in a HIF-dependent manner. Also, upon Usp22 deletion, nT_{reg} cells under hypoxic stress could not sustain stable FOXP3 expression. Our findings are in line with previous data that demonstrated heightened proliferation and suppressive capabilities of nT_{reg} cells under hypoxic conditions (27). These data, paired with the knowledge of two functioning HIF binding sites along the Usp22 promoter, imply that hypoxia can enhance T_{reg} suppressive function through Usp22-dependent stabilization of FOXP3 (33).

Along with a decrease in oxygen availability, the competition for nutrients that occurs within the TME influences immune cell growth, survival, and function. Classically, T_{reg} cells are thought to have a significantly lower reliance on glycolysis than Teff cells, potentially providing another advantage (5, 34, 37). Our data identifies Usp22 as an important mediator in this process, functioning to stabilize FOXP3 under glucose- and amino acid-deprivation. In part the enhanced stability of FOXP3 appears secondary to AMPK activation, which likely occurs under glucose restriction within the TME. Interestingly, AMPK activation in T_{reg} cells is accompanied by a shift towards oxidative metabolism, which may further enhance T_{reg} survival in the TME (49). We show that AMPK activation is sufficient to upregulate Usp22 and Usp21, implicating their involvement in FOXP3 stabilization for T_{reg} cell function under energy stress. The promotion of AMPK signaling via nutrient deficiency also suppresses mTOR activity within T cells (35, 50). As the balance of AMPK and mTOR signaling functions as an environmental sensor for nutrient availability, it is possible that AMPK activation primarily increases Usp22 and Usp21 expression thru inhibition of mTOR signaling. Indeed, mTOR inhibition was capable of upregulating Usp22 and Usp21 in T_{reg} cells.

The metabolic status of an immune cell is highly important within the TME for their cell survival and function. As T_{reg} cells can adapt to low-oxygen, low nutrient environments, this gives them a metabolic advantage compared to Teff cells. Importantly, FOXP3 is essential to this process as it is known to promote oxidative phosphorylation within T_{reg} cells. We show that Usp22- and Usp21-deficient T_{reg} cells have significantly altered expression of metabolic genes and impaired OCR and ECAR. In addition, RNA sequencing analysis demonstrated that loss of Usp22 and Usp21 in T_{reg} cells resulted in the upregulation of multiple pathways associated with cell growth and proliferation. Collectively, these data raise the intriguing possibility that Usp22 and Usp21 work to promote T_{reg} cell quiescence in nutrient-restricted environments in part through modulating T_{reg} cell metabolic programs. Together, our data indicate that microenvironmental stress within the TME upregulates T_{reg} USP levels, which then function to stabilize FOXP3. Enhanced FOXP3 stability further supports T_{reg} cell adaptation to the TME; thus, identifying Usp22 and Usp21 as important environment-sensitive factors that regulate T_{reg} cell identity, metabolism and function in the TME.

Additionally, we and others have demonstrated that both Usp22 and Usp21 are upregulated in many cancer types, such as gastric carcinoma, pancreatic cancer and melanoma, and have been correlated with poor prognosis (51, 52). Usp22 promotes oncogenic c-Myc activation as well as indirectly antagonizes the tumor suppressive function of p53, while Usp21 functions as an oncogene by stabilizing a group of transcription factors including Fra1, FoxM1 and Wnt (52-54). Importantly, Usp22 and Usp21 also function to maintain Foxp3 expression through DUB function at the transcriptional (Usp22) and post-translational (both) levels. This duality makes Usp22 and Usp21 highly attractive potential therapeutics that can target both tumor cell intrinsic and immunosuppressive pathways simultaneously. Indeed, their combined loss resulted in the most significant impairment in T_{reg} tumor-promoting functions, suggesting that Usp22 and Usp21 play distinct roles in modulating T_{reg} cell adaption and function in the TME.

However, loss of Usp22 in T_{reg} cells resulted in enhanced anti-tumor immunity relative to the loss of Usp21, suggesting a dominance of Usp22 in itT_{reg} cells. Therefore, specifically targeting Usp22 may be sufficient in eliminating the advantage T_{reg} cells have over T_{eff} Within the TME. To test this, we developed and tested the first ever Usp22-specific inhibitor. Administration of the inhibitor resulted in a dramatic decrease in itT_{reg} number, resulting in strong in vivo anti-tumor effects. Our data demonstrate that Usp22 is a targetable protein, and that the inhibitor Usp22i-S02 has the potential of being incorporated into tumor immune therapies. Furthermore, many current therapeutics focus on promoting Teff cell function, as such the addition of Usp22 inhibition with current therapies could further enhance anti-tumor immunity through synergistic effects.

### Materials and Methods

### Tumor Models

EG7 Lymphoma, LLC 1 lung carcinoma, and B 16-F10 melanoma cell lines were provided by the Zhang laboratory at Northwestern and used for tumor models as previously reported (14). The cells lines were cultured in DMEM with 10% FBS, and were tested for mycoplasma using LookOut Mycoplasma PCR detection kit (Sigma, MP0035-1KT). Cultured cancer cells were trypsinized and washed once with PBS. LLC1 lung carcinoma tumor cells were subcutaneously administered to the right flank of 8- to 10-week-old mice at 1×10⁶ tumor cells per mouse, and B16 melanoma at 5×10⁴ tumor cells per mouse. Tumors were measured every 2-3 days by measuring along 3 orthogonal axes (x, y and z) and tumor volume was calculated as (xyz)/2. The tumor size limit agreed by IRB was 2 cm³.

In Vitro iT_{reg} Cell TCM and TGF-β Assays

Previously generated iT_{reg} cells were washed and rested for 7 hours in OPTImem media containing 5 ng/ml of IL-2 to maintain survival. OPTImem was used to avoid any TGF-β contamination found in serum. After resting, the cells were incubated in OPTImem containing IL-2 with or without the addition of 20 ng/ml TGF-β or the various tumor cell medias (B16, LLC1, and EG7). TCM was obtained by plating B16, EG7, or LLC1 cell lines at 50% confluency for 16 hours. TCM was then mixed 50:50 with fresh OPTImem and incubated on iT_{reg} cells for 24 hours. TGF-β inhibitor LY 3200882 (Med Chem Express: Cat. No.: HY-103021) was added at 25 μg/mL where indicated.

### In Vitro T_{reg} Cell Hypoxia Culture

nT_{reg} cells were isolated as described above and cultured at 37° C. in either normoxic (21% O₂) or in a hypoxic condition (1% O₂) for 24 hours. Hypoxia was induced using (Name of hypoxia chamber and company). T cell medium was incubated at 37° C. at normoxia or hypoxia for 3 hours prior to usage. Cells were then collected and RNA was extracted as described above. For iT_{reg} cells, cells were isolated and polarized as described above. Subsequently, cells were rested in optiMEM overnight and then plated in optiMEM containing 5 ng/ml IL-2 in either normoxic or hypoxic conditions. optiMEM media was incubated at 37° C. at normoxia or hypoxia overnight prior to usage. Hypoxia stability assay was conducted as described above but cells were cultured in normoxia or hypoxia for 72 hours, then collected and stained for FOXP3 for flow cytometry.

### Glucose and Amino Acid Restriction Assays

nT_{reg} cells were isolated as described above and cultured in either normal T cell medium, T cell medium lacking glucose (Thermo Fisher Catalog #11879020), or T cell medium lacking amino acids including glutamine (US Biological Catalog #R9010-02) substituted with dialyzed FBS (GIBCO Catalog #A3382001) for 24 hours at 1×10⁵ cells per well. T cell media included with 2000U of IL-2 and CD3/CD28 beads as described above. iT_{reg} cells were isolated and polarized as described above for 3 days. Following polarization, iT_{reg} cells were cultured in normal T cell media or T cell media lacking glucose or amino acids for 24 hours. Both nT_{reg} and iT_{reg} cells were then collected and RNA was extracted as described above. For stability assays, cells were cultured as described above for 48 hours, then collected and stained for FOXP3 for flow cytometry.

### In Vitro Inhibitor Assays

All nT_{reg} and iT_{reg} cells were plated as described above DMOG (Sigma Catalog #D3695) was administered to the cells in relevant experiments at 1 mM for 24 hours. Oligomycin (Sigma Catalog #75351) was administered at 1 μM to the media of the cells in relevant experiments for 24 hours. Torin 1 (Millipore Catalog #475991) was administered to the relevant cells at 250 nM for 24 hours. FOXP3 protein level was assessed via flow cytometry following 48 hours of treatment of inhibitors described above. In vitro administration of Usp22i-S02 was at 10 ug/mL.

### Usp22i-S02 In Vivo Inhibitor Experiments.

LLC 1 cells were transplanted into 6-to-8-week-old C57BL/6 male mice. Subcutaneous injections were performed in the right flank of mice in a final volume of 100 μL using 1\hat{ }6 cells per injection. The USP22i-S02 was injected intraperitoneally (i.p) at a concentration of 20 mg/kg/time, in 100 μL of oil, twice a day for 5 days beginning on the day of the LLC1 cells injection. Control animals received 100 μL of oil alone. Subcutaneous tumor diameters were measured daily with calipers until any tumor in the mouse cohort reached 2.5 cm in its largest diameter. Cells were processed and analyzed as stated above.

### Statistics and Data Availability

No statistical methods were used to predetermine sample size. The experiments were not randomized. The investigators were not blinded to allocation during experiments and outcome assessment. All statistical analyses were computed with GraphPad and tests used for each experiment are listed in the fig. legends. ANOVAs with multiple comparisons between rows were corrected with Tukey's test to determine statistical significance. Two-tailed unpaired t tests were performed with Welch's correction.

### REFERENCES

1. T. J. Curiel, et al., Specific recruitment of regulatory T cells in ovarian carcinoma fosters immune privilege and predicts reduced survival. Nat Med 10, 942-949 (2004).
2. G. P. Dunn, A. T. Bruce, H. Ikeda, L. J. Old, R. D. Schreiber, Cancer immunoediting: from immunosurveillance to tumor escape. Nat Immunol 3, 991-998 (2002).
3. D. Hanahan, R. Weinberg, Hallmarks of Cancer: The Next Generation. Cell 144, 646-674 (2011).
4. M. De Simone, et al., Transcriptional Landscape of Human Tissue Lymphocytes Unveils Uniqueness of Tumor-Infiltrating T Regulatory Cells. Immunity 45, 1135-1147 (2016).
5. A. Angelin, et al., Foxp3 Reprograms T Cell Metabolism to Function in Low-Glucose, High-Lactate Environments. Cell Metab 25, 1282-1293.e7 (2017).
6. C. M. Paluskievicz, et al., T Regulatory Cells and Priming the Suppressive Tumor Microenvironment. Front Immunol 10, 2453 (2019).
7. N. E. Scharping, et al., The Tumor Microenvironment Represses T Cell Mitochondrial Biogenesis to Drive Intratumoral T Cell Metabolic Insufficiency and Dysfunction. Immunity 45, 374-388 (2016).
8. G. Deng, Tumor-infiltrating regulatory T cells: origins and features. Am J Clin Exp Immunol 7, 81-87 (2018).
9. M. De Simone, et al., Transcriptional Landscape of Human Tissue Lymphocytes Unveils Uniqueness of Tumor-Infiltrating T Regulatory Cells. Immunity 45, 1135-1147 (2016).
10. G. Plitas, et al., Regulatory T Cells Exhibit Distinct Features in Human Breast Cancer. Immunity 45, 1122-1134 (2016).
11. J. D. Fontenot, M. A. Gavin, A. Y. Rudensky, Foxp3 programs the development and function of CD4+CD25+ regulatory T cells. Nat Immunol 4, 330-336 (2003).
12. P. Attia, A. V. Maker, L. R. Haworth, L. Rogers-Freezer, S. A. Rosenberg, Inability of a Fusion Protein of IL-2 and Diphtheria Toxin (Denileukin Diftitox, DAB389IL-2, ONTAK) to Eliminate Regulatory T Lymphocytes in Patients With Melanoma. J Immunother 28, 582-592 (2005).
13. A. Tanaka, S. Sakaguchi, Targeting T_{reg} cells in cancer immunotherapy. Eur J Immunol 49, 1140-1146 (2019).
14. J. Barbi, D. M. Pardoll, F. Pan, Ubiquitin-dependent regulation of Foxp3 and T_{reg} function. Immunol Rev 266, 27-45 (2015).
15. E. Montauti, D. Fang, Regulation of T_{reg} Functions by the Ubiquitin Pathway. Adv Exp Med Biol 1278, 47-62 (2020).
16. J. T. Cortez, et al., CRISPR screen in regulatory T cells reveals modulators of Foxp3. Nature 582, 416-420 (2020).
17. L. Wang, et al., Ubiquitin-specific Protease-7 Inhibition Impairs Tip60-dependent Foxp3+ T-regulatory Cell Function and Promotes Antitumor Immunity. Ebiomedicine 13, 99-112 (2016).
18. Y. Li, et al., USP21 prevents the generation of T-helper-1-like T_{reg} cells. Nat Commun 7, 13559 (2016).
19. C. A. Crane, B. J. Ahn, S. J. Han, A. T. Parsa, Soluble factors secreted by glioblastoma cell lines facilitate recruitment, survival, and expansion of regulatory T cells: implications for immunotherapy. Neuro-oncology 14, 584-595 (2012).
20. M. D. Wellenstein, K. E. de Visser, Cancer-Cell-Intrinsic Mechanisms Shaping the Tumor Immune Landscape. Immunity 48, 399-416 (2018).
21. Z. Yang, et al., Notch1 signaling in melanoma cells promoted tumor-induced immunosuppression via upregulation of TGF-β1. JExp Clin Canc Res 37, 1(2018).
22. K. de Visser, W. Kast, Effects of TGF-β on the immune system: implications for cancer immunotherapy. Leukemia 13, 1188-1199 (1999).
23. S. Fu, et al., TGF-beta Induces Foxp3+ T-Regulatory Cells from CD4+CD25− Precursors. Am J Transplant 4, 1614-1627 (2004).
24. T. Takimoto, et al., Smad2 and Smad3 Are Redundantly Essential for the TGF-β-Mediated Regulation of Regulatory T Plasticity and Th1 Development. J Immunol 185, 842-855 (2010).
25. L. Lu, et al., Synergistic effect of TGF-β superfamily members on the induction of Foxp3+ T_{reg}. Eur J Immunol 40, 142-152 (2010).
26. S. Dennler, S. Huet, J.-M. Gauthier, A short amino-acid sequence in MH1 domain is responsible for functional differences between Smad2 and Smad3. Oncogene 18, 1643-1648 (1999).
27. A. M. Westendorf, et al., Hypoxia Enhances Immunosuppression by Inhibiting CD4+ Effector T Cell Function and Promoting T_{reg} Activity. Cell Physiol Biochem 41, 1271-1284 (2017).
28. V. Kumar, D. I. Gabrilovich, Hypoxia-inducible factors in regulation of immune responses in tumour microenvironment. Immunology 143, 512-519 (2014).
29. S. Chouaib, M. Z. Noman, K. Kosmatopoulos, M. A. Curran, Hypoxic stress: obstacles and opportunities for innovative immunotherapy of cancer. Oncogene 36, 439-445 (2017).
30. G. L. Semenza, Targeting HIF-1 for cancer therapy. Nat Rev Cancer 3, 721-732 (2003).
31. E. T. Clambey, et al., Hypoxia-inducible factor-1 alpha-dependent induction of FoxP3 drives regulatory T-cell abundance and function during inflammatory hypoxia of the mucosa. Proc National Acad Sci 109, E2784-E2793 (2012).
32. B. Keith, R. S. Johnson, M. C. Simon, HIF1α and HIF2α: sibling rivalry in hypoxic tumour growth and progression. Nat Rev Cancer 12, 9-22 (2012).
33. T. H. Kim, et al., Gα12 ablation exacerbates liver steatosis and obesity by suppressing USP22/SIRT1-regulated mitochondrial respiration. J Clin Invest 128, 5587-5602 (2018).
34. K. E. Beckermann, S. O. Dudzinski, J. C. Rathmell, Dysfunctional T cell metabolism in the tumor microenvironment. Cytokine Growth FR 35, 7-14 (2017).
35. C.-H. Chang, et al., Metabolic Competition in the Tumor Microenvironment Is a Driver of Cancer Progression. Cell 162, 1229-1241 (2015).
36. R. D. Michalek, et al., Cutting Edge: Distinct Glycolytic and Lipid Oxidative Metabolic Programs Are Essential for Effector and Regulatory CD4+ T Cell Subsets. J Immunol 186, 3299-3303 (2011).
37. S. E. Weinberg, et al., Mitochondrial complex III is essential for suppressive function of regulatory T cells. Nature 565, 495-499 (2019).
38. S. P. Cobbold, et al., Infectious tolerance via the consumption of essential amino acids and mTOR signaling. Proc National Acad Sci 106, 12055-12060 (2009).
39. L. A. J. O'Neill, D. G. Hardie, Metabolism of inflammation limited by AMPK and pseudo-starvation. Nature 493, 346-355 (2013).
40. A. Kohler, E. Zimmerman, M. Schneider, E. Hurt, N. Zheng, Structural Basis for Assembly and Activation of the Heterotetrameric SAGA Histone H2B Deubiquitinase Module. Cell 141, 606-617 (2010).
41. M. T. Morgan, et al., Structural basis for histone H2B deubiquitination by the SAGA DUB module. Science 351, 725-728 (2016).
42. A. E. Overacre-Delgoffe, et al., Interferon-γ Drives T_{reg} Fragility to Promote Anti-tumor Immunity. Cell 169, 1130-1141.e11 (2017).
43. J. Melo-Cardenas, Y. Zhang, D. D. Zhang, D. Fang, Ubiquitin-specific peptidase 22 functions and its involvement in disease. Oncotarget 7, 44848-44856 (2016).
44. R. S. Schrecengost, et al., USP22 Regulates Oncogenic Signaling Pathways to Drive Lethal Cancer Progression. Cancer Res 74, 272-286 (2014).
45. L. Lv, et al., Silencing USP22 by asymmetric structure of interfering RNA inhibits proliferation and induces cell cycle arrest in bladder cancer cells. Mol Cell Biochem 346, 11-21 (2011).
46. C. Procaccini, et al., The Proteomic Landscape of Human Ex Vivo Regulatory and Conventional T Cells Reveals Specific Metabolic Requirements. Immunity 44, 406-421 (2016).
47. M. E. Engel, M. A. McDonnell, B. K. Law, H. L. Moses, Interdependent SMAD and JNK Signaling in Transforming Growth Factor-β-mediated Transcription. J Biol Chem 274, 37413-37420 (1999).
48. S. Budhu, et al., Blockade of surface-bound TGF-β on regulatory T cells abrogates suppression of effector T cell function in the tumor microenvironment. Sci Signal 10, eaak9702 (2017).
49. G. A. Gualdoni, et al., The AMP analog AICAR modulates the Treg/Th17 axis through enhancement of fatty acid oxidation. Faseb J 30, 3800-3809 (2016).
50. N. M. Chapman, H. Chi, mTOR signaling, Tregs and immune modulation.

Immunotherapy 6, 1295-1311 (2014).
51. G. V. Glinsky, O. Berezovska, A. B. Glinskii, Microarray analysis identifies a death-from-cancer signature predicting therapy failure in patients with multiple types of cancer. J Clin Invest 115, 1503-1521 (2005).
52. P. Hou, et al., USP21 deubiquitinase promotes pancreas cancer cell sternness via Wnt pathway activation. Gene Dev 33, 1361-1366 (2019).
53. Z. Lin, et al., USP22 Antagonizes p53 Transcriptional Activation by Deubiquitinating Sirt1 to Suppress Cell Apoptosis and Is Required for Mouse Embryonic Development. Mol Cell 46, 484-494 (2012).
54. L. Peng, et al., Ubiquitin specific protease 21 upregulation in breast cancer promotes cell tumorigenic capability and is associated with the NOD-like receptor signaling pathway. Oncol Lett 12, 4531-4537 (2016).

### CONCLUSION

Our studies identify 11-anilino-7,8,9,10-tetrahydrobenzimidazo[1,2-b]isoquinoline-6-carbonitrile, or USP22i-S02, as a USP22-specific inhibitor. This inhibitor appears to be an ideal antitumor therapeutic drug because: (i) it inhibits T_{reg} suppressive functions and (ii) inhibit tumor cell expression of PD-L 1, both of which enhances antitumor immune response. In addition, (iii) USP22i-S02 can directly inhibit tumor cell proliferation through USP22 suppression.

TABLE 1
Compounds

| | | MM/PBSA | | Efficacy |
|---|---|---|---|---|
| | | Binding Free | | in |
| Chemical | | Energy | | USP22 |
| number | Chemical name | (kJ/mol) | Chemical structure | inhibition |
| S01 | 7-(difluoromethyl)- N-(3,4- dimethylphenyl)- 5-phenylpyrazolo [1,5-a]pyrimidine- 3-carboxamide | −342.23 | 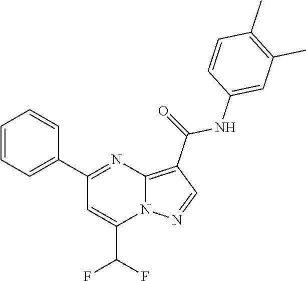 [SMILES: Cc1ccc(NC(=O)c2cnn3c(C(F)F)cc(-c4ccccc4)nc23)cc1C] | − |
| S02 | 11-anilino- 7,8,9,10- tetrahydro- benzimidazo [1,2-b] isoquinoline- 6-carbonitrile | −300.51 | 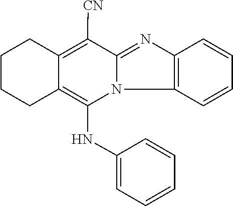 [SMILES: N#Cc1c2c(c(Nc3ccccc3)n3c1nc1ccccc13)CCCC2] | + |
| S03 | 2,7-bis(4- methoxyphenyl) 9-oxo-9H- fluorene- 2,7-disulfonate | −201.50 | 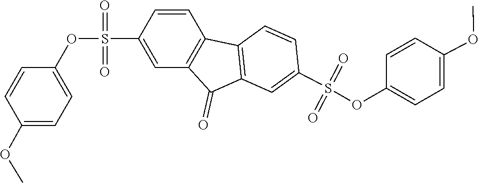 [SMILES: COc1ccc(OS(=O)(=O)c2ccc3c(c2)C(=O)c2cc(S(=O)(=O)Oc4ccc(OC)cc4)ccc2-3)cc1] | − |
| S04 | 6-(2,5- dimethoxyphenyl)- 2-oxo-1,2- dihydropyridine-3- carbonitrile | −190.83 | 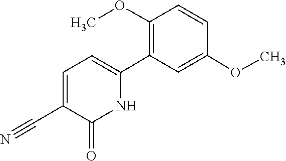 [SMILES: COc1ccc(OC)c(-c2ccc(C#N)c(=O)[nH]2)c1] | − |
| S05 | 2,4- dimethanesulfonyl- 8-methoxy-5H,6H- benzo[h]quinazoline | −187.88 | 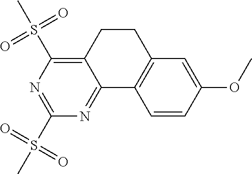 [SMILES: COc1ccc2c(c1)CCc1c-2nc(S(C)(=O)=O)nc1S(C)(=O)=O] | − |
| S06 | 4,5-bis(4- methoxyphenoxy) benzene-1,2- dicarbonitrile | −175.87 | 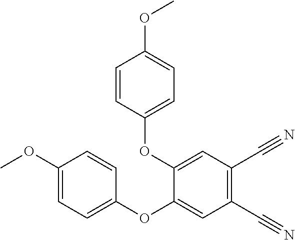 [SMILES: COc1ccc(Oc2cc(C#N)c(C#N)cc2Oc2ccc(OC)cc2)cc1] | − |
| S07 | 9-[(3-methylbut-2- en-1-yl)oxy]- 7H-furo[3,2-g] chromen-7-one | −164.09 | 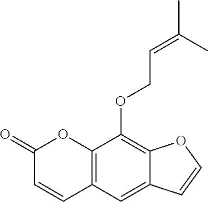 [SMILES: CC(C)=CCOc1c2occc2cc2ccc(=O)oc12] | − |
| S08 | N-(2-{[5- (ethanesulfonyl)-3- nitrothiophen-2- yl]sulfanyl}phenyl) acetamide | −147.32 | 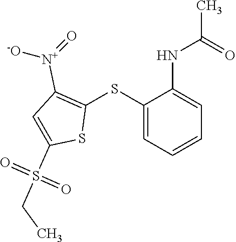 | − |
| S09 | 1-[4-nitro- 5-(pyridin-4- ylsulfanyl)thiophen- 2-yl]ethan-1- one | −141.38 | 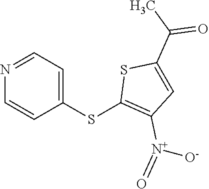 [SMILES: CC(=O)c1cc([N+](=O)[O-])c(Sc2ccncc2)s1] | − |
| S10 | bis[(4- methoxyphenyl) amino]pyrazine- 2,3-dicarbonitrile | −139.07 | 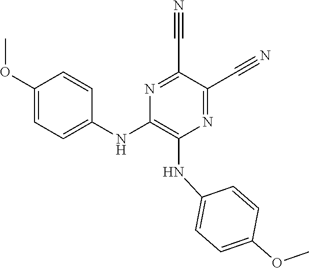 [SMILES: COc1ccc(Nc2nc(C#N)c(C#N)nc2Nc2ccc(OC)cc2)cc1] | − |
| S11 | 5-{[(2,4- dimethylphenyl) sulfonyl]amino}- 2-methyl-N- phenylnaphtho[1,2- b]furan-3- carboxamide | −134.24 | 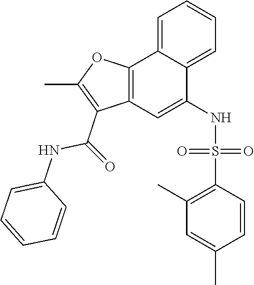 [SMILES: Cc1ccc(S(=O)(=O)Nc2cc3c(C(=O)Nc4ccccc4)c(C)oc3c3ccccc23)c(C)c1] | − |
| S12 | 8- Oxotetrahydro- palmatine | −133.44 | 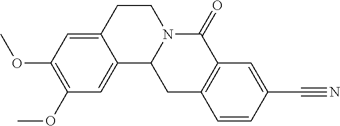 [SMILES: COc1cc2c(cc1OC)C1Cc3ccc(C#N)cc3C(=O)N1CC2] | − |
| S13 | 1-{5-[(4- chlorophenyl) amino]-4- nitrothiophen-2- yl}ethan-1-one | −121.93 | 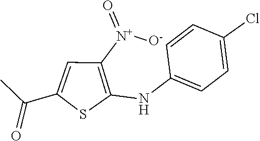 [SMILES: CC(=O)c1cc([N+](=O)[O-])c(Nc2ccc(Cl)cc2)s1] | − |
| S14 | ethyl 6-cyano-7-(4- methoxyphenyl)-5- oxo-1-phenyl-1,5- dihydro[1,2,4] triazolo[4,3-a] pyrimidine-3- carboxylate | −119.36 | 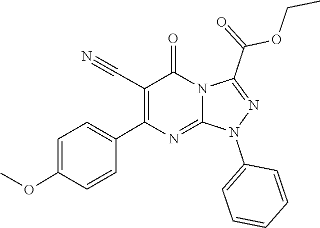 [SMILES: CCOC(=O)c1nn(-c2ccccc2)c2nc(-c3ccc(OC)cc3)c(C#N)c(=O)n12] | - |
| S15 | 1-(5-{[(4- chlorophenyl)methyl] sulfanyl}-4- nitrothiophen-2- yl)ethan-1-one | −118.92 | 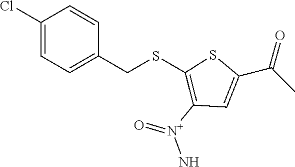 [SMILES: CC(=O)c1cc([N+](N)=O)c(SCc2ccc(Cl)cc2)s1] | − |
| S16 | bis[(3- chlorophenyl)amino] pyrazine- 2,3-dicarbonitrile | −117.91 | 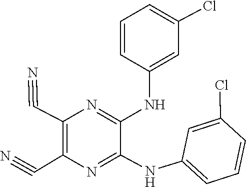 [SMILES: N#Cc1nc(Nc2cccc(Cl)c2)c(Nc2cccc(Cl)c2)nc1C#N] | − |
| S17 | 1-{5-[(4- methoxyphenyl) sulfanyl]-4- nitrothiophen-2- yl}ethan-1-one | −115.11 | 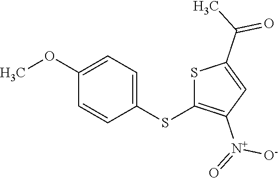 [SMILES: COc1ccc(Sc2sc(C(C)=O)cc2[N+](=O)[O-])cc1] | − |
| S18 | 4-(4-methoxyphenyl)- 2-methyl- 5-oxo-5H-indeno [1,2-b]pyridine- 3-carbonitrile | −112.41 | 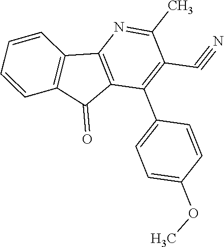 [SMILES: COc1ccc(-c2c(C#N)c(C)nc3c2C(=O)c2ccccc2-3)cc1] | − |
| S19 | 1-{5-[(2,3- dichlorophenyl) sulfanyl]-4- nitrothiophen-2- yl}ethan-1-one | −112.36 | 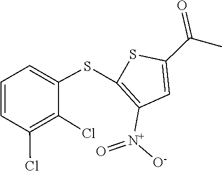 [SMILES: CC(=O)c1cc([N+](=O)[O-])c(Sc2cccc(Cl)c2Cl)s1] | − |
| S20 | 1- (1H- benzimidazol- 2-yl)ethanone (6-methyl-4- phenyl-2- quinazolinyl) hydrazone | −109.21 | 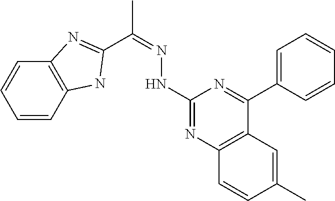 | − |
| S21 | 1-{5-[(4- chlorophenyl) sulfanyl]- 4-nitrothiophen- 2-yl}ethan-1- one | −109.19 | 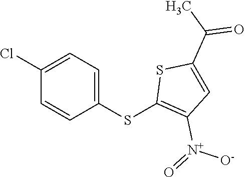 [SMILES: CC(=O)c1cc([N+](=O)[O-])c(Sc2ccc(Cl)cc2)s1] | − |
| S22 | Cryptochrysin | −108.43 | 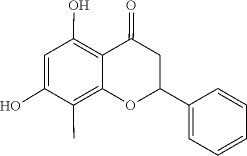 [SMILES: Cc1c(O)cc(O)c2c1OC(c1ccccc1)CC2=O] | − |
| S23 | 2-amino-4- (4-hydroxyphenyl) - 5-ox0-4H,5H- pyrano[3,2-c] chromene- 3-carbonitrile | −105.05 | 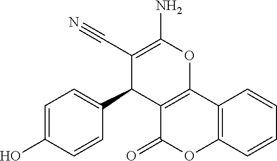 [SMILES: N#CC1=C(N)Oc2c(c(=O)oc3ccccc23)[C@H]1c1ccc(O)cc1] | − |
| S24 | alpha- naphthoflavanone | −104.67 | 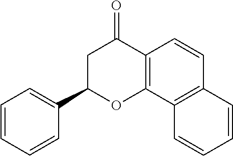 [SMILES: O=C1C[C@H](c2ccccc2)Oc2c1ccc1ccccc21] | − |
| S25 | ethyl 2-(4- ethoxyanilino)-5- [3-methoxy-4- (2-propynyloxy) benzylidene]-4- oxo-4,5- dihydro-3- thiophenecarboxylate | −103.73 | 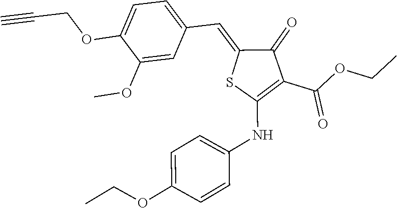 [SMILES: C#CCOc1ccc(/C=C2\SC(Nc3ccc(OCC)cc3)=C(C(=O)OCC)C2=O)cc1OC] | − |

TABLE 2
Antibodies

| Application | Target | Clone | Fluorophore | Vendor | Catalog No. |
|---|---|---|---|---|---|
| Flow Cytometry Antibodies | | | | | |
| Viability Dye | dead cells | N/A | Brilliant Violet 510 | Tonbo | 13-0870-T100 |
| Viability Dye | dead cells | N/A | Alexa Fluor 506 | eBioscience | 65-0866-14 |
| Mouse T Cell Surface Staining | CD90.1 (Tay1.1) | HIS51 | APC | eBioscience | 17-0900-82 |
| Mouse T Cell Surface Staining | CD3c | 145-2C11 | PE | Biolegend | 100307 |
| Mouse T Cell Surface Staining | CD3c | 17A2 | PE-Cy7 | Biolegend | 100219 |
| Mouse T Cell Surface Staining | CD3c | 145-2C11 | APC | Biolegend | 100311 |
| Mouse T Cell Surface Staining | CD4 | GK1.5 | Pacific Blue | BioLegend | 100428 |
| Mouse T Cell Surface Staining | CD4 | GK1.5 | PerCP-Cy5.5 | Biolegend | 100433 |
| Mouse T Cell Surface Staining | CD4 | GK1.5 | APC Cy7 | Biolegend | 100413 |
| Mouse T Cell Surface Staining | CD4 | GK1.5 | PE-Cy7 | Biolegend | 100421 |
| Mouse T Cell Surface Staining | CD8a | 53-6.7 | Pacific Blue | Biolegend | 100725 |
| Mouse T Cell Surface Staining | CD25 | PC61 | APC | BioLegend | 102012 |
| Mouse T Cell Surface Staining | CD25 | PC61 | PE-CY7 | Biolegend | 102016 |
| Mouse T Cell Surface Staining | CD25 | PC61 | PE | Biolegend | 102008 |
| Mouse T Cell Surface Staining | CD44 | IM7 | PE-Cy7 | Biolegend | 103030 |
| Mouse T Cell Surface Staining | CD44 | IM7 | APC | Biolegend | 103011 |
| Mouse T Cell Surface Staining | CD62L | MEL-14 | APC | BioLegend | 104412 |
| Mouse T Cell Surface Staining | CD62L | MEL-14 | PE | ebioscience | 12-0621-82 |
| Mouse T Cell Surface Staining | CD357 (GITR) | DTA-1 | PerCP-Cy5.5 | BioLegend | 126315 |
| Mouse T Cell Surface Staining | CD357 (GITR) | DTA-1 | PE | BioLegend | 126309 |
| Mouse T Cell Surface Staining | CD128 (ICOS) | 7E.17G9 | PE | ebioscience | 12-9942-81 |
| Mouse T Cell Surface Staining | CD279 (PD-1) | 29F.IA12 | PE | BioLegend | 135205 |
| Mouse T Cell Surface Staining | CD274 (PD-L1) | 10F.9G2 | PE | BioLegend | 124308 |
| Mouse T Cell Surface Staining | CD103 | 2E7 | PE | eBioscience | 12-1031-82 |
| Mouse T Cell Intracellular Staining | Foxp3 | FJK-16s | FITC | eBioscience | 11-5773-82 |
| Mouse T Cell Intracellular Staining | Foxp3 | FJK-16s | PE | eBioscience | 12-5773-82 |
| Mouse T Cell Intracellular Staining | Usp22 | C-3 | Alexa Fluor 647 | Santa Cruz Bio | sc-390585 |
| Mouse T Cell Intracellular Staining | IL-2 | JES6-5H4 | APC | eBioscience | 17-7021-81 |
| Mouse T Cell Intracellular Staining | IFN-γ | XMG1.2 | FITC | BioLegend | 505806 |
| Mouse T Cell Intracellular Staining | IL-4 | 11B11 | APC | BioLegend | 504105 |
| Mouse T Cell Intracellular Staining | IL-17A | TC11-18H10.1 | APC | BioLegend | 506915 |
| Mouse T Cell Intracellular Staining | Granzyme B | GB11 | FITC | BioLegend | 515403 |
| Mouse T Cell Intracellular Staining | Helios | 22F6 | APC | BioLegend | 137221 |
| Mouse T Cell Intracellular Staining | CD152 (CTLA-4) | UC10-4B9 | APC | BioLegend | 106309 |
| Mouse T Cell Intracellular Staining | CD152 (CTLA-4) | UC10-4B9 | PE-Cy7 | BioLegend | 106313 |
| Mouse T Cell Intracellular Staining | CD304 (Neuropilin-1) | 3E12 | APC | BioLegend | 145205 |
| Western, ChIP, and Co-IP Blot Antibodies | | | | | |
| HRP-conjugated Myc (Santa Cruz, Cat# 2040S) | | | | | |
| HRP-conjugated HA (Santa Cruz, Cat# 14031S) | | | | | |
| HRP-conjugatcd FLAG (Sigma, Cat# A8592) | | | | | |
| anti-GAPDH (Sigma, Cat# G9545) | | | | | |
| anti-Vinculin | | | | | |
| anti-Foxp3 (eBioscience, Cat# 11-5773-82) | | | | | |
| anti-Smad2 (Abcam, Cat# ab71109) | | | | | |
| anti-Smad3 (Abcam, Cat# ab28379) | | | | | |
| anti-Smad4 (Cell Signaling, Cat# 38454) | | | | | |
| anti-FLAG (Sigma, Cat# A8592) | | | | | |
| anti-Usp22 (Novus Biologicals, Cat# NBP1-49644) | | | | | |
| rabbit anti-IgG (Cell Signaling, Cat# 2729) | | | | | |
| rabbit anti-USP22 (Abcam, ab195289) | | | | | |

TABLE 3
Primers

| PRIMER NAME | SEQUENCE | USAGE | SEQ ID NO: |
|---|---|---|---|
| USP22Chp1F | TGTATTCTTGCCACGCCCAA | Smad ChIP | 3 |
| USP22Chp1R | TCCTAGTGTGGGCGTTTCTG | Smad ChIP | 4 |
| USP22Chp2F | ATTGCGGTACCCAACACAGT | Smad ChIP | 5 |
| USP22Chp2R | GCGTCTGCGAGTTCTCTGAA | Smad ChIP | 6 |
| USP22Chp3F | TTCAGAGAACTCGCAGACGC | Smad ChIP | 7 |
| USP22Chp3R | GCGTGCTGAGGATTGGGTAA | Smad ChIP | 8 |
| USP22Chp4F | TTACCCAATCCTCAGCACGC | Smad ChIP | 9 |
| USP22Chp4R | ATTGGTGGTTTGCCGGTCTA | Smad ChIP | 10 |
| USP22Chp5F | CTTAGACCGGCAAACCACCA | Smad ChIP | 11 |
| USP22Chp5R | GGCTCCAGAGAAAAGCCGAA | Smad ChIP | 12 |
| USP22Chp6F | TCTTAGACCGGCAAACCACC | Smad ChIP | 13 |
| USP22Chp6R | TGTCCGCGGGAAAGGATAAC | Smad ChIP | 14 |
| USP22Chp7F | TCCCACCTGTGTTGGATTGC | Smad ChIP | 15 |
| USP22Chp7R | GGGCTTCCCAAGACAATGACT | Smad ChIP | 16 |
| USP22Chp8F | AAGCCAAGGGCTTCCCAAG | Smad ChIP | 17 |
| USP22Chp8R | ACTCAGGGCATATTGTGAGGG | Smad ChIP | 18 |
| USP22Chp9F | TGTCGGCAATTTTTCTCGGC | Smad ChIP | 19 |
| USP22Chp9R | CCCATGATGTGGAGCAGTGA | Smad ChIP | 20 |
| USP21Pro1F | TGCATCGGCTAGGAATGGTC | Smad ChIP | 21 |
| USP21Pro1R | ACCAATCAGGTCACCAAGCC | Smad ChIP | 22 |
| USP21Pro2F | AGGCTTGGTGACCTGATTGG | Smad ChIP | 23 |
| USP21Pro2R | GCTTGTTCCGCAGATTCCAC | Smad ChIP | 24 |
| USP21Pro3F | AGCTCTCCTCTGTCAAGCCT | Smad ChIP | 25 |
| USP21Pro3R | AACGTAGAGCAGCCTCTTGG | Smad ChIP | 26 |
| USP21Pro4F | AGTGGAAGTCCCCGATCTGA | Smad ChIP | 27 |
| USP21Pro4R | GGCGTAGTCCTTCATTGGCT | Smad ChIP | 28 |
| USP21Pro5F | AGCCAATGAAGGACTACGCC | Smad ChIP | 29 |
| USP21Pro5R | CCTCCAGGGCTCTACTTGGA | Smad ChIP | 30 |
| USP21Pro6F | CCTGGTAGCCTGTGGTTCTC | Smad ChIP | 31 |
| USP21Pro6R | CTCCGCGTTTTGCTTGTTCA | Smad ChIP | 32 |
| USP21Pro7F | GGATCTCCCCACCCTTAGGT | Smad ChIP | 33 |
| USP21Pro7R | GGAAGCAAGAGGGATGCAGT | qPCR | 34 |
| Usp7F | AAGTCTCAAGGTTATAGGGA | qPCR | 35 |
| Usp7R | CCATGCTTGTCTGGGTATAGTGT | qPCR | 36 |
| USP21F | tgcatgaagaacctgagttga | qPCR | 37 |
| USP2IR | acaggtccacaatcttgctgt | qPCR | 38 |
| USP22ex2 1F | gcttcaaggtggacaactgg | qPCR | 39 |
| USP22ex2 1R | acatggcagacacaggactt | qPCR | 40 |
| hUSP22F 1 | GGAAAATGCAAGGCGTTGGAG | qPCR | 41 |
| hUSP22R_1 | GTGCAGTTGGAGGTGATCTTT | qPCR | 42 |
| hUSP22F 2 | CTGGGACATCAGCTTGGATCT | qPCR | 43 |
| hUSP22R_2 | CTTTCCCCGTTTACCACGTTG | qPCR | 44 |
| hUSP21F 1 | GCCACCCACTTTGAGACGTAG | qPCR | 45 |
| hUSP21R 1 | TCCGTATGCTGAACAGGGTAG | qPCR | 46 |
| hUSP7F | CCCTCCGTGTTTTGTGCGA | qPCR | 47 |
| hUSP7R | AGACCATGACGTGGAATCAGA | qPCR | 48 |
| h18S F | GAGGATGAGGTGGAACGTGT3 | qPCR | 49 |
| h18S R | AGAAGTGACGCAGCCCTCTA3 | qPCR | 50 |

1. A method of treating a subject in need of treatment for a disease or disorder associated with ubiquitin specific peptidase 22 (USP22) activity, the method comprising administering to the subject an effective amount of a therapeutic agent that inhibits the biological activity of USP22.

2. The method of claim 1, wherein the disease or disorder is a cell proliferative disease or disorder.

3. The method of claim 2, wherein the disease or disorder is cancer.

4. The method of claim 2, wherein the disease or disorder is a cancer selected from the group consisting of lung cancer, gastric carcinoma, pancreatic cancer, melanoma, lymphoma, colon cancer, breast cancer, ovarian cancer, bladder cancer, prostate cancer, glioma, mesothelioma, neuroblastoma, mantle cell lymphoma, and acute myeloid leukemia.

5. The method of claim 2, wherein the disease or disorder is lung cancer.

6. The method of claim 2, wherein the disease or disorder is melanoma.

7. The method of claim 1, wherein the therapeutic agent is a compound selected from the group consisting of:
7-(difluoromethyl)-N-(3,4-dimethylphenyl)-5-phenylpyrazolo[1,5-a]pyrimidine3-carboxamide,
11-Anilino-7,8,9,10-tetrahydrobenzimidazo[1,2-b]isoquinoline-6-carbonitrile,
2,7-bis(4-methoxyphenyl) 9-oxo9H-fluorene-2,7-disulfonate,
6-(2,5-dimethoxyphenyl)-2-oxo-1,2-dihydropyridine-3-carbonitrile,
2,4-dimethanesulfonyl-8-methoxy5H,6H-benzo[h]quinazoline,
4,5-bis(4-methoxyphenoxy)benzene-1,2-dicarbonitrile,
9-[(3-methylbut-2-en-1-yl)oxy]-7Hfuro[3,2-g]chromen-7-one,
N-(2-{[5-(ethanesulfonyl)-3-nitrothiophen-2-yl]sulfanyl}phenyl)acetamide,
1-[4-nitro-5-(pyridin-4-yl sulfanyl)thiophen-2-yl]ethan-1-one,
bis[(4-methoxyphenyl)amino]pyrazine2,3-dicarbonitrile,
5-{[(2,4-dimethylphenyl)sulfonyl]amino}-2-methyl-N-phenylnaphtho[1,2-b]furan-3-carboxamide,
8-Oxotetrahydropalmatine,
1-{5-[(4-chlorophenyl)amino]-4-nitrothiophen-2-yl}ethan-1-one,
Ethyl 6-cyano-7-(4-methoxyphenyl)-5-oxo-1-phenyl-1,5-dihydro[1,2,4]triazolo[4,3-a]pyrimidine-3-carboxylate,
1-(5-{[(4-chlorophenyl)methyl]sulfanyl}-4-nitrothiophen-2-yl)ethan-1-one,
bis[(3-chlorophenyl)amino]pyrazine-2,3-dicarbonitrile,
1-{5-[(4-methoxyphenyl)sulfanyl]-4-nitrothiophen-2-yl}ethan-1-one,
4-(4-methoxyphenyl)-2-methyl-5-oxo-5H-indeno[1,2-b]pyridine-3-carbonitrile,
1-{5-[(2,3-dichlorophenyl)sulfanyl]-4-nitrothiophen-2-yl}ethan-1-one,
1-(1H-benzimidazol-2-yl)ethanone (6-methyl-4-phenyl-2-quinazolinyl) hydrazone,
1-{5-[(4-chlorophenyl)sulfanyl]-4-nitrothiophen-2-yl}ethan-1-one,
Cryptochrysin,
2-amino-4-(4-hydroxyphenyl)-5-oxo-4H,5H-pyrano[3,2-c]chromene-3-carbonitrile,
alpha-naphthoflavanone, and
ethyl 2-(4-ethoxyanilino)-5-[3-methoxy-4-(2-propynyloxy) benzylidene]-4-oxo-4,5-dihydro-3-thiophenecarboxylate.

8. The method of claim 1, wherein the therapeutic agent is 11-Anilino-7,8,9,10-tetrahydrobenzimidazo[1,2-b]isoquinoline-6-carbonitrile.

9. The method of claim 1, wherein the therapeutic agent inhibits ubiquitin specific peptidase activity (E.C. 3.4.19.12) of USP22.

10. A method of suppressing Treg cell activity in a subject in need thereof, the method comprising administering to the subject an effective amount of a therapeutic agent that inhibits the activity of USP22.

11. The method of claim 10, wherein the subject has an infectious disease.

12. The method of claim 10, wherein the subject has sudden acute respiratory syndrome coronavirus 2 (SARS-CoV2) infection.

13. The method of claim 10, wherein the therapeutic agent is a compound selected from the group consisting of:
7-(difluoromethyl)-N-(3,4-dimethylphenyl)-5-phenylpyrazolo[1,5-a]pyrimidine3-carboxamide,
11-Anilino-7,8,9,10-tetrahydrobenzimidazo[1,2-b]isoquinoline-6-carbonitrile,
2,7-bis(4-methoxyphenyl) 9-oxo9H-fluorene-2,7-disulfonate,
6-(2,5-dimethoxyphenyl)-2-oxo-1,2-dihydropyridine-3-carbonitrile,
2,4-dimethanesulfonyl-8-methoxy5H,6H-benzo[h]quinazoline,
4,5-bis(4-methoxyphenoxy)benzene-1,2-dicarbonitrile,
9-[(3-methylbut-2-en-1-yl)oxy]-7Hfuro[3,2-g]chromen-7-one,
N-(2-{[5-(ethanesulfonyl)-3-nitrothiophen-2-yl]sulfanyl}phenyl)acetamide,
1-[4-nitro-5-(pyridin-4-yl sulfanyl)thiophen-2-yl]ethan-1-one,
bis[(4-methoxyphenyl)amino]pyrazine2,3-dicarbonitrile,
5-{[(2,4-dimethy]phenyl)sulfonyl}amino-2-methyl-N-phenylnaphtho[1,2-b]furan-3-carboxamide,
8-Oxotetrahydropalmatine,
1-{5-[(4-chlorophenyl)amino]-4-nitrothiophen-2-yl}ethan-1-one,
ethyl 6-cyano-7-(4-methoxyphenyl)-5-oxo-1-phenyl-1,5-dihydro[1,2,4]triazolo[4,3-a]pyrimidine-3-carboxylate,
1-(5-{[(4-chlorophenyl)methyl]sulfanyl}-4-nitrothiophen-2-yl)ethan-1-one,
bis[(3-chlorophenyl)amino]pyrazine-2,3-dicarbonitrile,
1-{5-[(4-methoxyphenyl)sulfanyl]-4-nitrothiophen-2-yl}ethan-1-one,
4-(4-methoxyphenyl)-2-methyl-5-oxo-5H-indeno[1,2-b]pyridine-3-carbonitrile,
1-{5-[(2,3-dichlorophenyl)sulfanyl]-4-nitrothiophen-2-yl}ethan-1-one,
1-(1H-benzimidazol-2-yl)ethanone (6-methyl-4-phenyl-2-quinazolinyl) hydrazone,
1-{5-[(4-chlorophenyl)sulfanyl]-4-nitrothiophen-2-yl}ethan-1-one,
Cryptochrysin,
2-amino-4-(4-hydroxyphenyl)-5-oxo-4H,5H-pyrano[3,2-c]chromene-3-carbonitrile,
alpha-naphthoflavanone, and
ethyl 2-(4-ethoxyanilino)-5-[3-methoxy-4-(2-propynyloxy) benzylidene]-4-oxo-4,5-dihydro-3-thiophenecarboxylate.

14. The method of claim 10, wherein the therapeutic agent is 11-Anilino-7,8,9,10-tetrahydrobenzimidazo[1,2-b]isoquinoline-6-carbonitrile.

15. The method of claim 10, wherein the therapeutic agent inhibits ubiquitin specific peptidase activity (E.C. 3.4.19.12) of USP22.

16. A method for inhibiting ubiquitin specific peptidase activity (E.C. 3.4.19.12) of USP22 in a subject in need thereof, the method comprising administering to the subject an effective amount of a therapeutic agent that inhibits the biological activity of USP22.

17. The method of claim 16, wherein the therapeutic agent is a compound selected from the group consisting of:
7-(difluoromethyl)-N-(3,4-dimethylphenyl)-5-phenylpyrazolo[1,5-a]pyrimidine3-carboxamide,
11-Anilino-7,8,9,10-tetrahydrobenzimidazo[1,2-b]isoquinoline-6-carbonitrile,
2,7-bis(4-methoxyphenyl) 9-oxo9H-fluorene-2,7-disulfonate,
6-(2,5-dimethoxyphenyl)-2-oxo-1,2-dihydropyridine-3-carbonitrile,
2,4-dimethanesulfonyl-8-methoxy5H,6H-benzo[h]quinazoline,
4,5-bis(4-methoxyphenoxy)benzene-1,2-dicarbonitrile,
9-[(3-methylbut-2-en-1-yl)oxy]-7Hfuro[3,2-g]chromen-7-one,
N-(2-{[5-(ethanesulfonyl)-3-nitrothiophen-2-yl]sulfanyl}phenyl)acetamide,
1-[4-nitro-5-(pyridin-4-yl sulfanyl)thiophen-2-yl]ethan-1-one,
bis[(4-methoxyphenyl)amino]pyrazine2,3-dicarbonitrile,
5-{[(2,4-dimethylphenyl)sulfonyl]amino}-2-methyl-N-phenylnaphtho[1,2-b]furan-3-carboxamide,
8-Oxotetrahydropalmatine,
1-{5-[(4-chlorophenyl)amino]-4-nitrothiophen-2-yl}ethan-1-one,
ethyl 6-cyano-7-(4-methoxyphenyl)-5-oxo-1-phenyl-1,5-dihydro[1,2,4]triazolo[4,3-a]pyrimidine-3-carboxylate,
1-(5-{[(4-chlorophenyl)methyl]sulfanyl}-4-nitrothiophen-2-yl)ethan-1-one,
bis[(3-chlorophenyl)amino]pyrazine-2,3-dicarbonitrile,
1-{5-[(4-methoxyphenyl)sulfanyl]-4-nitrothiophen-2-yl}ethan-1-one,
4-(4-methoxyphenyl)-2-methyl-5-oxo-5H-indeno[1,2-b]pyridine-3-carbonitrile,
1-{5-[(2,3-dichlorophenyl)sulfanyl]-4-nitrothiophen-2-yl}ethan-1-one,
1-(1H-benzimidazol-2-yl)ethanone (6-methyl-4-phenyl-2-quinazolinyl) hydrazone,
1-{5-[(4-chlorophenyl)sulfanyl]-4-nitrothiophen-2-yl}ethan-1-one,
Cryptochrysin,
2-amino-4-(4-hydroxyphenyl)-5-oxo-4H,5H-pyrano[3,2-c]chromene-3-carbonitrile,
alpha-naphthoflavanone, and
ethyl 2-(4-ethoxyanilino)-5-[3-methoxy-4-(2-propynyloxy) benzylidene]-4-oxo-4,5-dihydro-3-thiophenecarboxylate.

18. The method of claim 16, wherein the therapeutic agent is 11-Anilino-7,8,9,10-tetrahydrobenzimidazo[1,2-b]isoquinoline-6-carbonitrile.

19. A pharmaceutical composition comprising: (i) a therapeutic agent and (ii) a suitable pharmaceutical carrier, wherein the therapeutic agent is the compound according to claim 7.

20. The pharmaceutical composition of claim 19, wherein the compound is 11-Anilino-7,8,9,10-tetrahydrobenzimidazo[1,2-b]isoquinoline-6-carbonitrile.

21. The pharmaceutical composition of claim 19, wherein the composition comprises an effective amount of the compound for inhibiting biological activity of USP22 when administered to a subject in need thereof.

22. The pharmaceutical composition of claim 19, wherein the composition comprises an effective amount of the compound for suppressing Treg cell activity in a subject in need thereof.

23. The pharmaceutical composition of claim 19, wherein the composition comprises an effective amount of the compound for inhibiting ubiquitin specific peptidase activity (E.C. 3.4.19.12) of USP22 in a subject in need thereof.


SEQUENCE LISTING (50 sequences)

SEQ ID NO: 1 - protein, 525 aa, Homo sapiens
MVSRPEPEGEAMDAELAVAPPGCSHLGSFKVDNWKQNLRAIYQCFVWSGTAEARKRKAKSCICHVCGVHLNRLHSCLYCVFFGCFTKKHIHEHAKAKRHNLAIDLMYGGIYCFLCQDYIYDKDMEIIAKEEQRKAWKMQGVGEKFSTWEPTKRELELLKHNPKRRKITSNCTIGLRGLINLGNTCFMNCIVQALTHTPLLRDFFLSDRHRCEMQSPSSCLVCEMSSLFQEFYSGHRSPHIPYKLLHLVWTHARHLAGYEQQDAHEFLIAALDVLHRHCKGDDNGKKANNPNHCNCIIDQIFTGGLQSDVTCQVCHGVSTTIDPFWDISLDLPGSSTPFWPLSPGSEGNVVNGESHVSGTTTLTDCLRRFTRPEHLGSSAKIKCSGCHSYQESTKQLTMKKLPIVACFHLKRFEHSAKLRRKITTYVSFPLELDMTPFMASSKESRMNGQYQQPTDSLNNDNKYSLFAVVNHQGTLESGHYTSFIRQHKDQWFKCDDAIITKASIKDVLDSEGYLLFYHKQFLEYE

SEQ ID NO: 2 - protein, 513 aa, Homo sapiens
MAPGWPSLSAGSRQEAPQLAAGGSAYQAVGRQFQPRATALQGPSQAKSCICHVCGVHLNRLHSCLYCVFFGCFTKKHIHEHAKAKRHNLAIDLMYGGIYCFLCQDYIYDKDMEIIAKEEQRKAWKMQGVGEKFSTWEPTKRELELLKHNPKRRKITSNCTIGLRGLINLGNTCFMNCIVQALTHTPLLRDFFLSDRHRCEMQSPSSCLVCEMSSLFQEFYSGHRSPHIPYKLLHLVWTHARHLAGYEQQDAHEFLIAALDVLHRHCKGDDNGKKANNPNHCNCIIDQIFTGGLQSDVTCQVCHGVSTTIDPFWDISLDLPGSSTPFWPLSPGSEGNVVNGESHVSGTTTLTDCLRRFTRPEHLGSSAKIKCSGCHSYQESTKQLTMKKLPIVACFHLKRFEHSAKLRRKITTYVSFPLELDMTPFMASSKESRMNGQYQQPTDSLNNDNKYSLFAVVNHQGTLESGHYTSFIRQHKDQWFKCDDAIITKASIKDVLDSEGYLLFYHKQFLEYE

SEQ ID NO: 3 - DNA, 20 bp, Artificial Sequence
  Synthetic- USP22Chp1F
tgtattcttgccacgcccaa

SEQ ID NO: 4 - DNA, 20 bp, Artificial Sequence
  Synthetic- USP22Chp1R
tcctagtgtgggcgtttctg

SEQ ID NO: 5 - DNA, 20 bp, Artificial Sequence
  Synthetic- USP22Chp2F
attgcggtacccaacacagt

SEQ ID NO: 6 - DNA, 20 bp, Artificial Sequence
  Synthetic- USP22Chp2R
gcgtctgcgagttctctgaa

SEQ ID NO: 7 - DNA, 20 bp, Artificial Sequence
  Synthetic- USP22Chp3F
ttcagagaactcgcagacgc

SEQ ID NO: 8 - DNA, 20 bp, Artificial Sequence
  Synthetic- USP22Chp3R
gcgtgctgaggattgggtaa

SEQ ID NO: 9 - DNA, 20 bp, Artificial Sequence
  Synthetic- USP22Chp4F
ttacccaatcctcagcacgc

SEQ ID NO: 10 - DNA, 20 bp, Artificial Sequence
  Synthetic- USP22Chp4R
attggtggtttgccggtcta

SEQ ID NO: 11 - DNA, 20 bp, Artificial Sequence
  Synthetic- USP22Chp5F
cttagaccggcaaaccacca

SEQ ID NO: 12 - DNA, 20 bp, Artificial Sequence
  Synthetic- USP22Chp5R
ggctccagagaaaagccgaa

SEQ ID NO: 13 - DNA, 20 bp, Artificial Sequence
  Synthetic- USP22Chp6F
tcttagaccggcaaaccacc

SEQ ID NO: 14 - DNA, 20 bp, Artificial Sequence
  Synthetic- USP22Chp6R
tgtccgcgggaaaggataac

SEQ ID NO: 15 - DNA, 20 bp, Artificial Sequence
  Synthetic- USP22Chp7F
tcccacctgtgttggattgc

SEQ ID NO: 16 - DNA, 21 bp, Artificial Sequence
  Synthetic- USP22Chp7R
gggcttcccaagacaatgact

SEQ ID NO: 17 - DNA, 19 bp, Artificial Sequence
  Synthetic- USP22Chp8F
aagccaagggcttcccaag

SEQ ID NO: 18 - DNA, 21 bp, Artificial Sequence
  Synthetic- USP22Chp8R
actcagggcatattgtgaggg

SEQ ID NO: 19 - DNA, 20 bp, Artificial Sequence
  Synthetic- USP22Chp9F
tgtcggcaatttttctcggc

SEQ ID NO: 20 - DNA, 20 bp, Artificial Sequence
  Synthetic- USP22Chp9R
cccatgatgtggagcagtga

SEQ ID NO: 21 - DNA, 20 bp, Artificial Sequence
  Synthetic- USP21Pro1F
tgcatcggctaggaatggtc

SEQ ID NO: 22 - DNA, 20 bp, Artificial Sequence
  Synthetic- USP21Pro1R
accaatcaggtcaccaagcc

SEQ ID NO: 23 - DNA, 20 bp, Artificial Sequence
  Synthetic- USP21Pro2F
aggcttggtgacctgattgg

SEQ ID NO: 24 - DNA, 20 bp, Artificial Sequence
  Synthetic- USP21Pro2R
gcttgttccgcagattccac

SEQ ID NO: 25 - DNA, 20 bp, Artificial Sequence
  Synthetic- USP21Pro3F
agctctcctctgtcaagcct

SEQ ID NO: 26 - DNA, 20 bp, Artificial Sequence
  Synthetic- USP21Pro3R
aacgtagagcagcctcttgg

SEQ ID NO: 27 - DNA, 20 bp, Artificial Sequence
  Synthetic- USP21Pro4F
agtggaagtccccgatctga

SEQ ID NO: 28 - DNA, 20 bp, Artificial Sequence
  Synthetic- USP21Pro4R
ggcgtagtccttcattggct

SEQ ID NO: 29 - DNA, 20 bp, Artificial Sequence
  Synthetic- USP21Pro5F
agccaatgaaggactacgcc

SEQ ID NO: 30 - DNA, 20 bp, Artificial Sequence
  Synthetic- USP21Pro5R
cctccagggctctacttgga

SEQ ID NO: 31 - DNA, 20 bp, Artificial Sequence
  Synthetic- USP21Pro6F
cctggtagcctgtggttctc

SEQ ID NO: 32 - DNA, 20 bp, Artificial Sequence
  Synthetic- USP21Pro6R
ctccgcgttttgcttgttca

SEQ ID NO: 33 - DNA, 20 bp, Artificial Sequence
  Synthetic- USP21Pro7F
ggatctccccacccttaggt

SEQ ID NO: 34 - DNA, 20 bp, Artificial Sequence
  Synthetic- USP21Pro7R
ggaagcaagagggatgcagt

SEQ ID NO: 35 - DNA, 20 bp, Artificial Sequence
  Synthetic- Usp7F
aagtctcaaggttataggga

SEQ ID NO: 36 - DNA, 23 bp, Artificial Sequence
  Synthetic- Usp7R
ccatgcttgtctgggtatagtgt

SEQ ID NO: 37 - DNA, 21 bp, Artificial Sequence
  Synthetic- USP21F
tgcatgaagaacctgagttga

SEQ ID NO: 38 - DNA, 21 bp, Artificial Sequence
  Synthetic- USP21R
acaggtccacaatcttgctgt

SEQ ID NO: 39 - DNA, 20 bp, Artificial Sequence
  Synthetic- USP22ex2 1F
gcttcaaggtggacaactgg

SEQ ID NO: 40 - DNA, 20 bp, Artificial Sequence
  Synthetic- USP22ex2 1R
acatggcagacacaggactt

SEQ ID NO: 41 - DNA, 21 bp, Artificial Sequence
  Synthetic- hUSP22F 1
ggaaaatgcaaggcgttggag

SEQ ID NO: 42 - DNA, 21 bp, Artificial Sequence
  Synthetic- hUSP22R_ 1
gtgcagttggaggtgatcttt

SEQ ID NO: 43 - DNA, 21 bp, Artificial Sequence
  Synthetic- hUSP22F 2
ctttccccgtttaccacgttg

SEQ ID NO: 44 - DNA, 21 bp, Artificial Sequence
  Synthetic- hUSP22R_ 2
ctttccccgtttaccacgttg

SEQ ID NO: 45 - DNA, 21 bp, Artificial Sequence
  Synthetic- hUSP21F 1
gccacccactttgagacgtag

SEQ ID NO: 46 - DNA, 21 bp, Artificial Sequence
  Synthetic- hUSP21R 1
tccgtatgctgaacagggtag

SEQ ID NO: 47 - DNA, 19 bp, Artificial Sequence
  Synthetic- hUSP7F
ccctccgtgttttgtgcga

SEQ ID NO: 48 - DNA, 21 bp, Artificial Sequence
  Synthetic- hUSP7R
agaccatgacgtggaatcaga

SEQ ID NO: 49 - DNA, 20 bp, Artificial Sequence
  Synthetic- h18S F
gaggatgaggtggaacgtgt

SEQ ID NO: 50 - DNA, 20 bp, Artificial Sequence
  Synthetic- h18S R
agaagtgacgcagccctcta